(19) United States
(12) Patent Application Publication
(10) Pub. No.: US 2010/0125073 A1
(43) Pub. Date: May 20, 2010

(54) BENZOFURAN AND BENZOTHIOPHENE DERIVATIVES USEFUL IN THE TREATMENT OF CANCERS OF THE CENTRAL NERVOUS SYSTEM

(76) Inventors: Olaf Weber, Wulfrath (DE); Verena Voehringer, Wuppertal (DE); Hans-Georg Lerchen, Leverkusen (DE); Frank-Thorsten Hafner, Wuppertal (DE); Joerg Keldenich, Wuppertal (DE); Karl-Heinz Schlemmer, Wuppertal (DE); Ursula Krenz, Leichlingen (DE); Bernd Riedl, Wuppertal (DE)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/439,062

(22) PCT Filed: Aug. 28, 2007
(86) PCT No.: PCT/EP07/07499

§ 371 Date: Jan. 14, 2010

(30) Foreign Application Priority Data
Sep. 1, 2006 (EP) EP 06018281.3

(51) Int. Cl. A61K 31/5377; C07D 307/82; A61K 31/343; C07D 413/10; A61K 31/422; A61K 31/427; C07D 417/10; C07D 405/10; A61K 31/443; C07D 413/12; A61P 35/00

(52) U.S. Cl. 514/233.5; 549/467; 514/470; 548/236; 514/374; 514/365; 548/204; 546/284.1; 514/337; 544/153

(57) ABSTRACT

The present invention relates to benzofuran and benzothiophene derivatives and compositions containing such compounds for the production of medicaments for the treatment of cancers of the central nervous system as monotherapy or combination with other agents.

The present invention relates to benzofuran and benzothiophene derivatives and compositions containing such compounds for the production of medicaments for the treatment of cancers of the central nervous system as monotherapy or combination with other agents.

WO 03/072561 describes benzofuran and benzothiophene compounds, pharmaceutical compositions containing such compounds, the process for preparing those compounds and the use of those compounds and/or compositions for treating hyper-proliferative disorders.

Gliomas are tumors of the brain that come from glia and, therefore, are of neuroepithelial origin. These tumors comprise 30-50% of all diagnosed tumors of the brain. Gliomas are classified according to a WHO system (Kleihues P, Burger P C, Scheithauer B W (1993) The New WHO Classification of Brain Tumours. Brain Pathology 3: 255-268). According to the classification there exist four different grades of malignity: Approximately 50% are highly malign glioblastomas (Grade IV), approx. 25% are astrocytomas grade I to III, 5-18% are oligodendrogiomas and 2-9% are so-called Ependymomas. The incidence in EU and U.S. are approximately 7-11/100,000 people, men are more often afflicted than women.

Current therapies focus on surgical resection of the tumor tissues and subsequent radiological therapy. This approach may work for grade I/II stages but has certain limitations if the tumor has spread. Chemotherapy is indicated for malign glioblastomas. The treatment might be complicated by the fact that a number of therapeutic agents do not cross the blood brain barrier. In addition to chemotherapy, symptomatic treatment (anti-epileptic treatments, treatment of edemas, antithrombotic treatments) might be required. Grade II to IV disease is complicated by the infiltration of the tumor into the brain tissues and these patients may receive only palliative treatments.

The invention relates to substituted benzofuran and benzothiophene derivatives that have utility in the treatment of cancers of the central nervous system, said derivatives having Formula I

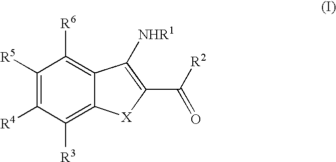

wherein
X is selected from O and S;
R¹ is selected from H, (C₁-C₆)alkyl, C(O)(C₁-C₆)alkyl, and benzoyl;
R² is selected from
   phenyl and naphthyl, each optionally substituted with 1, 2, or 3 substituents each independently selected from OH, CN, NO₂, (C₁-C₆)alkyl, (C₁-C₆)alkoxy, halo, halo(C₁-C₆)alkyl, halo(C₁-C₆)alkoxy, C(O)R^{A}, C(O)NR^{B}R^{B}, NR^{B}R^{B}, NH[(C₁-C₆)alkyl]₀₋₁S(O)₂R^{B}, NH[(C₁-C₆)alkyl]₀₋₁C(O)R^{A}, and NH[(C₁-C₆)alkyl]₀₋₁C(O)OR^{B},
   a heterocycle selected from a six membered heterocycle, a five membered heterocycle and a fused bicyclic heterocycle, each heterocycle being optionally substituted with 1, 2 or 3 substituents each independently selected from OH, CN, NO₂, (C₁-C₆)alkyl, (C₁-C₆)alkoxy, halo, halo(C₁-C₆)alkyl, halo(C₁-C₆)alkoxy, C(O)R^{A}, C(O)NR^{B}R^{B}, NR^{B}R^{B}, NH[(C₁-C₆)allkyl,]₀₋₁S(O)₂R^{B}, NH[(C₁-C₆)alkyl]₀₋₁C(O)R^{A}, and NH[(C₁-C₆)alkyl]₀₋₁C(O)OR^{B},
R^{A} is in each instance independently H, (C₁-C₆)alkyl, (C₁-C₆)alkoxy, NR^{B}R^{B}, or (C₁-C₆)alkyl, said alkyl being optionally substituted with OH, C(O)R^{B}, halo, (C₁-C₃)alkoxy, and NR^{B}R^{B};
R^{B} is in each instance independently H, (C₃-C₆)cycloalkyl, and (C₁-C₆)alkyl, said alkyl being optionally substituted with OH, ═O, halo, (C₁-C₆)alkoxy, NH(C₁-C₃)alkyl, N[(C₁-C₃)alkyl]₂, and NC(O)(C₁-C₃)alkyl,
   and where R^{B}, when it is attached to a N atom, is in each instance (C₁-C₄)alkyl, then the 2 (C₁-C₄)alkyl groups, taken together with the N atom to which they are attached, may be joined together to form a saturated ring,
   and where R^{B} and R^{B} together with the N to which they are attached may form a morpholinyl ring or a piperazinyl ring optionally substituted on the available N atom with (C₁-C₆)alkyl, said alkyl being optionally substituted with OH, ═O, NH₂, (C₁-C₆)alkoxy, NH(C₁-C₃)alkyl, or N[(C₁-C₃)alkyl]₂,
   and with the proviso that when R^{B} is attached to S(O) or to S(O)₂, it cannot be H;
R³ is selected from H, OH, CN, (C₁-C₃)alkyl, (C₁-C₃)alkoxy, halo, halo(C₁-C₃)alkyl, and halo(C₁-C₃)alkoxy;
R⁴ is selected from
   piperonyl,
   Y where
      Y is a heterocycle optionally substituted with 1, 2, or 3 substituents each independently selected from ═O, N-oxide, H, CN, NO₂, halo, halo(C₁-C₆)alkyl, OH, halo(C₁-C₆)alkoxy, C(O)OR^{B}, C(NH)NR^{B}R^{B}, NR^{B}R^{B}, S(O)₀₋₂R^{B}, S(O)₂NR^{B}R^{B}, (C₁-C₆)alkoxy, NR^{C}R^{C}, NR^{B}R^{E}, (C₁-C₆)alkyl, C(O)R^{D}
         where said alkoxy being optionally substituted with 1 or 2 substituents selected from OH, NR^{B}R^{B}, and (C₁-C₃)alkoxy,
         said alkyl being optionally substituted with CN, OH, ═O, halo, (C₁-C₆)alkoxy, C(O)R^{A}, NR^{B}R^{B}, NR^{C}R^{C}, NR^{B}R^{E}, C(NH)NR^{B}R^{B}, S(O)₀₋₂R^{B}, S(O)₂NR^{B}R^{B}, C(O)R^{B}C(O)OR^{B}, Z, C(O)Z, and C(O)N[(C₁-C₃)alkyl]Z, where Z in each instance is independently optionally substituted as described above,
         R^{C} is selected from R^{B}, C(O)R^{B}, and S(O)₂R^{B},
         R^{D} is selected from R^{A}, (C₃-C₆)cycloalkyl, Z and N[(C₁-C₃)alkyl]Z where
            Z is in each instance a heterocycle independently optionally substituted with CN, ═O, OH, N-oxide, NO₂, halo, (C₁-C₆)alkoxy, halo(C₁-C₃)alkoxy, halo(C₁-C₃)alkyl, S(O)₂R^{B}, S(O)₂NR^{B}R^{B}, NR^{B}R^{B}, C(O)R^{A}, and (C₁-C₆)alkyl, said alkyl being optionally substituted with OH, C(O)R^{B}, (C₁-C₃)alkoxy and NR^{B}R^{B};
         R^{E} is selected from C(O)R^{A}, C(O)R^{B}, S(O)₂R^{B}, S(O)₂NR^{B}R^{B} and C(O)[(C₁-C₆)alkyl]Z where Z is optionally substituted as described above,
   phenyl and naphthyl each optionally substituted with 1, 2, or 3 substituents each independently selected from OH, CN, NO₂, halo, halo(C₁-C₆)alkyl, halo(C₁-C₆)alkoxy, C(O)OR^{B}, NR^{C}R^{C}, NR^{B}R^{E}, C(O)R^{D}, (C₁-C₆)alkoxy, C(NH)NR^{B}R^{B}, NR^{B}R^{B}, S(O)₀₋₂R^{B}, S(O)₂NR^{B}R^{B}, (C₁-C₆)alkyl, Z, C(O)Z
      where Z is in each instance optionally substituted as described above,
      said alkoxy being optionally substituted with 1 or 2 substituents selected from OH, NR^{B}R^{B}, and (C₁-C₃)alkoxy,
      R^{C} is selected from R^{B}, C(O)R^{B}, and S(O)₂R^{B},
      R^{D} is selected from R^{A}, (C₃-C₆)cycloalkyl, and N[(C₁-C₃)alkyl]Z where Z is optionally substituted as described above,
      R^{E} is selected from C(O)R^{A}, C(O)R^{B}, S(O)₂R^{B}, S(O)₂NR^{B}R^{B} and C(O)[(C₁-C₆)alkyl]Z where Z is optionally substituted as described above,
      said alkyl being optionally substituted with CN, OH, ═O, halo, (C₁-C₆)alkoxy, C(O)R^{A}, NR^{B}R^{B}, NR^{B}R^{E}, C(NH)NR^{B}R^{B}, S(O)₀₋₂R^{D}, S(O)₂NR^{B}R^{B}, C(O)R^{B}C(O)OR^{B}, Z, C(O)Z, and C(O)N[(C₁-C₃)alkyl]Z, where Z in each instance is independently optionally substituted as described above;
R⁵ and R⁶ are each independently selected from H, OH, CN, (C₁-C₃)alkyl, (C₁-C₃)alkoxy, halo, halo(C₁-C₃)alkyl, and halo(C₁-C₃)alkoxy;
   or a pharmaceutically acceptable salt, prodrug or ester thereof.

Most preferred as compound of formula (I) is N-{3-[3-amino-2-(2,4-dichlorobenzoyl)-1-benzofuran-6-yl]benzyl}methanesulfonamide or a pharmaceutically acceptable salt, prodrug or ester thereof.

Another embodiment of the present invention is a compound of the formula (IX)

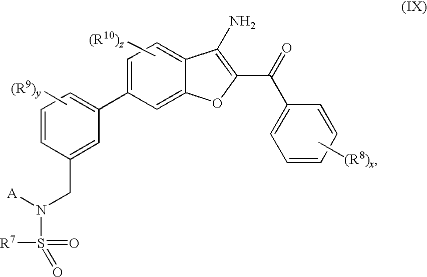

wherein
A is an alpha amino acid residue, which is linked via the α-carboxyl function, and which can optionally carry one or more protective groups,
R⁷ is H or (C₁-C₆)alkyl,
R⁸, R⁹ and R¹⁰ are independently selected from CN, NO₂, (C₁-C₆)alkyl, (C₁-C₆)alkoxy, halo, halo(C₁-C₆)alkyl and halo(C₁-C₆)alkoxy, and
x, y, z are independently 1, 2, or 3,
or a pharmaceutical acceptable salt thereof.

Preference is given to compounds of the formula (IX) wherein
A is a naturally occurring alpha amino acid residue of the D or L configuration, which is linked via the α-carboxyl function, and which can optionally carry one or more protective groups,
and the other radicals are as defined above.

Particular preference is given to compounds of the formula (IX) wherein
A is a naturally occurring alpha amino acid residue of the D or L configuration selected from the amino acids glycine, alanine, leucine, valine, norleucine, isoleucine, D-allo isoleucine, lysine, histidine, ornithine, arginine, aspartic acid, asparagine, glutamic acid, glutamine, serine, threonine, phenylalanine and tyrosine, which is linked via the α-carboxyl function, and which can optionally carry one or more protective groups,
   and the other radicals are as defined above.
   Most preferred as compounds of formula (IX) are
N-{3-[3-amino-2-(2,4-dichlorobenzoyl)-1-benzofuran-6-yl]benzyl}-N-(methylsulfonyl)-L-valinamide,
N-{3-[3-amino-2-(2,4-dichlorobenzoyl)-1-benzofuran-6-yl]benzyl}-N-(methylsulfonyl)-L-isoleucinamide,
N-{3-[3-amino-2-(2,4-dichlorobenzoyl)-1-benzofuran-6-yl]benzyl}-N-(methylsulfonyl)-D-alloisoleucinamide,
N-{3-[3-amino-2-(2,4-dichlorobenzoyl)-1-benzofuran-6-yl]benzyl}-O-benzyl-N-(methylsulfonyl)-L-serinamide,
Benzyl N-{3-[3-amino-2-(2,4-dichlorobenzoyl)-1-benzofuran-6-yl]benzyl}-N-(methylsulfonyl)-L-alpha-asparaginate,
N-{3-[3-amino-2-(2,4-dichlorobenzoyl)-1-benzofuran-6-yl]benzyl}-N-(methylsulfonyl)-D-valinamide,
N-{3-[3-amino-2-(2,4-dichlorobenzoyl)-1-benzofuran-6-yl]benzyl}-N-(methylsulfonyl)glycin amide,
N-{3-[3-amino-2-(2,4-dichlorobenzoyl)-1-benzofuran-6-yl]benzyl}-N-(methylsulfonyl)-L-lysinamide,
N-{3-[3-amino-2-(2,4-dichlorobenzoyl)-1-benzofuran-6-yl]benzyl}-N-(methylsulfonyl)-D-lysinamide,
N-{3-[3-amino-2-(2,4-dichlorobenzoyl)-1-benzofuran-6-yl]benzyl}-N-(methylsulfonyl)-D-leucinamide,
N-{3-[3-amino-2-(2,4-dichlorobenzoyl)-1-benzofuran-6-yl]benzyl}-N-(methylsulfonyl)-L-leucinamide,
N-{3-[3-amino-2-(2,4-dichlorobenzoyl)-1-benzofuran-6-yl]benzyl}-N-(methylsulfonyl)-L-alpha-asparagine,
N-{3-[3-amino-2-(2,4-dichlorobenzoyl)-1-benzofuran-6-yl]benzyl}-N-(methylsulfonyl)-D-alpha-asparagine,
   or a pharmaceutical acceptable salt thereof.

The compounds of formula (IX) are useful as prodrugs for the compounds of formula (I). Prodrugs are drug precursors which, following administration to a subject and subsequent absorption, is converted to an active species in vivo via some process, such as metabolic process. Other products from the conversion process are easily disposed of by the body. Prodrugs produce products from the conversion process which are generally accepted as safe.

The active species converted from a compound of formula (IX) is a compound of formula (X)

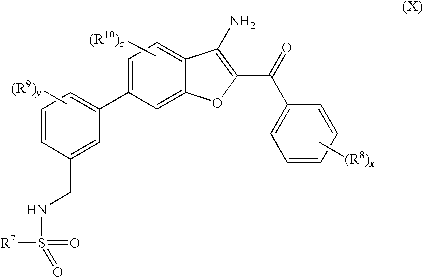

wherein R⁷, R⁸, R⁹, R¹⁰, x, y and z are defined as mentioned above.

Compounds of formula (X) and methods for their preparation are disclosed in WO 03/072561.

The terms identified above have the following meaning throughout:

The term “optionally substituted” means that the moiety so modified may have from none to up to about the highest number of substituents indicated. When there are two or more substituents on any moiety, each substituent is defined independently of any other substituent and can, accordingly, be the same or different.

The term “(C₁-C₆)alkyl, said alkyl being optionally substituted” means an alkyl group as defined below wherein each C atom is bonded to 0, 1, 2 or 3H atoms, as appropriate, and any up to all H atoms may be replaced with a recited substituent, with the proviso that combinations of recited substituents result in a chemically stable compound.

The terms “(C₁-C₆)alkyl”, “(C₁-C₄)alkyl”, and “(C₁-C₃)alkyl” mean linear or branched saturated carbon groups having from about 1 to about 3, 4, or 6 C atoms respectively. Such groups include but are not limited to methyl, ethyl, n-propyl, isopropyl, n-butyl, isobutyl, sec-butyl, tert-butyl, and the like.

The terms “(C₁-C₆)alkoxy” and “(C₁-C₃)alkoxy” mean a linear or branched saturated carbon group having from about 1 to about 6 or 3 C atoms, respectively, said carbon group being attached to an O atom. The O atom is the point of attachment of the alkoxy substituent. Such groups include but are not limited to methoxy, ethoxy, n-propoxy, isopropoxy, n-butoxy, isobutoxy, sec-butoxy, tert-butoxy, and the like.

The term “C₃-C₆ cycloalkyl” means a saturated monocyclic alkyl group of from 3 to about 8 carbon atoms and includes such groups as cyclopropyl, cyclopentyl, cyclohexyl, and the like.

The term “halo” means an atom selected from Cl, Br, F and I, where Cl, Br and F are preferred and Cl and F are most preferred.

The terms “halo(C₁-C₆)alkyl” and “halo(C₁-C₃)alkyl” mean a linear or branched saturated carbon group having from about 1 to about 6 or 3 C atoms, respectively, that is substituted with at least 1 and up to perhalo (that is, up to 3 per C atom, as appropriate) Cl or F atoms selected in each instance independently from any other Cl or F atom. Such groups include but are not limited to trifluoromethyl, trichloromethyl, pentafluoroethyl, fluorobutyl, 6-chlorohexyl, and the like.

The terms “halo(C₁-C₆)alkoxy” and “halo(C₁-C₃)alkoxy” mean a linear or branched saturated carbon group having from about 1 to about 6 or 3 C atoms, respectively, said carbon group being attached to an O atom and being substituted with at least 1 and up to perhalo (that is, up to 3 per C atom, as appropriate) Cl or F atoms selected in each instance independently from any other Cl or F atom. Such groups include but are not limited to trifluoromethoxy, trichloromethoxy, pentafluoroethoxy, fluorobutoxy, 6-chlorohexoxy, and the like.

The term “six membered heterocycle” means an aromatic ring made of 6 atoms, 1, 2, or 3 of which are N atoms, the rest being C, where the heterocycle is attached to the core molecule at any available C atom and is optionally substituted at any available C atom with the recited substituents. Such groups include pyridine, pyrimidine, pyridazine and triazine in all their possible isomeric forms.

The term “five membered heterocycle” means an aromatic ring made of 5 atoms and having 1, 2 or 3 heteroatom(s) each selected independently from O, N, and S, the rest being C atoms, with the proviso that there can be no more than 2 O atoms in the heterocycle and when there are 2 O atoms they must be nonadjacent. This heterocycle is attached to the core molecule at any available C atom and is optionally substituted at any available C or N atom with the recited substituents. Such groups include pyrrole, furan, thiophene, imidazole, pyrazole, thiazole, oxazole, isoxazole, isothiazole, triazole, oxadiazole, thiadiazole, and tetrazole in all their possible isomeric forms.

The term “fused bicyclic heterocycle” means a group having from 9 to 12 atoms divided into 2 rings that are fused together through adjacent C atoms where 1, 2, or 3 of the remaining atoms are heteroatoms each independently selected from N, O, and S. The heteroatoms may be located at any available position on the fused bicyclic moiety with the proviso that there can be no more than 2 O atoms in any fused bicyclic heterocycle, and when 2 O atoms are present, they must not be adjacent. At least one of the two fused rings must be aromatic. The other ring, if it were not fused to the aromatic ring, may be aromatic, partially saturated or saturated. An aromatic ring is always attached to the core molecule through any available C atom. The fused bicyclic heterocycle is optionally substituted at any available C atoms with the recited substituents. Such groups include 5-5, 5-6, and 6-6 fused bicycles, where one of the rings is one of the heterocycles described above and the second ring is either benzene or another heterocycle including, but not limited to, chroman, chromene, benzofuran, benzthiophene, quinoline, isoquinoline, phthalazine, naphthyridine, quinoxaline, purine, indole, indazole, isoindole, indolizine, cinnoline, pteridine, isoindole, thienofuran, imidazothiazole, dithianaphthalene, benzoxazine, piperonyl, and the like.

The term “Y is a heterocycle” means a saturated, partially unsaturated or aromatic ring containing about 5 or 6 atoms, 1, 2, or 3 of which are each independently selected from N, O, and S, the rest being C atoms, with the proviso that there can be no more than 2 O atoms in any heterocycle. When there are 2 O atoms in the heterocycle, they must be nonadjacent. This heterocycle is attached to the core molecule through any available C atom or, except where the heterocycle is pyridyl, through any available N atom. It is optionally substituted with the recited substituents on any available C atom and, except when the heterocycle is pyridyl, on any available N atom. This heterocycle includes furan, pyrrole, imidazole, pyrazole, isoxazole, pyridine, pyrazine, pyrimidine, pyridazine, furazan, pyrrolidine, imidazolidine, imidazoline, pyrazoline, piperidine, morpholine, oxathiazine, oxazine, triazine, piperizine, dioxazole, oxathiole, pyran, dithiole, and the like.

The term “Z is a heterocycle” means a saturated, partially unsaturated or aromatic ring containing about 5 or 6 atoms, 1, 2, or 3 of which are each independently selected from N, O, and S, the rest being C atoms, with the proviso that there can be no more than 2 O atoms in any heterocycle. When there are 2 O atoms in the heterocycle, they must be nonadjacent. This heterocycle is attached to the core molecule through any available C atom or, except where the heterocycle is pyridyl, through any available N atom. It is optionally substituted with the recited substituents on any available C atom and, except when the heterocycle is pyridyl, on any available N atom. This heterocycle includes furan, pyrrole, imidazole, pyrazole, isoxazole, pyridine, pyrazine, pyrimidine, pyridazine, furazan, pyrrolidine, imidazolidine, imidazoline, pyrazoline, piperidine, morpholine, oxathiazine, oxazine, triazine, piperizine, dioxazole, oxathiole, pyran, dithiole, and the like.

The term “N-oxide” means that for heterocycles containing an otherwise unsubstituted sp² N atom, the N atom may bear a covalently bound O atom, i.e.,

—N(→O). Examples of such N-oxide substituted heterocycles include pyridyl N-oxides, pyrimidinyl N-oxides, pyrazinyl N-oxides and pyrazolyl N-oxides.

The term “piperonyl” means a methylenedioxyphenyl ring of structure

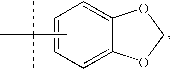

the point of attachment of which is any available aromatic C atom.

The term “alpha amino acid” refers according to the invention in particular to the alpha amino acids occurring in nature, but moreover also includes their homologues and isomers. The alpha amino acid can occur in the L or in the D configuration or alternatively as a mixture of the D and L form. The alpha amino acid residue is linked via its α-carboxyl function to the rest of the molecule.

Alpha amino acids according to the invention include but are not limited to alanine, valine, phenylalanine, tyrosine, threonine, serine, isoleucine, D-allo isoleucine, lysine, glutamic acid, histidine, glycine, arginine, asparagine, glutamine, S-methyl-cysteine, methionine, aspartic acid, tryptophane, proline, ornithine, norvaline leucine and norleucine. Preference is given to glycine, alanine, leucine, valine, norvaline, norleucine, isoleucine, D-allo isoleucine, lysine, histidine, ornithine, arginine, aspartic acid, asparagine, glutamic acid, glutamine, serine, threonine, phenylalanine and tyrosine.

In the case of amino acids having functional groups in the side chains, these functional groups can be either deblocked or protected by conventional protective groups known in peptide chemistry which are described in T. W. Greene and P. G. M. Wuts, Protective Groups in Organic Synthesis; Wiley: New York, (1999), and which are for example of the urethane, alkyl, acyl, ester or amide type.

Protective groups include but are not limited to for example benzyloxycarbonyl (Z), 3,4-dimethoxybenzyloxycarbonyl, tert-butoxycarbonyl (Boc), 3,5-dimethoxybenzyloxycarbonyl, 2,4-dimethoxybenzyloxycarbonyl, 4-methoxybenzyloxycarbonyl, 4-nitrobenzyloxycarbonyl, 2-nitrobenzyloxycarbonyl, 2-nitro-4,5-dimethoxybenzyloxycarbonyl, methoxycarbonyl, ethoxy-carbonyl, allyloxycarbonyl, vinyloxycarbonyl, 3,4,5-trimethoxybenzyloxycarbonyl, phthaloyl, 2,2,2-trichloroethoxycarbonyl, 2,2,2-trichloro-tert-butoxycarbonyl, menthyloxycarbonyl, 4-nitro-phenoxycarbonyl, fluorenyl-9-methoxycarbonyl (Fmoc), formyl, acetyl, propionyl, pivaloyl, 2-chloroacetyl, 2-bromoacetyl, 2,2,2-trifluoroacetyl, 2,2,2-trichloroacetyl, benzoyl, benzyl, 4-chlorobenzoyl, 4-bromobenzoyl, 4-nitrobenzoyl, phthalimido, isovaleroyl or benzyloxy-methylene, 4-nitrobenzyl, 2,4-dinitrobenzyl, 4-nitrophenyl or 2-nitrophenylsulphenyl. Preference is given to benzyloxycarbonyl (Z), tert-butoxycarbonyl (Boc), fluorenyl-9-methoxycarbonyl (Fmoc), formyl, benzoyl and benzyl.

The removal of protective groups in appropriate reaction steps can be carried out, for example, by the action of acid or base, hydrogenolytically or reductively in another manner as described in T. W. Greene and P. G. M. Wuts, Protective Groups in Organic Synthesis; Wiley: New York, (1999).

The use of pharmaceutically acceptable salts of the compounds of this invention are also within the scope of this invention. The term “pharmaceutically acceptable salt” refers to either inorganic or organic acid or base salts of a compound of the present invention that have properties acceptable for the therapeutic use intended. For example, see S. M. Berge, et al. “Pharmaceutical Salts,” J. Pharm. Sci. 1977, 66, 1-19.

The compounds of this invention may contain one or more asymmetric centers, depending upon the location and nature of the various substituents desired. Asymmetric carbon atoms may be present in the (R) or (S) configuration or (R,S) configuration. In certain instances, asymmetry may also be present due to restricted rotation about a given bond, for example, the central bond adjoining two substituted aromatic rings of the specified compounds. Substituents on a ring may also be present in either cis or trans form, and a substituent on a double bond may be present in either Z or E form. It is intended that all such configurations (including enantiomers and diastereomers) are included within the scope of the present invention. Preferred compounds are those with the absolute configuration of the compound of this invention which produces the more desirable biological activity. Separated, pure or partially purified isomers or racemic mixtures of the compounds of this invention are also included within the scope of the present invention.

Representative salts of the compounds of this invention include the conventional non-toxic salts and the quaternary ammonium salts that are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, trifluoro acetate and undecanoate. The term acid addition salts also comprises the hydrates and the solvent addition forms which the compounds of this invention are able to form. Examples of such forms are, for example, hydrates, alcoholates and the like.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates including dimethyl, diethyl, and dibutyl sulfate; and diamyl sulfates, long chain halides such as decyl, lauryl, myristyl and strearyl chlorides, bromides and iodides, aralkyl halides including benzyl and phenethyl bromides, and others.

The esters of appropriate compounds of this invention are pharmaceutically acceptable esters such as alkyl esters, including methyl, ethyl, propyl, isopropyl, butyl, isobutyl or pentyl esters, and the like. Additional esters such as phenyl-(C₁-C₅) alkyl may be used, although methyl ester is preferred.

Unless the context clearly indicates to the contrary, whenever the term “compounds of this invention,” “compounds of the present invention”, and the like, are used herein, they are intended to include the chemically feasible pharmaceutically acceptable salts and/or esters as well as all stereoisomeric forms of the referenced compounds.

### Method for the Preparation of the Compounds of the Present Invention

In general, the compounds of Formula (I) of this invention may be prepared by standard techniques known in the art, by known processes analogous thereto, and/or by processes disclosed below, using starting materials which are either commercially available, producible according to routine, conventional chemical methods or the synthesis of which is described herein. The particular process to be utilized in the preparation of a compound of this invention depends upon the specific compound desired. Such factors as whether the amine is substituted or not, the selection of the specific substituents possible at various locations on the molecule, and the like, each play a role in the path to be followed. Those factors are readily recognized by one of ordinary skill in the art.

The benzofuran and benzothiophene derivatives of Formula (I) are generally prepared by, but not limited to, the methods outlined below in Reaction Schemes 1 and 2.

In Reaction Scheme 1, the benzofuran or benzothiophene final product of Formula (I) where R¹ is H may be synthesized directly by the condensation of a properly substituted 2-cyanophenol (formula (II), where X═O) or 2-cyanothiophenol (formula (II), where X═S) with an appropriate 1-aryl-2-haloethanone of formula (E). The reaction is generally facilitated by a base, such as cesium carbonate, potassium carbonate, sodium carbonate or DBU, in a solvent such as DMF or MeCN, and at temperatures between room temperature to 100° C. to give the final product of formula (I).

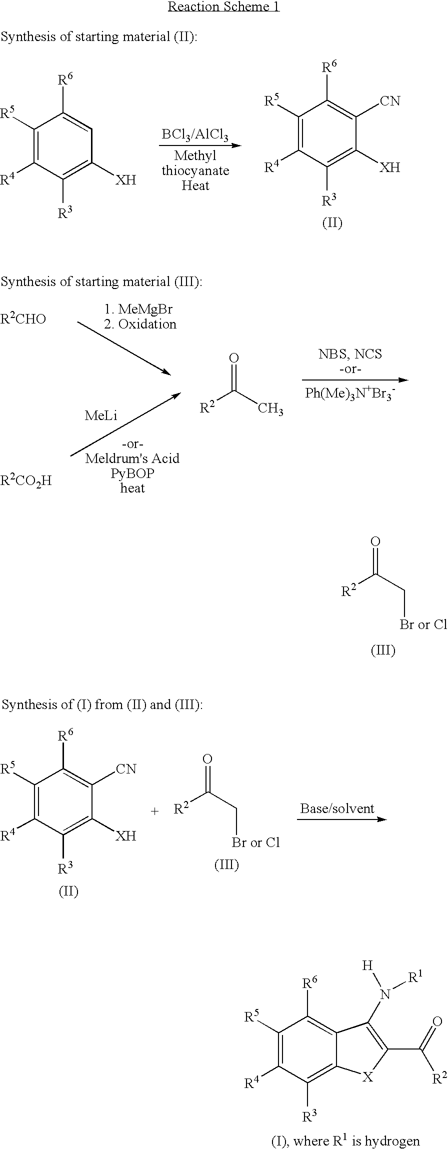

Alternatively, when the starting phenol or thiophenol is not readily available, the method outlined in Reaction Scheme 2 may be used, wherein intermediate benzofurans and benzothiophenes of formulas (IV) or (V) are first prepared in analogous manner and then converted to the final product (I) by Suzuki coupling reactions. Thus the halobenzofuran or halobenzothiophene (IV) is either allowed to react with a boronate ester of formula (VI) in the presence of a Pd catalyst and base, or it is converted to the boronate ester of formula (V) then coupled with the halo compound of formula (VII) under similar conditions. Starting materials (II), (III) (VI) and (VII) are generally commercially available or prepared by standard means known in the art and illustrated below in the preparative examples.

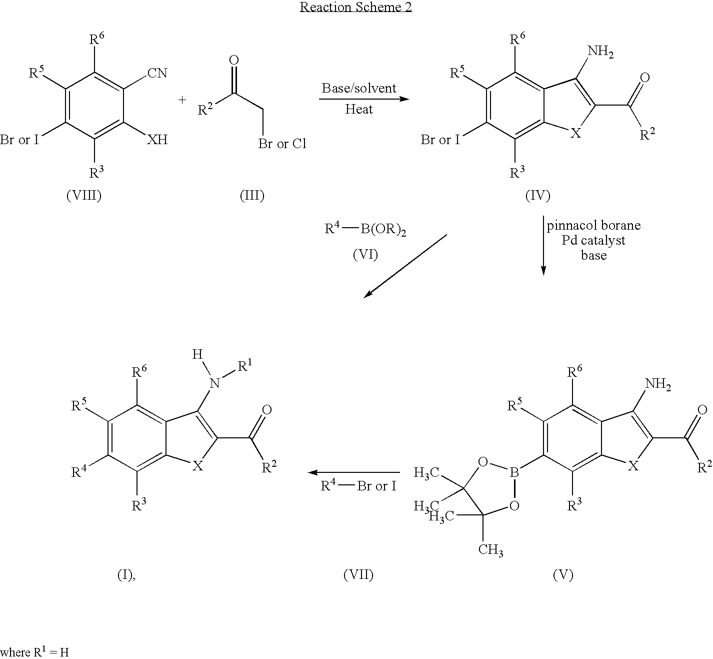

Compounds of formula (I) where R¹ is H, prepared by either Reaction Scheme 1 or Reaction Scheme 2 may be converted to other formula (I) compounds where R¹ is other than H, as shown in Reaction Scheme 3. For example, treatment with a base and an alkylating agent such as methyl iodide or methyl sulfate provides formula (I) compounds where R¹ is alkyl. Similarly, treatment with a base and an acylating agent such as acetyl chloride or benzoyl chloride gives compounds of formula (I) where R¹ is acetyl or benzoyl.

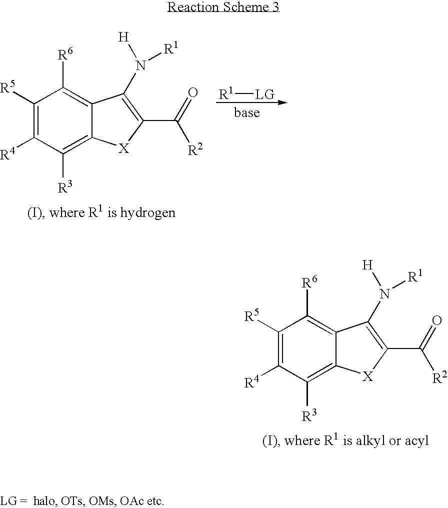

It is to be understood that sensitive or reactive substituents attached to intermediates or to compounds of formula I may need to be protected and deprotected during the preparations described above. Protecting groups in general may be added and removed by conventional methods well known in the art [see, e.g., T. W. Greene and P. G. M. Wuts, Protective Groups in Organic Synthesis; Wiley: New York, (1999)].

Variations of the compounds of the invention can be readily prepared using the processes described above and referenced below, or by other standard chemical processes known in the art, by employing appropriate starting materials or intermediate compounds that are readily available and/or are described herein.

Generally, a desired salt of a compound of this invention can be prepared in situ during the final isolation and purification of a compound by means well known in the art. For example, a desired salt can be prepared by separately reacting the purified compound in its free base or free acid form with a suitable organic or inorganic acid, or suitable organic or inorganic base, respectively, and isolating the salt thus formed. In the case of basic compounds, for example, the free base is treated with anhydrous HCl in a suitable solvent such as THF, and the salt isolated as a hydrochloride salt. In the case of acidic compounds, the salts may be obtained, for example, by treatment of the free acid with anhydrous ammonia in a suitable solvent such as ether and subsequent isolation of the ammonium salt. These methods are conventional and would be readily apparent to one skilled in the art.

The compounds of this invention may be esterified by a variety of conventional procedures including reacting the appropriate anhydride, carboxylic acid or acid chloride with the alcohol group of a compound of this invention. The appropriate anhydride is reacted with the alcohol in the presence of a base to facilitate acylation such as 1,8-bis[dimethylamino]naphthalene or N,N-dimethylaminopyridine. Or, an appropriate carboxylic acid can be reacted with the alcohol in the presence of a dehydrating agent such as dicyclohexylcarbodiimide, 1-[3-dimethylaminopropyl]-3-ethylcarbodiimide or other water soluble dehydrating agents which are used to drive the reaction by the removal of water, and, optionally, an acylation catalyst. Esterification can also be effected using the appropriate carboxylic acid in the presence of trifluoroacetic anhydride and, optionally, pyridine, or in the presence of N,N-carbonyldiimidazole with pyridine. Reaction of an acid chloride with the alcohol can be carried out with an acylation catalyst such as 4-DMAP or pyridine.

One skilled in the art would readily know how to successfully carry out these as well as other known methods of esterification of alcohols.

The purification of isomers and the separation of isomeric mixtures of a compound of formula (I) may be accomplished by standard techniques known in the art.

The compound of formula (IX) is prepared by a reaction between a compound of formula (X) and a compound of formula (XI)

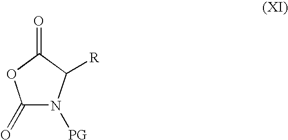

followed by a deprotection of the protective group PG,
wherein
R is defined by the side chain of the alpha amino acid which might carry further protective groups as defined above in the compound of the formula (IX), and
PG is a protective group selected from tert-butoxycarbonyl (Boc) and benzyloxycarbonyl (Z).

Preferably the protective group PG is the tert-butoxycarbonyl (Boc) group.

Surprisingly the reaction between compounds of formula (X) and compounds of formula (XI) according to the invention is selective regarding the acylation of the sulfonamid function and the amino function located at the benzofuran, i.e. only the sulfonamid function is acetylated getting compounds of formula (IX) selectively.

The reaction is generally facilitated by a base, preferably by 4-(N,N-dimethylamino)-pyridine (DMAP), in organic solvent and at temperatures between room temperature to the reflux temperature of the solvent, preferably between room temperature to 50° C., within 2 h and 20 h to give the PG protected intermediate. The reaction can also be carried out with ultrasonic conditions.

Organic solvent include but is not limited to dichloromethane, tetrahydrofurane, dimethyl sulfoxide and dimethylformamide. Preference is given to dimethylformamide or dichloromethane.

The intermediate can be isolated and purified by standard procedures e.g. flash chromatography.

Stereoisomers of the PG protected intermediates can be separated by standard procedures e.g. crystallization or chiral HPLC.

The deprotection of the PG group of the protected intermediate can be achieved by standard procedures as described e.g. in T. W. Greene and P. G. M. Wuts, Protective Groups in Organic Synthesis; Wiley: New York, (1999).

Examples of standard procedures for the deprotection of Boc- or Z-protected amines include, but are not limited to, reaction with an appropriate acid such as HCl, HBr, trifluoroacetic acid or methanesulfonic acid in an appropriate solvent such as dioxane, dichloromethane, tetrahydrofuran or acetic acid.

Further protecting groups in the side chains R might also be also cleaved under the conditions for removal of PG or they may be maintained in the molecule. If required, they may be removed in a separate deprotection step either prior to or after removal of PG.

Stereoisomers of the compounds of formula (IX) can be separated by standard procedures e.g. crystallization or chiral HPLC.

Compounds of the formula (XI) (Urethane protected N-Carboxy anhydrides) are commercially available or are prepared starting from appropriately protected amino acid derivatives according to literature procedures (M. Johnston et al. J. Org. Chem. 1985, 50, 2200; W. D. Fuller et al. J. Am. Chem. Soc. 1990, 112, 7414; S. Mobasheri et al. J. Org. Chem. 1992, 57, 2755).

Compounds of formula (XI) can be employed either as pure enantiomers or diastereoisomers or as enantiomeric or diastereomeric mixtures.

Generally, a desired salt of a compound of this invention can be prepared in situ during the final isolation and purification of a compound by means well known in the art. It may also in situ be formed during the final deprotection step by the acids or bases employed. For example, a desired salt can be prepared by separately reacting the purified compound in its free base or free acid form with a suitable organic or inorganic acid, or suitable organic or inorganic base, respectively, and isolating the salt thus formed. In the case of basic compounds, for example, the free base is treated with anhydrous HCl in a suitable solvent such as THF, and the salt isolated as a hydrochloride salt.

In the case of acidic compounds, the salts may be obtained, for example, by treatment of the free acid with anhydrous ammonia in a suitable solvent such as ether and subsequent isolation of the ammonium salt. These methods are conventional and would be readily apparent to one skilled in the art.

The purification of isomers and the separation of isomeric mixtures of a compound of formula (IX) may be accomplished by standard techniques known in the art.

Compositions Useful for the Method of this Invention

A compound of the present invention is useful in this method for treating the conditions described further herein when it is formulated as a pharmaceutically acceptable composition. A pharmaceutically acceptable composition is a compound of Formula (I) or (IX) in admixture with a pharmaceutically acceptable carrier. A pharmaceutically acceptable carrier is any carrier that is relatively non-toxic and innocuous to a patient at concentrations consistent with effective activity of the active ingredient so that any side effects ascribable to the carrier do not vitiate the beneficial effects of the active ingredient.

Commonly used pharmaceutical ingredients which can be used as appropriate to formulate the composition for its intended route of administration include: acidifying agents (examples include but are not limited to acetic acid, citric acid, fumaric acid, hydrochloric acid, nitric acid); alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine); adsorbents (examples include but are not limited to powdered cellulose and activated charcoal); aerosol propellants (examples include but are not limited to carbon dioxide, CCl₂F₂, F₂ClC—CClF₂ and CClF₃); air displacement agents (examples include but are not limited to nitrogen and argon); antifungal preservatives (examples include but are not limited to benzoic acid, butylparaben, ethylparaben, methylparaben, propylparaben, sodium benzoate); antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal); antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (examples include but are not limited to block polymers, natural and synthetic rubber, polyacrylates, polyurethanes, silicones, polysiloxanes and styrene-butadiene copolymers); buffering agents (examples include but are not limited to potassium metaphosphate, dipotassium phosphate, sodium acetate, sodium citrate anhydrous and sodium citrate dihydrate); carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (examples include but are not limited to edetate disodium and edetic acid); colorants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (examples include but are not limited to bentonite); emulsifying agents (examples include but are not limited to acacia, cetomacrogol, cetyl alcohol, glyceryl monostearate, lecithin, sorbitan monooleate, polyoxyethylene 50 monostearate); encapsulating agents (examples include but are not limited to gelatin and cellulose acetate phthalate); flavorants (examples include but are not limited to anise oil, cinnamon oil, cocoa, menthol, orange oil, peppermint oil and vanillin); humectants (examples include but are not limited to glycerol, propylene glycol and sorbitol); levigating agents (examples include but are not limited to mineral oil and glycerin); oils (examples include but are not limited to arachis oil, mineral oil, olive oil, peanut oil, sesame oil and vegetable oil); ointment bases (examples include but are not limited to lanolin, hydrophilic ointment, polyethylene glycol ointment, petrolatum, hydrophilic petrolatum, white ointment, yellow ointment, and rose water ointment); penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (examples include but are not limited to diethyl phthalate and glycerol); solvents (examples include but are not limited to ethanol, corn oil, cottonseed oil, glycerol, isopropanol, mineral oil, oleic acid, peanut oil, purified water, water for injection, sterile water for injection and sterile water for irrigation); stiffening agents (examples include but are not limited to cetyl alcohol, cetyl esters wax, microcrystalline wax, paraffin, stearyl alcohol, white wax and yellow wax); suppository bases (examples include but are not limited to cocoa butter and polyethylene glycols (mixtures); surfactants (examples include but are not limited to benzalkonium chloride, nonoxynol 10, oxtoxynol 9, polysorbate 80, sodium lauryl sulfate and sorbitan mono-palmitate); suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening agents (examples include but are not limited to aspartame, dextrose, glycerol, mannitol, propylene glycol, saccharin sodium, sorbitol and sucrose); tablet anti-adherents (examples include but are not limited to magnesium stearate and talc); tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch); tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (examples include but are not limited to dibasic calcium phosphate); tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch); tablet glidants (examples include but are not limited to colloidal silica, corn starch and talc); tablet lubricants (examples include but are not limited to calcium stearate, magnesium stearate, mineral oil, stearic acid and zinc stearate); tablet/capsule opaquants (examples include but are not limited to titanium dioxide); tablet polishing agents (examples include but are not limited to camuba wax and white wax); thickening agents (examples include but are not limited to beeswax, cetyl alcohol and paraffin); tonicity agents (examples include but are not limited to dextrose and sodium chloride); viscosity increasing agents (examples include but are not limited to alginic acid, bentonite, carbomers, carboxymethylcellulose sodium, methylcellulose, polyvinyl pyrrolidone, sodium alginate and tragacanth); and wetting agents (examples include but are not limited to heptadecaethylene oxycetanol, lecithins, sorbitol monooleate, polyoxyethylene sorbitol monooleate, and polyoxyethylene stearate).

The compounds of the present invention can be administered with pharmaceutically-acceptable carriers well known in the art using any effective conventional dosage unit forms formulated as immediate, slow or timed release preparations, including, for example, the following.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions.

Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations which are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

### Pharmaceutical Compositions According to the Present Invention can be Further Illustrated as Follows:

Sterile IV Solution: A 5 mg/mL solution of the desired compound of this invention is made using sterile, injectable water, and the pH is adjusted if necessary. The solution is diluted for administration to 1-2 mg/mL with sterile 5% dextrose and is administered as an IV infusion over 60 min.
Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 min.
Intramuscular suspension: The following solution or suspension can be prepared, for intramuscular injection:
50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol
Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
Tablets: A large number of tablets are prepared by conventional procedures so that the dosage unit was 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.
Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Preference is given to an aqueous formulation, for example a solution or suspension, comprising a compound of formula (I) for i.v. application.

### Method of Treating Cancer

The compounds and compositions described herein can be used to treat or prevent cancers of the central nervous system.

Examples of cancers of the central nervous system include but are not limited to astrocytomas (differentiated and anaplastic), gliomas, gangliomas, pilocytic astrocytomas, oligodendrogliomas (differentiated and anaplastic), ependymomas, glioblastomas, multiforme, mixed gliomas, oligoastrocytomas, medulloblastomas, retinoblastomas, neurinomas (neurilemmoma), neurofibromas (Schwannoma), neuroblastomas, pituitary adenomas, meningiomas, heamangioblastomas, tumors of the plexus chorioideus and brain metastases of other tumors.

Preference is given to the treatment or prevention of gliomas, neuroblastomas and/or glioblastomas.

An effective amount of a compound or composition of this invention can be administered to a patient in need thereof in order to achieve a desired pharmacological effect. A patient, for the purpose of this invention, is a mammal, including a human, in need of treatment (including prophylactic treatment) for a particular disorder described further herein. A pharmaceutically effective amount of compound or composition is that amount which produces a desired result or exerts an influence on the particular cancer of the central nervous system being treated.

Surprisingly the compounds and compositions described herein, including salts and esters thereof, inhibit the growth of various neurologically relevant cancer cells.

### Dosage

Based upon standard laboratory techniques known to evaluate compounds useful for the prevention and/or treatment of the diseases or disorders described above by standard toxicity tests and by standard pharmacological assays for the determination of the prevention and/or treatment of the conditions identified above in mammals, the effective dosage of the compounds of this invention can readily be determined for prevention and/or treatment of each desired indication. The amount of the active ingredient to be administered in the prevention and/or treatment of one of these conditions can vary widely according to such considerations as the particular compound and dosage unit employed, the mode of administration, the duration of treatment (including prophylactic treatment), the age and sex of the patient treated, and the nature and extent of the condition to be prevented and/or treated.

The total amount of the active ingredient to be administered will generally range from about 0.001 mg/kg to about 300 mg/kg, and preferably from about 0.10 mg/kg to about 150 mg/kg body weight per day. A unit dosage may contain from about 0.5 mg to about 1500 mg of active ingredient, and can be administered one or more times per day. The daily dosage for administration by injection, including intravenous, intramuscular, subcutaneous and parenteral injections, and use of infusion techniques will preferably be from 0.01 to 200 mg/kg of total body weight. The daily rectal dosage regimen will preferably be from 0.01 to 200 mg/kg of total body weight. The daily vaginal dosage regimen will preferably be from 0.01 to 200 mg/kg of total body weight. The daily topical dosage regimen will preferably be from 0.1 to 200 mg administered between one to four times daily. The transdermal concentration will preferably be that required to maintain a daily dose of from 0.01 to 200 mg/kg. The daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of administration and number of doses of a compound or composition of the present invention or a pharmaceutically acceptable salt or ester thereof can be ascertained by those skilled in the art using conventional prevention and/or treatment tests.

The compounds of this invention can be administered as the sole pharmaceutical agent or in combination with one or more other pharmaceutical agents where the combination causes no unacceptable adverse effects. For example, the compounds of this invention can be combined with other anti-hyper-proliferative or other indication agents, and the like, as well as with admixtures and combinations thereof.

For example, optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclo-phosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment and/or prevention of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyladenine, ethinyl estradiol, 5-fluorodeoxyuridine, 5-fluorodeoxy-uridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of this invention include but are not limited to other anti-cancer agents such as epothilone, irinotecan, raloxifen, topotecan, asparaginase, bleomycin, carboplatin, carmustine, chlorambucil, cisplatin, colaspase, cyclophosphamide, cytarabine, dacarbazine, dactinomycin, daunorubicin, doxorubicin (adriamycin), epirubicin, etoposide, 5-fluorouracil, hexamethylmelamine, hydroxyurea, ifosfamide, irinotecan, leucovorin, lomustine, mechlorethamine, 6-mercaptopurine, mesna, methothrexate, mitomycinC, mitoxantrone, prednisolone, prednisone, procarbazine, raloxifen, streptozocin, tamoxifen, thioguanine, topotecan, vinblastine, vincristine, vindesine, amnogluthethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentstatin, PALA, plicamycin, semustine, teniposide, testosterone propionate, thiotepa, trimethylmelamine, uridine, and vinorelbine, oxaliplatin, gemcitabine, capecitabine, epothilone and its natural or synthetic derivatives, tositumomab, trabedectin, and temozolomide, trastuzumab, cetuximab, bevacizumab, pertuzumab, ZD-1839 (Iressa), OSI-774 Tarceva), CI-1033, GW-2016, CP-724,714, HKI-272, EKB-569, STI-571 (Gleevec), PTK-787, SU-11248, ZD-6474, AG-13736, KRN-951, CP-547, 632, CP-673,451 and sorafenib.

Other compounds useful in combination with the compounds of the present invention are anti-epileptic agents, agents acting against brain edemas, analgesics (e.g. ibuprufen, acetyl salicylic acid, naproxen, acetaminophene, Cox-2 inhibitors), antidepressants (for instance selective serotonin inhibitors or tricyclic antidepressants, anticonvulsiva, capsicaine, mexiletine) or immunomodulating agents (e.g. Parapoxvirus ovis base agent or an interferon).

### PREPARATIVE EXAMPLES OF THE INVENTION

Proton (¹H) nuclear magnetic resonance (NMR) spectra were measured with a General Electric GN-Omega 300 (300 MHz) spectrometer with either Me₄Si (δ 0.00) or residual protonated solvent (CHCl₃ δ 7.26; MeOH δ 3.30; DMSO δ 2.49) as standard. Carbon (¹³C) NMR spectra were measured with a General Electric GN-Omega 300 (75 MHz) spectrometer with solvent (CDCl₃ δ 77.0; d₃-MeOD; δ 49.0; d₆-DMSO δ 39.5) as standard.

Chiral separations were performed using a commercially available Chiracel® AD HPLC column, eluting with a gradient of isopropanol in hexane (from 1% to 15%) with addition of 0.1% trifluoroacetic acid.

### LC-MS- and HPLC-Methods:

### Method 1 (Standard Conditions for Preparative HPLC):

Column: VP 250/21Nukleodur 100-5, C18 ec, Macherey&Nagel: 762002;

Eluent A: 0.01% Trifluoroacetic acid in water, Eluent B: Acetonitrile/0.01% Trifluoroacetic acid; Gradient: 0 min 0% B, 20 min 20% B, 40 min 20% B, 60 min 30% B, 100 min 30% B, 110 min 100% B, 132 min 100% B; Flow: 5 ml/min; 30° C.; UV-Detection: 210 or 257 nm

### Method 2 (Standard Conditions for Analytical HPLC):

Column: XTerra 3.9×150 WAT 186000478; Temperature: RT; Flow: 1 ml/min;

Eluent A: 10 ml 70% Perchloric acid in 2.5 l water, Eluent B: Acetonitrile, Gradient: 0.0 min 20% B, 1 min 20% B, 4 min 90% B, 9 min 90% B, 10 min 20% B.

### Method 3 (Standard Conditions for LC-MS):

Instrument: Micromass LCT with HPLC Agilent Serie 1100;

Column: Waters Symmetry C18; 3.5 μm; 50 mm×2.1 mm;

Eluent A: 1 l water+1 ml 98-100% formic acid; Eluent B: 1 l acetonitrile+1 ml 98-100% formic acid; Gradient: 0 min 100% A, 1 min 100% A, 6 min 10% A, 8 min 0% A, 10 min 0% A, 10.1 min 100% A, 2 min 100% A; Flow 0 min-10 min 0.5 ml/min, 10.1 min 1 ml/min, 12 min 0.5 ml/min; 40° C.; UV-Detection DAD: 208-500 nm.

### Method 4: (Standard Conditions for LC-MS):

Instrument: Micromass ZQ; Type HPLC: HP 1100 Series; UV DAD; Column: Phenomenex Synergi 2μ Hydro-RP Mercury 20 mm×4 mm; Eluent A: 1 l Water+0.5 ml 50% formic acid, Eluent B: 1 l Acetonitrile+0.5 ml 50% formic acid; Gradient: 0.0 min 90% A, 2.5 min 30% A, 3.0 min 5% A, 4.5 min 5% A; Flow 0.0 min 1 ml/min, 2.5 min/3.0 min/4.5 min. 2 ml/min; 50° C.; UV-Detection: 210 nm.

### Method 5: (Standard Conditions for LC-MS):

Instrument: Micromass Quattro LCZ mit HPLC Agilent Series 1100; Column: Phenomenex Synergi 2μ Hydro-RP Mercury 20 mm×4 mm; Eluent A: 1 l Water+0.5 ml 50% formic acid, Eluent B: 1 l Acetonitrile+0.5 ml 50% ige formic acid; Gradient: 0.0 min 90% A, 2.5 min 30% A, 3.0 min 5% A, 4.5 min 5% A; Flow: 0.0 min 1 ml/min, 2.5 min/3.0 min/4.5 min 2 ml/min; 50° C.; UV-Detection: 208-400 nm.

### Method A1 (Chiral HPLC; Analytical):

Chiral silicagel phase SYFO 5326 (250 mm×4.6 mm) based upon Poly(N-methacryloyl-L-leucine-dicyclopropylmethylamide); i-Hexane/ethyl acetate 35:65 (vol/vol); Temperature: 24° C.; Flow: 2 ml/min; UV-Detection: 270 nm.

### Method B1 (Chiral HPLC; Analytical):

Chiral silicagel phase SYFO 7490 (250 mm×4.6 mm) based upon Poly(N-methacryloyl-L-leucine-tert-butylamide); i-Hexane/ethyl acetate 35:65 (vol/vol); Temperature: 24° C.; Flow: 2 ml/min; UV-Detection: 270 nm.

### Method C1 (Chiral HPLC; Analytical):

Chiral silicagel phase SYFO 7490 (250 mm×4.6 mm) based upon Poly(N-methacryloyl-L-leucine-tert-butylamide); i-Hexane/ethyl acetate 65:35 (vol/vol); Temperature: 24° C.; Flow: 2 ml/min; UV-Detection: 270 nm.

### Method A2 (Chiral HPLC; Preparative):

Chiral silicagel phase SYFO 5326 (670 mm×40 mm) based upon Poly(N-methacryloyl-L-leucine-dicyclopropylmethylamide); i-Hexane/ethyl acetate 25:75 (vol/vol); Temperature: 24° C.; Flow: 80 ml/min; UV-Detection: 270 nm.

### Method B2 (Chiral HPLC; Preparative):

Chiral silicagel phase SYFO 7490 (670 mm×40 mm) based upon Poly(N-methacryloyl-L-leucine-tert-butylamide); i-Hexane/ethyl acetate 65:35 (vol/vol); Temperature: 24° C.; Flow: 50 ml/min; UV-Detection: 260 nm.

### NMR:

NMR-measurements were performed at a Bruker DMX500 with a proton frequency of 500, 13 MHz. Samples were dissolved in DMSO-d6, temperature 302K.

### ABBREVIATIONS

When the following abbreviations are used herein, they have the following meaning:
ADDP 1,1′ (azodicarbonyl)-dipiperidine
DBU 1,8-Diazabicyclo [5.4.0]undec-7-ene
DMF N,N-dimethylformamide
DMSO Dimethylsulfoxide
EA Elemental analysis
ES Electrospray
Et Ethyl
Et₂O Diethyl ether
EtOAc Ethyl acetate
GC-MS Gas chromatography-mass spectroscopy
HEX Hexanes
LC-MS Liquid Chromatography/Mass Spectroscopy
Me Methyl
MeCN Acetonitrile
MeOH Methanol
MPLC Medium Pressure Liquid Chromatograph
NCS N-chlorosuccinimide
NMR Nuclear Magnetic Resonance Spectroscopy
Pd(dppf)₂Cl_{2 [}1,1′-bis(diphenylphosphino)-ferrocene]dichloro-palladium(II)
Ph Phenyl
PyBOP Benzotriazole-1-yl-oxy-tris-pyrrolidino-phosphonium hexafluorophosphate
   RT Retention time (HPLC)
R_{f} TLC Retention Factor
rt Room temperature
THF Tetrahydrofuran
TLC Thin layer chromatography

### Preparation of Starting Materials and Intermediates

General Method A: 2-Halo-1-arylketones (III)

Compounds of formula (III) are either commercially available or may be prepared as shown in the Reaction Scheme for General Method A, and as described in one or more of the Examples below.

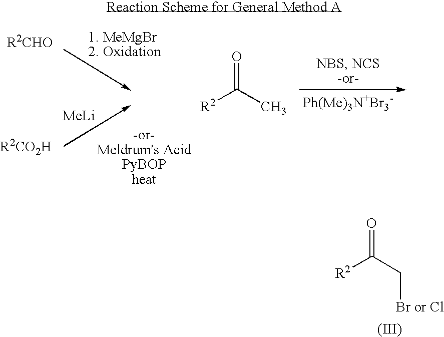

### Example 1

### Method A-1

### Preparation 2-bromo-1-(2,4,6-trichlorophenyl)ethanone

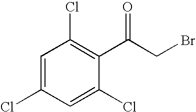

A mixture of 1,3,5-trichlorobenzene (10.0 g, 55.1 mmol), 2-bromoacetyl bromide (5.0 mL, 57.8 mmol, 1.05 eq), and aluminum chloride (7.7 g, 57.8 mmol, 1.05 eq) was heated neat at 80° C. under argon for 17 h until a black precipitate is formed. The reaction was cooled to room temperature, and the resultant black mass was dissolved in ethyl acetate (500 mL). Water (200 mL) was added slowly at 0° C. to quench the reaction, and the biphasic layers were separated. The organic layer was then washed with water (2×150 mL) and brine (1×150 mL), dried (MgSO₄), filtered, and evaporated in vacuo. Recrystallization from hexane gave 11.5 g (69.3%) of 2-bromo-1-(2,4,6-trichloropheny)ethanone as a fluffy white solid. ¹H-NMR (DMSO-d₆) δ 7.86 (s, 2H), 4.78 (s, 2H); R_{f}=0.28, 2% ethyl acetate-hexane.

### Example 2

### Method A-2a

### Preparation of 2-bromo-1-(2,5-dichlorophenyl)ethanone

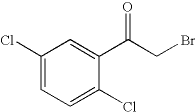

To 2,5-dichloroacetophenone (5.0 g, 26.45 mmol) in anhydrous tetrahydrofuran (53 mL) under argon was added phenyltrimethylammonium tribromide (9.94 g, 26.45 mmol, 1.0 eq) at 0° C. The reaction mixture was stirred at ambient temperature for 16 h, concentrated, and re-dissolved in ethyl acetate. The organic layer was washed with water (2×250 mL) and brine (1×150 mL), dried (MgSO₄), filtered, and evaporated in vacuo. Purification using MPLC chromatography (Biotage) gave 3.47 g (52.5%) of 2-bromo-1-(2,5-dichloropheny)ethanone as a clear oil. ¹H-NMR (DMSO-d₆) δ 7.93 (dd, J=2.1 Hz, 0.9 Hz, 1H), 7.61 to 7.60 (m, 2H), 4.86 (s, 2H).

### Example 3

### Method A-2b

### Preparation of 2-Bromo-1-(2-bromo-4-fluoro-phenyl)-ethanone

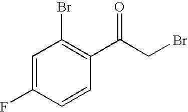

This compound was prepared from 1-(2-bromo-4-fluoro-phenyl)-ethanone (2.5 g, 11.52 mmol) in the manner described for 2-bromo-1-(2,5-dichlorophenyl)-ethanone (Example A-2), affording 2.14 g (63%) of 2-bromo-1-(2-bromo-4-fluoro-phenyl)-ethanone as a clear oil. ¹H-NMR (CD₂Cl₂) [symbol] 7.57 (dd, J=9.6 Hz, 1H), 7.44 (dd, J=8, 2 Hz, 1H), 7.21 (m, 7.21-7.14, 1H), 4.51 (s, 2H); TLC R_{f}=0.38, 15% ethyl acetate hexanes.

### Example 4

### Method A-3

### Preparation of 2-Chloro-1-(4-methyl-3-pyridinyl)ethanone hydrochloride

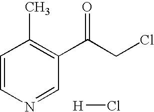

### Step 1: Preparation of 1-(4-methyl-3-pyridinyl)ethanone

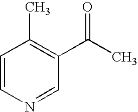

A solution of 3-acetylpyridine (100 g, 0.82 mol), dimethyl sulfide (400 mL, 5.4 mol) and copper (I) iodide (7.94 g, 0.041 mol) in anhydrous THF (2 L) was stirred at room temperature under an argon atmosphere. Phenyl chloroformate (0.4 mL, 0.82 mol) was then added, producing a dark brown precipitate. After 30 min, the mixture was cooled below −21° C. and methyl magnesium bromide (1.4 M in 3:1 toluene-THF, 586 mL, 0.82 mol) was added over 50 min, keeping the reaction temperature below −15° C. The color lightened as the mixture became a solution; a lime green precipitate formed near the end of the addition, but re-dissolved upon completion. The mixture was stirred and allowed to warm slowly; after 2 h it had warmed to 8.8° C. Saturated aqueous ammonium chloride solution (500 mL) was added; after stirring 10 min, the mixture was poured into a separatory funnel with water (500 mL). The organic phase was separated, washed with brine (500 mL), dried (Na₂SO₄), filtered and then concentrated in vacuo. The residue was purified by silica gel chromatography using a hexane-EtOAc gradient to afford 134.3 g (63.7%) of the intermediate dihydropyridine.

A solution of the intermediate dihydropyridine (0.52 mol) in dichloromethane (100 mL) was added to a stirred suspension of sulfur (16.67 g, 0.52 mol) in decalin and slowly heated to reflux under an argon sweep. After refluxing 1 h, the mixture was allowed to cool to room temperature, then filtered through a pad of silica gel. After eluting the decalin with hexane, elution with a hexane-diethyl ether gradient afforded 49.4 g (70.3%) the desired 1-(4-methyl-3-pyridinyl)ethanone as a reddish-brown oil: TLC R_{f} 0.19 (diethyl ether); TLC R_{f} 0.14 (1:1 hexane-EtOAc); ¹H NMR (CD₂Cl₂) [symbol] 8.9 (s, 1H), 8.5 (d, 1H), 7. [symbol] (dd, 1H), 2.6 (s, 3H), 2.51 (s, 3H); GC MS 135 (M⁺).

### Step 2: Preparation of 2-chloro-1-(4-methyl-3-pyridinyl)ethanone hydrochloride

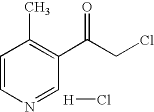

In a 500 mL round bottom flask was placed 1-(4-methyl-3-pyridinyl)ethanone (10.0 g, 74 l mmol) in 90 mL of Et₂O. To this solution was added 88.9 mL of 1M HCl/Et₂O (1.2 eq, 88.9 mmol) with stirring and the solution allowed to stir for 1 h at room temperature, at which point, the precipitate was filtered and washed with Et₂O. The solid was then dried in vacuo at about 60° C. This HCl salt (12. g, 70.0 mmol) was then dissolved in 70.0 mL of 1M HCl/acetic acid where 9.34 g (1 eq, 70.0 mmol) of N-chlorosuccinimide (NCS) was added and the reaction allowed to stir under Argon at room temperature overnight. At this point, 300 mL of Et₂O was added resulting in an off-white precipitate. This was allowed to stir for 1 h at which point the solid was filtered and rinsed with Et₂O to provide 12.0 g (83%) of the desired 2-chloro-1-(4-methyl-3-pyridinyl)ethanone hydrochloride. GC/MS RT=6.60 min; ¹H-NMR (DMSO-d₆) δ 2.51 (s, 3H), 5.15 (s, 2H), 7.68 (d, 1H), 8.68 (d, 1H), 9.06 (s, 1H); [M]⁺169 (95%).

### Example 5

### Method A-4

### Preparation of 2-chloro-1-[4-(trifluoromethyl)-3-pyridinyl]ethanone hydrochloride

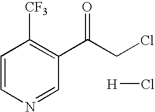

### Step 1

In a 250 mL round bottom flask was placed 3.0 g of 4-trifluoronicotinic acid (15.7 mmol, 1 eq) in 100 mL of THF. To this was added 5.3 mL (3.8 g, 37.7 mmol, 2.4 eq) of triethylamine and 9.8 g (18.8 mmol, 1.2 eq) of PyBOP. This was allowed to stir for 10 min at room temperature where 2.7 g of Meldrum's acid (18.8 mmol, 1.2 eq) was added and the reaction allowed to stir at room temperature overnight (18 h).

At this point, 30 mL of 1M HCl (aq) was added and the reaction turned immediately from orange to purple. This was then heated at for 18 h gradually turning from purple to yellow.

The reaction was then basified with saturated NaHCO₃ and extracted with EtOAc (3×200 mL). The combined organics were dried, filtered, and evaporated. The residue was purified via BIOTAGE (35% EtOAc/Hex) to provide methyl 4-trifluoromethylnicotinate 1.84 g (62%) of the desired product as a colorless oil. TLC R_{f}=0.57 (50% EtOAc:Hex).

### Step 2

In a 100 mL flask was placed 1.84 g (9.7 mmol, 1 eq) of methyl 4-trifluoromethylnicotinate in 25 mL of 1 M HCl in CH₃COOH. To this was then added 1.3 g of NCS (9.7 mmol, 1 eq) and the reaction allowed to stir overnight (18 h).

The mixture was then transferred to a 500 mL Erlenmeyer flask and to this was added 300 mL of 2 M HCl in Et₂O with stirring. This resulted in a white precipitate which was then filtered to provide 1.2 g (49%) of the desired 2-chloro-1-[4-(trifluoromethyl)-3-pyridinyl]ethanone hydrochloride as a white solid. ¹H-NMR (DMSO-d₆) δ 9.21 (s, 1H), 9.02 (d, 1H), 7.94 (d, 1H), 5.19 (s, 2H).

### Example 6

### Method A-5

### Preparation of 1-Benzo[1,3]dioxol-4-yl-2-bromo-ethanone

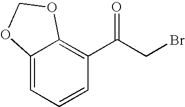

### Step 1: Preparation of Starting Material 1-Benzo-[1,3]-dioxol-4-yl-ethanone

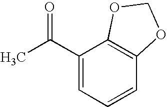

To a solution of MeMgBr in THF (1 M, 50 mL, 50 mmol, 1.5 eq) was diluted with 50 mL THF and cooled to −10 C. A solution of benzo[1,3]dioxole-4-carboxaldehyde (5.0 g, 33.3 mmol) in 50 mL THF was slowly added, and the reaction left to stir for 1 h. The reaction mixture was then quenched by pouring into 500 mL of ice cold sat. ammonium chloride and the mixture extracted with ether. The organic layers were dried over sodium sulfate and filtered through a plug of silica gel before concentrating in vacuo, providing 4.9 g of a white solid. A mixture of this solid (2.0 g, 12.0 mmol) and MnO₂ (10.5 g, 120.4 mmol, 10.0 eq) in 75 mL diethyl ether was stirred vigorously for 48 h. The reaction mixture was then filtered first through a plug of silica gel, then through a 0.46 μm frit before concentrating in vacuo to provide 2.1 g of an off-white solid. Purification by MPLC (Biotage) using a hexane-ethyl acetate gradient provided 1.47 g (74%) of 1-benzo[1,3]dioxol-4-yl-ethanone as an off-white solid. ¹H-NMR (CDCl₃) δ 7.35 (d, J=8 Hz, 1H), 6.97 (dm, J=8 Hz, 1H), 6.87 (dd, J=8 Hz, 1H), 6.08 (s, 2 μl), 2.59 (s, 3H); TLC R_{f}=0.18, 25% ethyl acetate-hexanes.

### Step 2: Preparation of Intermediate 1-Benzo[1,3]-dioxol-4-yl-2-bromo-ethanone

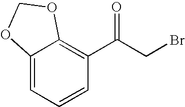

This compound was prepared from 1-benzo[1,3]dioxol-4-yl-ethanone (2.15 g, 13.1 mmol) in the manner described for 2-bromo-1-(2,5-dichlorophenyl)ethanone (Example 1-2), affording 1.54 g (48%) of 1-benzo[1,3]dioxol-4-yl-2-bromo-ethanone as an off-white solid. ¹H-NMR (CD₂Cl₂) ; 7.41 (dd, J=8.1 Hz, 1H), 7.05 (dd, J=8.1 Hz, 1H), 6.94 (dd, J=8.8 Hz, 1H), 6.13 (s, 2H), 4.55 (s, 2H). TLC R_{f}=0.28, 15%, ethyl acetate-hexanes.

### Example 7

### Method A-6

### Preparation of starting material 2-bromo-1-[3-(tert-butyl-diphenyl-silanyloxy)-phenyl]ethanone

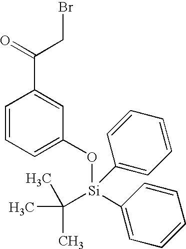

### Step 1: Preparation of 1-[3-(tert-butyl-diphenylsilanyloxy)phenyl]ethanone

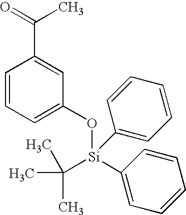

To 1-(3-hydroxy-phenyl)ethanone (3.3 g, 24.2 mmol) and tert-butylchlorodiphenyl-silane (7.3 g, 26.7 mmol, 1.1 eq) in anhydrous dichloromethane (50 mL) at 0° C. was added dimethylaminopyridine (296 mg, 2.42 mmol, 0.1 eq) and triethylamine (2.69 g, 26.7 mmol, 1.1 eq), and the reaction mixture was stirred at room temperature under argon for 16 h. The reaction mixture was diluted with ethyl acetate and water. The organic layer was washed with water, brine, and dried over magnesium sulfate. The solvent was evaporated under reduced pressure to afford 8.7 g (95.8%) of crude product. ¹H-NMR (Acetone-d₆) δ 7.78 (m, 5H), 7.56 to 7.38 (m, 7H), 7.22 (m, 1H), 7.00 (m, 1H), 2.38 (s, 3H), 1.12 (s, 9H); MS ES (MH⁺=375); R_{f}=0.90 (30% ethyl acetate-hexane).

### Step 2: Preparation of 2-bromo-1-[3-(tert-butyl-diphenyl-silanyloxy)phenyl]ethanone

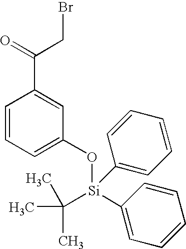

This compound was prepared from 1-[3-(tert-butyl-diphenyl-silanyloxy)phenyl]-ethanone (8.7 g, 23.23 mmol) in the manner described for 2-bromo-1-(2,5-dichlorophenyl)ethanone, affording 10.2 g (96.8%) of a clear oil. ¹H-NMR (Acetone-d₆) δ 7.78 (m, 4H), 7.60 (m, 1H), 7.50 to 7.40 (m, 7H), 7.22 (m, 1H), 7.05 (m, 1H), 4.56 (s, 2H), 1.13 (s, 9H); R_{f}=0.92 (30% ethyl acetate-hexane).

This material was used as the protected form in the synthesis of Example 104; desilylation occurred during the benzofuran-forming step.

### General Method H: Preparation of Intermediates (VI) and (VII)

The aryl halides (VII), the arylboronic acids, or the arylboronates (VI) used to prepare compounds in this invention of formula (I) (see Methods B, C, D, and G below) were either commercially available or prepared by one or more methods described in Examples below. Arylhalides (VII), prepared by the methods described hereafter, may subsequently be used either directly as starting materials for General Methods B, C-1, D-1, and D-3 described below, or converted to the corresponding boronates of formula (VI) using procedures described in step 1 of Examples C-2 and D-2 and used as described in General Method.

### Example 8

### Method H-1

### Preparation of 1-Bromo-3-methylsulfanylmethyl-benzene

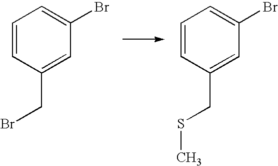

Sodium thiomethoxide (0.616 g, 8.8 mmol) was added to DMF (8 mL) and cooled to 0° C. To this solution was added 1-bromo-3-bromomethyl-benzene (2 g, 8 mmol). The mixture was allowed to warm to rt and stir for 18 h. The mixture was then poured into cold water (50 mL) and extracted with EtOAc (3×20 mL). The organics were combined and dried with sodium sulfate. The solution was concentrated in vacuo to yield the crude product, which was then purified via flash chromatography (5% ethyl acetate-hexanes) to yield 1.3 g (68.5%) of 1-bromo-3-methylsulfanylmethyl-benzene as a pure product. ¹H-NMR (methylene chloride-d2) δ 7.48-7.47 (m, 1H), 7.392 (dt, J=7.9, 1.5 Hz, 1H), 7.28-7.207 (m, 2H), 3.64 (s, 2H), 1.99 (s, 3H); LC-MS RT: 3.70, [M+H]⁺: 354.1.

### Example 9

### Method H-2

### Preparation of Alkyl Aryl Thioethers

### Preparation of 1-Bromo-3-isopropylsulfanyl-benzene

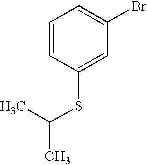

3-Bromobenzenethiol (1 g, 5.3 mmol) was added to acetone (25 mL). Next was added potassium carbonate (1.46 g, 10.58 mmol) and 2-iodopropane (1.17 g, 6.88 mmol). This was refluxed for 5 h. Reaction was then cooled to rt and filtered through a pad of Celite. The organic was then concentrated in vacuo and taken up in ether at which time a white precipitate crashed out. The organic was then re-filtered through the same celite plug and concentrated in vacuo to provide 1.14 g (93.17%) of 1-bromo-3-isopropylsulfanyl-benzene as an oil. ¹H-NMR (methylene chloride-d2) δ 7.54 (s, 1H), 7.37-7.31 (m, 2H), 7.18 (t, J=7.9 Hz, 1H), 3.50-3.36 (m, 1H), 1.31 (d, J=6.1 Hz, 6H); LC-MS RT: 4.15, [M+H]⁺: 233.2.

### Example 10

### Method H-3

### Preparation of 1-Bromo-3-methylsulfonyl-benzene

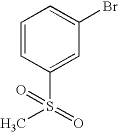

### Step 1: Preparation of 1-Bromo-3-methanesulfinyl-benzene

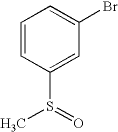

3-Bromothioanisol (0.5 g, 2.46 mmol) was added to methylene chloride (12 mL) and chilled to 0° C. To this was added 3-chloroperoxybenzoic acid (0.467 g, 2.71 mmol). The m-CPBA did not dissolve completely. The mixture was stirred overnight. The reaction was quenched with a saturated sodium thiosulfate (30 mL) solution. The product was extracted with EtOAc (3×20 mL). The organic fractions were combined, washed with brine (20 mL), and dried with sodium sulfate. The organic was then concentrated to yield 0.912 g (81%) 1-bromo-3-methanesulfinyl-benzene. ¹HNMR (methylene chloride-d₂) δ 7.83 (t, J=2.0 Hz, 1H), 7.66 (d, J=7.6 Hz, 1H), 7.57 (d, J=8.3 Hz, 1H), 7.44 (t, J=7.9 Hz, 1H), 2.77 (s, 3H); LC-MS RT: 1.28, [M+H]⁺: 219.0.

### Step 2: Preparation of 1-Bromo-3-methanesulfonyl-benzene

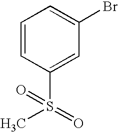

3-Bromothioanisol (8.7 g, 43 mmol) was added to methylene chloride (125 mL) chilled to 0° C. To this was added 3-chloroperoxybenzoic acid (22.2 g, 129 mmol). The m-CPBA did not dissolve completely. The mixture was stirred overnight. The reaction was quenched with a saturated sodium thiosulfate (150 mL) solution. The product was extracted with EtOAc (3×100 mL). The organic fractions were combined, washed with brine (75 mL), and dried with sodium sulfate. The organic was then concentrated to yield 9.89 g (97%) 1-bromo-3-methanesulfonyl-benzene. ¹HNMR (methylene chloride-d₂) δ 8.09 (s, 1H), 7.85 (dd, J=19.2, 7.8 Hz, 2H), 7.50 (t, J=8.2 Hz, 1H), 3.06 (s, 3H); GC-MS RT: 6.49, [M+H]⁺: 236.0.

### Example 11

### Method H-4

### Preparation of N-(3-Iodo-benzyl)-methanesulfonamide

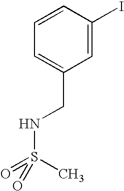

A mixture of 3-iodobenzylamine (1.0 g, 4.29 mmol) and methanesulfonyl chloride (0.35 mL, 4.51 mmol, 1.05 eq) in anhydrous pyridine (2.1 mL) was stirred at 50° C. under argon for 3 days. The cooled reaction was quenched with 1N HCl and diluted with ethyl acetate. The ethyl acetate layer was washed with water, brine, and dried over sodium sulfate. The solvent was removed at reduced pressure, and the crude product was purified on the MPLC (Biotage) eluted with 25% ethyl acetate-hexane. Crystallization from dichloromethane-ether-hexane afforded 1.307 g (97.9%) of the product. ¹H-NMR (DMSO-d₆) [symbol] 7.68 (s, 1H), 7.60 (ddd, J=7.8 Hz, 1.8 Hz, 1.2 Hz, 1H), 7.55 (t, J=6.3 Hz, 1H), 7.32 (d, J=9.0 Hz, 1H), 7.80 (t, J=7.8 Hz, 1H), 4.09 (d, J=6.6 Hz, 2H), 2.85 (d, J=1.8 Hz, 3H)δ LC-MS (ES MH⁺=264, R_{f}=2.39 min); R_{f}=0.48 (50% ethyl acetate-hexane).

### Example 12

### Method H-5

### Preparation of 1-(3-Iodo-phenyl)-3-methyl-urea

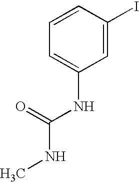

A mixture of 3-iodoaniline (1.0 g, 4.57 mmol) and methylisocyanate (0.29 mL, 5.02 mmol, 1.1 eq) in anhydrous N,N-dimethylformamide (3.0 mL) was stirred at 100° C. under argon for 16 h. The reaction was diluted with ethyl acetate and washed with water, brine, and dried over sodium sulfate. The solvent was removed at reduced pressure, and the crude oil was crystallized from ether-hexane to afford 732.5 mg (58.1%) of the product. ¹H-NMR (DMSO-d₆) δ 8.60 (s, 1H), 7.93 (t, J=1.8 Hz, 1H), 7.25 (ddd, J=8.1 Hz, 2.1 Hz, 0.9 Hz, 1H), 7.20 (ddd, J=8.1 Hz, 2.1 Hz, 0.9 Hz, 1H), 6.98 (t, J=8.1 Hz, 1H), 6.04 (broad d, J=4.8 Hz, 1H), 2.60 (d, J=5.4 Hz, 3H); R_{f}=0.23 (50% ethyl acetate-hexane).

### Example 13

### Method H-6

### Preparation of (R)-3-(3-Bromo-phenoxy)-propane-1,2-dioll

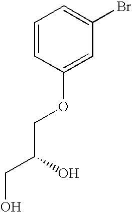

To 3-bromophenol (1.0 g, 5.78 mmol) and (R)-(+)-glycidol (428 mg, 5.78 mmol, 1.0 eq) in ethanol (50 mL) was added triethylamine (29 mg, 0.29 mmol, 0.05 eq), and the reaction mixture was refluxed under argon for 3 h. The reaction mixture was cooled and poured into ethyl acetate and water. The organic layer was washed with water, brine, and dried over sodium sulfate. The solvent was removed at reduced pressure, and the crude product was purified on the MPLC (Biotage) eluted with 30% ethyl acetate-hexane to give the diol as a white solid (1.20 g, 84.0%). ¹H-NMR (Acetone-d₆) δ 7.23 (t, J=8.4 Hz, 1H), 7.11 (m, 2H), 6.95 (m, 1H), 4.12 (m, 2H), 3.98 (m, 2H), 3.80 (m, 1H), 3.65 (m, 2H); R_{f}=0.12 (30% ethyl acetate-hexane).

### Example 14

### Method H-7

### Preparation of 2-fluoro-3-iodo-pyridine

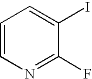

To a solution of n-butyllithium in hexanes (40.14 mL, 1.6 M) under argon at −78° C. was added diisopropylamine (6.5 g, 64.2 mmol, 1.0 eq). After stirring for 30 min at −78° C., a solution of 2-fluoropyridine (6.23 g, 64.2 mmol, 1.0 eq) in anhydrous THF (50 mL) was added. The reaction mixture was stirred at −78° C. for 4 h. Iodine (16.3 g, 64.2 mmol, 1.0 eq) was then added, and the reaction mixture was stirred at −78° C. for another 30 min. The reaction was hydrolyzed with 10% water-THF, and diluted with ethyl acetate and water. The organic layer was washed with water, brine, and dried. The solvent was evaporated under reduced pressure, and the crude product was purified on a MPLC (Biotage) eluted with 20/8020 v/v ethyl acetate-hexane to give 2-fluoro-3-iodo-pyridine as a yellow oil (8.50 g, 59.4%). ¹H-NMR (Acetone-d₆) [symbol] 8.14 (m, 2H), 6.94 (m, 1H); GC-MS (M⁺=223, R_{f}=9.50 min); R_{f}=0.70 (30% ethyl acetate-hexane).

### Example 15

### Method H-8

### Preparation of 3-iodo-2-methoxy-pyridine

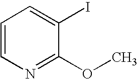

To a solution of sodium methoxide (8.0 mL, 35.9 mmol, 4.0 eq, 25% in methanol) in methanol (60 mL) was added 2-fluoro-3-iodo-pyridine (2.0 g, 8.97 mmol). The reaction mixture was refluxed under argon for 1 h. The reaction mixture was diluted with ethyl acetate and water. The organic layer was washed with water, brine, and dried over sodium sulfate. The solvent was removed at reduced pressure to give 1.8 g (85.4%) of crude product as a yellow oil. ¹H-NMR (Acetone-d₆) [symbol] 8.16 (m, 2H), 6.78 (m, 1H), 3.93 (s, 3H); LC-MS (ES MH⁺=236.2); R_{f}=0.75 (30% ethyl acetate-hexane).

### Example 16

### Method H-9

### Preparation of (3-iodo-pyridin-2-yl)-methylamine

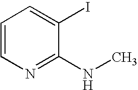

To a solution of 40% methylamine in water (60 mL) was added 2-fluoro-3-iodo-pyridine (2.0 g, 8.97 mmol), and the reaction mixture was refluxed under argon for 4 h. The cooled reaction was diluted with ethyl acetate and water. The organic layer was washed with water, brine, and dried. The solvent was evaporated under reduced pressure to give 1.70 g (81.0%) of crude product. ¹H-NMR (Acetone-d₆) [symbol] 8.06 (dd, J=4.8, 1.5 Hz, 1H), 7.89 (dd, J=7.2, 1.8 Hz, 1H), 6.34 (m, 1H), 5.60 (broad, s, 1H), 2.94 (d, J=4.5 Hz, 3H); R_{f}=0.68 (30% ethyl acetate-hexane).

### Example 17

### Method H-10

### Preparation of cyclopropanecarboxylic acid (3-bromophenyl)amide

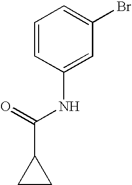

A mixture of 3-bromoaniline (1.0 g, 5.81 mmol), cyclopropane carbonyl chloride (0.61 g, 5.81 mmol, 1.0 eq), and triethylamine (1.17 g, 11.6 mmol, 2.0 eq) in anhydrous THF (20 mL) was stirred at room temperature under argon for 16 h. The reaction mixture was diluted with ethyl acetate and water. The organic layer was washed with water, brine, and dried over magnesium sulfate. The solvent was removed at reduced pressure to afford 1.05 g (75.2%) of the crude product. ¹H-NMR (Acetone-d₆) δ 8.60 (broad s, 1H), 8.07 (dd, J=3.6, 2.1 Hz, 1H), 7.52 (m, 1H), 7.22 (m, 2H), 1.73 (m 1H), 0.90 (m, 2H), 0.80 (m, 2H); MS ES (MH⁺=242); R_{f}=0.46 (30% ethyl acetate-hexane).

### Example 18

### Method H-11

### Preparation of 3-Bromo-N-(2-methoxy-ethyl)-benzenesulfonamide

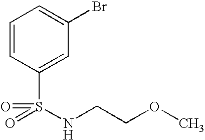

A solution of 3-bromobenzenesulfonyl chloride (1.0 g, 3.72 mmol), 2-methoxyethylamine (0.84 g, 11.15 mmol, 3.0 eq), potassium carbonate (2.57 g, 18.59 mmol, 5.0 eq) in acetone (10.0 mL) was stirred at 40° C. for 4 h. The reaction was diluted with ethyl acetate, washed with water, brine, and dried over magnesium sulfate. The solvent was removed at reduced pressure and purified on the MPLC (Biotage) eluted with 20-25% ethyl acetate-hexane to afford 1.05 g (96%) of the product. R_{f}=0.33 (silica, ethyl acetate:hexanes, 3:7); ¹H-NMR (DMSO-d₆) δ 7.94 to 7.76 (m, 4H), 7.54 (t, J=7.9 Hz, 1H), 3.27 (t, J=5.6 Hz, 2H), 3.13 (s, 3H), 2.93 (q, J=5.6 Hz, 2H).

### Example 19

### Method H-12

### Preparation of diethyl-(3-iodo-benzyl)-amine

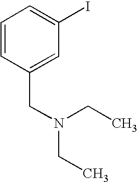

A solution of 3-bromophenacyl bromide (1.0 g, 3.20 mmol), diethylamine (0.70 g, 9.60 mmol, 3.0 eq), potassium carbonate (1.33 g, 9.60 mmol, 3.0 eq) in acetone (10.0 mL) was stirred at 40° C. for 4 h. The reaction was diluted with ethyl acetate, washed with water, brine, and dried over magnesium sulfate. The solvent was removed at reduced pressure and purified on the MPLC (Biotage) eluted with 5-8% ethyl acetate-hexane to afford 0.92 g (99%) of the product. R_{f}=0.28 (silica, ethyl acetate:hexanes, 1:9); ¹H-NMR (DMSO-d₆) δ 7.66 (bs, 1H), 7.59 to 7.55 (m, 1H), 7.33 to 7.29 (m, 1H), 7.10 (t, J=7.8 Hz, 1H), 3.47 (s, 2H), 2.42 (q, J=7.1 Hz, 4H), 0.95 (t, J=6.9, 6H).

### Example 20

### Method H-13

### Preparation of 3-bromo-N-methyl-benzamide

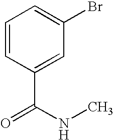

A suspension of methylamine hydrochloride (0.9 g, 13.40 mmol, 3.0 eq) and triethyl amine (2.26 g, 22.33 mmol, 5.0 eq) in anhydrous methylene chloride (10 mL) was cooled to 0° C. The cooled suspension was treated with 3-bromobenzoyl chloride (1.0 g, 4.47 mmol) and then allowed to stir at room temperature for 4 h. The reaction was diluted with ethyl acetate, washed with water, brine, and dried over magnesium sulfate. The solvent was removed at reduced pressure and purified on the MPLC (Biotage) eluted with 35-45% ethyl acetate-hexane to afford 0.60 g (63%) of the product. R_{f}⁼0.28 (silica, ethyl acetate:hexanes, 2:3); ¹H-NMR (DMSO-d₆) δ 8.55 (bs, 1H), 7.99 (t, J=1.7 Hz, 1H), 7.83 to 7.79 (m, 1H), 7.73 to 7.69 (m, 1H), 7.42 (t, J=8.0 Hz, 1H), 2.77 (d, J=6.7 Hz, 3H).

### Example 21

### Method H-14

### Preparation of 2-(3-bromo-phenyl)-acetamide

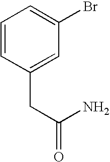

A solution of 3-bromophenylacetonitrile (1.0 g, 5.10 mmol) in acetone (25 mL) and water (15 mL) was treated with sodium percarbonate. The reaction was stirred at 60° C. overnight. The organic solvent was removed at reduced pressure and the residue was diluted with ethyl acetate and water. The layers were separated and the organic was washed with brine and dried over magnesium sulfate. The solvent was removed at reduced pressure and the residue was washed with diethyl ether-hexanes (1/1, v/v) to afford 0.65 g of product (60%) as a white solid. R_{f}=0.18 (silica, ethyl acetate:hexanes, 3:2); ¹H-NMR (DMSO-d₆) δ 7.50 (bs, 1H), 7.46 to 7.39 (m, 2H), 7.26 to 7.22 (m, 2H), 6.93 (bs, 1H), 3.37 (s, 2H).

### Example 22

### Method H-15

### Preparation of 2-(3-Bromo-phenyl)-propan-2-ol

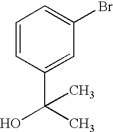

A solution of 3N methylmagnesium bromide (6.53 mL, 19.59 mmol, 3 eq) in diethyl ether was cooled to 0° C. and treated with 3-bromoacetophenone (1.3 g, 6.53 mmol). The reaction was stirred at room temperature for 4 h. The reaction was diluted with ethyl acetate and water. The layers were separated and the organic was washed with saturated sodium bicarbonate, 2N HCl, brine and dried over magnesium sulfate. The solvent was removed at reduced pressure and purified on the MPLC (Biotage) eluted with 5-10% ethyl acetate-hexane to afford 1.2 g (90%) of the product. R_{f}=0.22 (silica, ethyl acetate:hexanes, 1:9); ¹H-NMR (DMSO-d₆) δ 7.63 (t, J=1.8 Hz, 1H), 7.45 to 7.35 (m, 2H), 7.25 (t, J=7.7, 1H), 5.15 (s, 1H), 1.39 (s, 6H).

### General Method B: Preparation of Cyanophenols, Cyanothiophenols and Conversion to Formula (I) Compounds

In these methods, cyanophenols or thiophenols (II) are prepared from readily available phenols or thiophenols, then coupled with (III) to provide the products of formula (I) as shown in the Reaction Scheme for General Method B, and in the specific examples below for this method where X═O

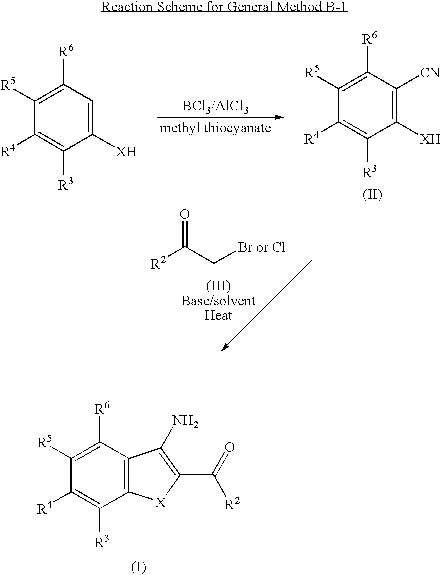

### Example 23

### Method B-1

### Preparation of 3-amino-6-phenyl-1-benzofuran-2-yl)(2,4-dichlorophenyl)methanone

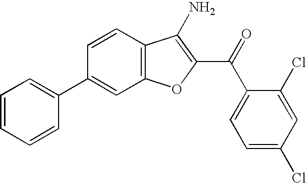

### Step 1: Preparation of starting material 2-cyano-5-phenylphenol

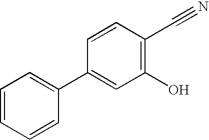

To a stirred solution of 3-phenylphenol (10.0 g, 58.75 mmol) in anhydrous tetrahydrofuran (50 mL) and anhydrous dichloroethane (50 mL) was added, at 0° C., 1.0 M boron trichloride in dichloromethane (64.6 mL, 64.6 mmol, 1.1 eq) followed by methyl thiocyanate (4.4 mL, 64.6 mmol, 1.1 eq) and aluminum chloride (7.83 g, 58.75 mmol, 1.0 eq). The reaction mixture was stirred at room temperature for 2 days and then cooled to 0° C. To the dark brown reaction mixture was added 50% aqueous sodium hydroxide solution (150 mL) until pH reached above 10. The resulting yellow bi-phasic layers were stirred at reflux for 1 h and then cooled to room temperature. The bi-phasic layers were separated, and the aqueous layer was adjusted to pH 3 with 2.0 N hydrogen chloride solution (˜300 mL) at 0° C. The acidified aqueous mixture was extracted with ethyl acetate (3×400 mL), and the combined organic layers were dried (MgSO₄), filtered, and concentrated under reduced pressure. Crystallization from ether-hexane (150 mL) gave 2-cyano-5-phenylphenol as a white solid (5.81 g, 47.2%). ¹H-NMR (DMSO-d₆) δ 11.20 (s, 1H), 7.66 (d, J=8.7 Hz, 1H), 7.62 to 7.59 (m, 2H), 7.51 to 7.42 (m, 3H), 7.22 to 7.19 (m, 2H), R_{f}=0.08, 25% ethyl acetate-hexane.

### Step 2: Preparation of the title compound 3-amino-6-phenyl-1-benzofuran-2-yl)(2,4-dichlorophenyl)methanone

To a stirred solution of 2-cyano-5-phenylphenol from step 1 (5.71 g, 29.25 mmol) and 2-chloro-1-(2,4-dichlorophenyl)ethanone (7.19 g, 32.17 mmol, 1.1 eq) in anhydrous N,N-dimethylformamide (50 mL) was added potassium carbonate (4.85 g, 35.1 mmol, 1.2 eq), and the orange reaction mixture was stirred at 90° C. for 17 h. The resulting dark wine color reaction was poured into ethyl acetate (500 mL) and water (300 mL). The ethyl acetate layer was washed with saturated aqueous ammonium chloride, water, and brine. The organic layer was then dried (MgSO₄), filtered, and evaporated in vacuo. The crude product was purified on silica get (flash column chromatography) eluted with 10% ethyl acetate-hexane followed by 20% ethyl acetate-hexane. Crystallization from ether-hexane afforded the benzofuran product as a yellow solid (7.56 g, 67.6%). ¹H-NMR (DMSO-d₆) δ 8.10 (d, J=8.4 Hz, 1H), 7.75 (d, J=3.6 Hz, 1H), 7.74 (d, J=3.0 Hz, 1H), 7.71 (m, 2H), 7.62 to 7.53 (m, 5H), 7.47 to 7.35 (m, 3H); MS LC-MS (MH⁺=382); Anal. calculated for C₂₁H₁₃Cl₂NO₂: 65.99% H, 3.43% N, 3.66%, found C, 65.70% H, 3.40% N, 3.72%; melting point (uncorrected) 144 to 146.5° C.

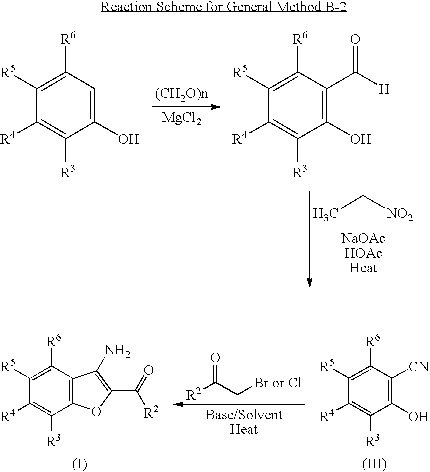

### Example 24

### Method B-2a

### Preparation of [3-Amino-6-(2-methyl-oxazol-4-yl)-benzofuran-2-yl]-(2-methoxy-phenyl)-methanone

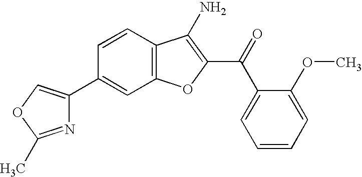

### Step 1: Preparation of intermediate 4-(3-methoxy-phenyl)-2-methyl-oxazole

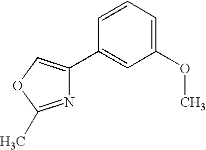

To the solution of 2-bromo-3′-methoxy-acetophenone (1.9 g, 8.1 mmol) in 15 mL toluene was added acetamide (1.2 g, 20.3 mmol, 2.5 eq). The reaction was stirred at 110° C. for 40 h. Filtered off the white solid and washed with ethyl acetate. Evaporated the filtrate (added some methanol to lower the boiling point) and the washings in vacuo. Purification using MPLC (Biotage) gave 1.1 g (72%) of 4-(3-methoxy-phenyl)-2-methyl-oxazole as a light yellow liquid. ¹H-NMR(CH₃OH-d₄) δ 8.11 (s, 1H), 7.25 to 7.28 (m, 3H), 6.83 to 6.86 (m, 1H), 3.82 (s, 3H), 2.49 (s, 3H); R_{f}=0.36, 25% ethyl acetate-hexane.

### Step 2: Preparation of intermediate 3-(2-Methyl-oxazol-4-yl)-phenol

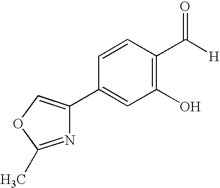

To a solution of 4-(3-methoxy-phenyl)-2-methyl-oxazole (1.1 g, 5.8 mmol) from step 1 in anhydrous DCM (5 mL) was added 1M boron tribromide in DCM (18 mL, 17.4 mmol, 3 eq) in an ice bath. The reaction mixture was stirred at room temperature for 2 h. The mixture was poured into ice and ethyl acetate. To this was added about 50 mL 1 N NaOH, followed by saturated aqueous sodium bicarbonate until pH was 8. Separate the organic layer and extracted the aqueous layer with EtOAc twice. Combined the organic layers and evaporated in vacuo. 0.88 g (87%) 3-(2-Methyl-oxazol-4-yl)-phenol was obtained as a yellow solid. ¹H-NMR(CH₃OH-d₄) δ 8.07 (s, 1H), 7.22 to 7.15 (m, 3H), 6.78 to 6.75 (m, 1H), 2.52 (s, 3H); MS LC-MS (MH⁺=176.3); TLC R_{f}=0.15, 25% EtOAc-HEX.

### Step 3: Preparation of intermediate 2-Hydroxy-4-(2-methyl-oxazol-4-yl)-benzaldehyde

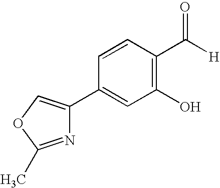

To a solution of 3-(2-Methyl-oxazol-4-yl)-phenol (0.88 g, 5.0 mmol) from step 2 in anhydrous acetonitrile (20 mL) was added magnesium chloride (1.4 g, 15 mmol, 3 eq), triethylamine (2.8 mL, 20 mmol, 4 eq) and paraformaldehyde (0.6 g, 20 mmol, 4 eq). The reaction mixture was refluxed for 17 h. The starting material was completely gone. Added some water and saturated aqueous ammonium chloride until pH=7. At this point some red solid precipitated. Filtered off the red solid and extracted the filtrate with EtOAc 3 times. Most of the red solid was dissolved in MeOH. Combined the EtOAc extract and MeOH filtrate and dried over magnesium sulfate. It was evaporated in vacuo and gave 2-Hydroxy-4-(2-methyl-oxazol-4-yl)-benzaldehyde 1.0 g (98%) as a yellow solid. ¹H-NMR(CH₃OH-d₄) δ 10.0 (s, 1H), 8.3 (s, 1H), 7.73 (d, J=8 Hz 1H), 7.4 (dd, J=8 Hz, 1.6 Hz, 1H), 7.34 (d, J=1.6 Hz, 1H), 2.54 (s, 3H); TLC R_{f}=0.24, 25% EtOAc-HEX.

### Step 4: Preparation of intermediate 2-Hydroxy-4-(2-methyl-oxazol-4-yl)-benzonitrile

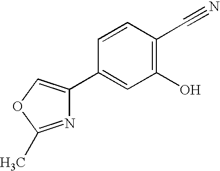

To a solution of 2-hydroxy-4-(2-methyl-oxazol-4-yl)-benzaldehyde (1 g, 4.9 mmol) from step 3 in acetic acid (5 mL) was added nitroethane (0.74 g, 9.8 mmol, 2 eq) and sodium acetate (0.8 g, 9.8 mmol, 2 eq). The reaction mixture was refluxed for 17 h. The starting material was completely gone. Added some water and neutralized the solution with saturated aqueous sodium bicarbonate until pH=7. Extracted with EtOAc 3 times. Combined the extracts and evaporated in vacuo. Purification using MPLC (Biotage) gave 0.2 g (20%) 2-Hydroxy-4-(2-methyl-oxazol-4-yl)-benzonitrile as a light yellow solid. ¹H-NMR(CH₃OH-d₄) δ 8.2 (s, 1H), 7.51 (d, J=8 Hz, 1H), 7.31 (d, J=1.6 Hz, 1H), 7.26 (dd, J=8 Hz, 1.6 Hz, 1H), 2.51 (s, 3H).

### Step 5: Preparation of [3-Amino-6-(2-methyl-oxazol-4-yl)-benzofuran-2-yl]-(2-methoxy-phenyl)-methanone

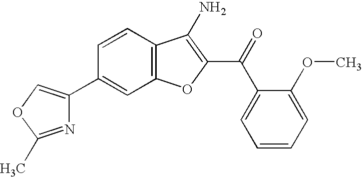

To a solution of 2-hydroxy-4-(2-methyl-oxazol-4-yl)-benzonitrile from step 4 (25 mg, 0.12 mmol) and 2-methoxyphenacyl bromide (31 mg, 0.14 mmol, 1.1 eq) in anhydrous N,N-dimethylformamide (2 mL) was added potassium carbonate (34 mg, 0.25 mmol, 2 eq). The reaction mixture was shaken at 90° C. for 17 h. The mixture was cooled to room temperature and poured into ethyl acetate and water. The aqueous layer was extracted with ethyl acetate twice. Combined the organic layers and evaporated in vacuo. Purification using HPLC gave 19 mg (43%) of [3-Amino-6-(2-methyl-oxazol-4-yl)-benzofuran-2-yl]-(2-methoxy-phenyl)-methanone as a yellow solid. ¹H-NMR(CH₃OH-d₄) δ 8.23 (s, 1H), 7.86 (d, J=8 Hz, 1H), 7.65 to 7.39 (m, 4H), 7.13 (d, J=8 Hz, 1H), 7.05 (t, J=7.2 Hz, 1H), 3.82 (s, 3H), 2.51 (s, 3H). MS LC-MS (MH⁺=349.2); R_{f}=0.33, 50% EtOAc-HEX.

### Example 25

### Method B-2b

### Preparation of [3-Amino-6-(2-methyl-thiazol-4-yl)-benzofuran-2-yl]-(2,4-dichloro-phenyl)-methanone

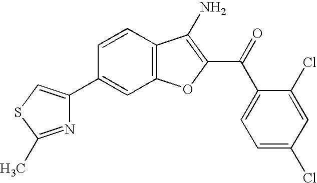

### Step 1: preparation of intermediate 4-(3-methoxy-phenyl)-2-methyl-thiazole

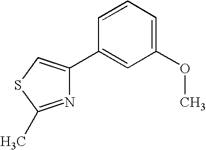

To the solution of 2-bromo-3′-methoxy-acetophenone (1.0 g, 4.4 mmol, 1.2 eq) in 10 mL anhydrous ethanol was added thioacetamide (0.27 g, 3.6 mmol, 1 eq). Some solid was formed immediately. The reaction was stirred at 80° C. for 1 h. The reaction was cooled to rt and then was placed in an ice bath for a while. The white solid was filtered, washed with hexane and dried in vacuum oven to give 0.67 g (90%) of 4-(3-methoxy-phenyl)-2-methyl-thiazole. ¹H-NMR(CH₃OH-d₄ and a little DMSO-d₆) δ 7.53 (s, 1H), 7.39 (m, 1H), 7.29 to 7.26 (m, 1H), 7.18 (m, 1H), 6.78 (m, 1H), 3.74 (s, 3H), 2.91 (s, 3H); MS LC-MS MH⁺=206.3; R_{f}=0.13, 2% EtOAc-HEX.

### Step 2: Preparation of intermediate 3-(2-Methyl-thiazol-4-yl)-phenol

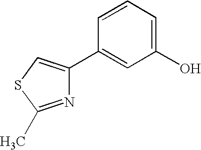

The same procedure was used as in the preparation of 3-(2-methyl-oxazol-4-yl)-phenol method described in Example 24 above. Yield 74%. ¹H-NMR(CH₃OH-d₄) δ 7.54 (s, 1H), 7.32 to 7.19 (m, 3H), 6.77 to 6.74 (m, 1H), 2.76 (s, 3H); TLC R_{f}=0.57, 50% EtOAc-HEX.

### Step 3: Preparation of intermediate 2-Hydroxy-4-(2-methyl-thiazol-4-yl)-benzaldehyde

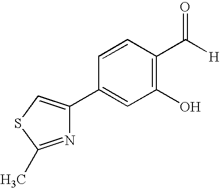

The procedure used was the same as that described in the preparation of 2-hydroxy-4-(2-methyl-oxazol-4-yl)-benzaldehyde. TLC R_{f}=0.71, 50% EtOAc-HEX. The crude product was used in step 4 without further purification.

### Step 4: Preparation of intermediate 2-Hydroxy-4-(2-methyl-thiazol-4-yl)-benzonitrile for use in making

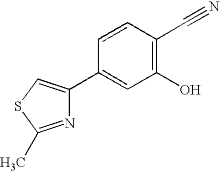

The same procedure was used as described in the preparation of the intermediate 2-hydroxy-4-(2-methyl-oxazol-4-yl)-benzonitrile. Two steps overall yield was 83%. ¹H-NMR(CH₃OH-d₄) δ 7.75 (s, 1H), 7.52 (d, J=8.2 Hz, 1H), 7.48 (d, J=1.6 Hz, 1H), 7.42 (dd, J=8.2 Hz, 1.6 Hz, 1H), 2.75 (s, 3H). MS LC-MS MH⁺=217.2; R_{f}=0.18, 30% EtOAc-HEX.

### Step 5: Preparation of 2 [3-Amino-6-(2-methyl-thiazol-4-yl)-benzofuran-2-yl]-(2,4-dichloro-phenyl)-methanone

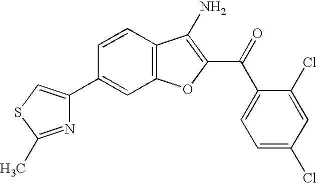

The same procedure was used as described in the preparation of Example 24, step 5. Yield 33%. ¹H-NMR (DMSO-d₆) δ 8.09 (s, 1H), 8.06 (dd, J=8.2 Hz, 0.8 Hz, 1H), 7.94 (m, 1H), 7.89 (dd, J=8.2 Hz, 1.2 Hz, 1H), 7.75 (dd, J=2 Hz, 0.4 Hz, 1H), 7.59 (dd, 8.2 Hz, 0.4 Hz, 1H), 7.55 (dd, J=8.2 Hz, 1.6 Hz, 1H), 2.71 (s, 3H). MS LC-MS (MH⁺=403.2/405.2); R_{f}=0.57, 50% EtOAc-HEX.

### Example 26

### Method B-2c

### Preparation of [3-Amino-6-(1-methyl-1H-pyrazol-3-yl)-benzofuran-2-yl]-(2,4-dichloro-phenyl)-methanone

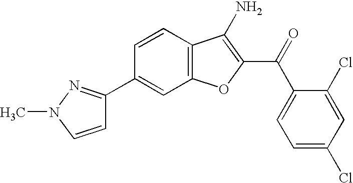

### Step 1: Preparation of intermediate 3-(1-methyl-1H-pyrazol-3-yl)-phenol

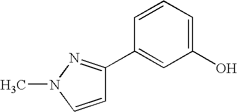

3-hydroxy-acetophenone (1 g, 7.3 mmol) and N,N-dimethylformamide-dimethyl acetal (2.6 g, 22 mmol, 3 eq) were shaken in a 40 mL vial at rt for 17 h. The mixture was evaporated in vacuo and obtained both phenol product and methyl phenol ether. To the solution of this mixture in 10 mL anhydrous ethanol was added methyl hydrazine (1 g, 22 mmol, 3 eq). The reaction mixture was shaken at 80° C. for 2 h. The mixture was evaporated in vacuo. Boron tribromide (3 eq) in dichloromethane was used to de-methylate the methyl ether as described in the preparation of Example 24 step 2. Some methyl ether still existed and the mixture was used for step 2 without further purification.

### Step 2: Preparation of intermediate 2-Hydroxy-4-(1-methyl-1H-pyrazol-3-yl)-benzonitrile

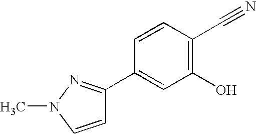

The same procedure was used as described in the preparation of Example 24, step 3 and step 4: 250 mg of 2-Hydroxy-4-(1-methyl-1H-pyrazol-3-yl)-benzonitrile and its isomer 2-Hydroxy-6-(1-methyl-1H-pyrazol-3-yl)-benzonitrile were obtained as a yellow solid. The mixture was used in step 3 without further purification. MS LC-MS MH⁺=200.1; TLC R_{f}=0.16, 50% EtOAc-HEX.

### Step 3: Preparation of [3-Amino-6-(1-methyl-1H-pyrazol-3-yl)-benzofuran-2-yl]-(2,4-dichloro-phenyl)-methanone

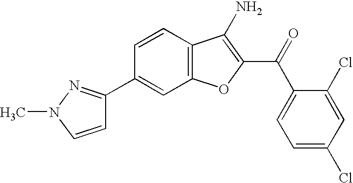

The same procedure was used as described for the preparation of Example 24 step 5. [3-Amino-6-(1-methyl-1H-pyrazol-3-yl)-benzofuran-2-yl]-(2,4-dichloro-phenyl)-methanone was obtained as a light yellow solid. Yield was 12%. ¹H-NMR (CDCl₃) δ 7.7 to 7.8 (m, 2H), 7.60 (d, J=10.8 Hz, 1H), 7.51 to 7.48 (m, 2H), 7.38 to 7.33 (m, 2H), 6.55 (d, J=3.2 Hz, 1H), 5.99 (broad, s, 2H); 3.94 (s, 3H). MS LC-MS MH⁺=386.2/388.2; TLC R_{f}=0.3, 50% EtOAc-HEX.

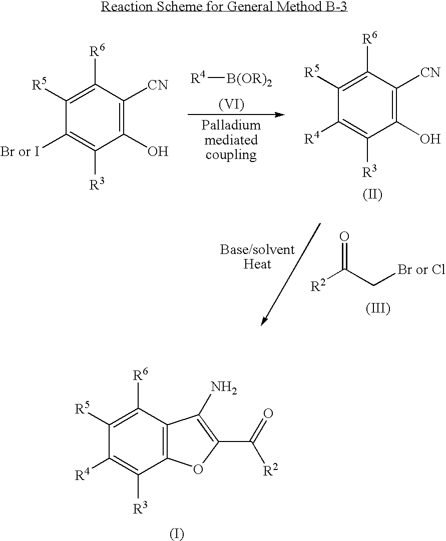

### Example 27

### Method B-3

### Preparation of (3-Amino-6-pyridin-3-yl-benzofuran-2-yl)-(2-methoxy-phenyl)-methanone

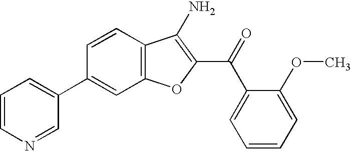

### Step 1: Preparation of starting material: 2-Benzyloxy-4-pyridin-3-yl-benzonitrile

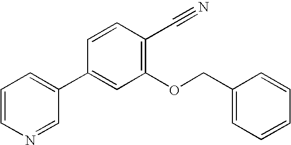

This compound was prepared from 2-benzyloxy-4-iodo-benzonitrile (2.0 g, 5.97 mmol) in the manner described for [3-amino-6-(pyridin-3-yl)-1-benzofuran-2-yl](2,4-dichlorophenyl)methanone, affording 1.42 g (83%) of a tan solid. ¹H-NMR (DMSO-d₆) δ 8.98 (d, J=1.8 Hz, 1H), 8.64 (dd, J=5.1 Hz, 1.5 Hz, 1H), 8.18 (dt, J=8.0 Hz, 2.1 Hz, 1H), 8.86 (d, J=7.8 Hz, 1H), 7.68 (d, J=1.2 Hz, 1H), 7.56 to 7.32 (m, 7H), 5.42 (s, 2H); LC-MS (ES MH⁺=287, RT=2.39 min); R_{f}=0.08 (25% ethyl acetate-hexane).

### Step 2: Preparation of starting material: 2-hydroxy-4-(pyridin-3-yl)benzonitrile

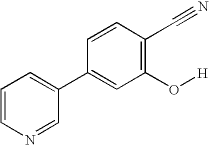

To a dry flask charged with 10% Pd/C (160.0 mg, 0.56 mmol, 0.2 eq) was added a solution of 2-benzyloxy-4-pyridin-3-yl-benzonitrile (800.0 mg, 2.79 mmol) in 1:1 v/v ethyl acetate-ethanol (28.0 mL). The reaction mixture was hydrogenated under an atmosphere of hydrogen supplied by an attached balloon for 16 h. The reaction was filtered through a pad of celite, and the filtrate was concentrated to give 536.4 mg (97.8%) of a white solid. ¹H-NMR (DMSO-d₆) δ 11.21 (broad s, 1H), 8.82 (dd, J=2.4 Hz, 0.6 Hz, 1H), 8.61, (dd, J=5.1 Hz, 1.8 Hz, 1H), 8.02 (ddd, J=7.8 Hz, 2.1 Hz, 1.2 Hz, 1H), 7.71 (d, J=8.4 Hz, 1H), 7.50 (ddd, J=8.1 Hz, 4.5 Hz, 0.6 Hz, 1H), 7.27 to 7.22 (m, 2H); LC-MS (ES MH⁺=197, RT=0.97 min); R_{f}=0.16 (75% ethyl acetate-hexane).

### Step 3: Preparation of the title compound: (3-Amino-6-pyridin-3-yl-benzofuran-2-yl)-(2-methoxy-phenyl)-methanone

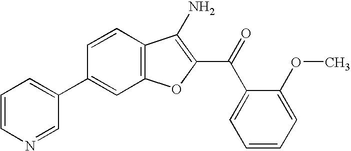

To a stirred solution of 2-hydroxy-4-(pyridin-3-yl)benzonitrile (60.0 mg, 0.31 mmol) and 2-bromo-2′-methoxyacetophenone (70.1 mg, 0.31 mmol, 1.0 eq) in anhydrous N,N-dimethylformamide (5.0 mL) was added potassium carbonate (84.5 mg, 0.62 mmol, 2.0 eq), and the orange reaction mixture was stirred at 80° C. for 17 h. The resulting dark wine color reaction was poured into ethyl acetate (100 mL) and water (50 mL). The ethyl acetate layer was washed with saturated aqueous ammonium chloride, water, and brine. The organic layer was then dried over sodium sulfate, filtered, and evaporated under reduced pressure. The crude product was purified on the MPLC (Biotage) eluted with 50% ethyl acetate-hexane. Crystallization from ether-hexane afforded the benzofuran as a yellow solid (24.4 mg, 23.2%). ¹H-NMR (Acetone-d₆) δ 8.98 (d, J=1.5 Hz, 1H), 8.60 (dd, J=7.2, 1.5 Hz, 1H), 8.12 (m, 2H), 7.70 (d, J=1.5 Hz, 1H), 7.65 (dd, J=6.3, 0.9 Hz, 1H), 7.52 to 7.41 (m, 3H), 7.17 (d, J=7.5 Hz, 1H), 7.06 (t, J=6.6 Hz, 1H), 6.84 (broad, s, 2H), 2.85 (s, 3H); LC-MS (ES MH⁺=345, RT=1.97 min).

### Example 28

### Method B-4

### Preparation of N-{3-[3-amino-2-(2,4-dichloro-benzoyl)-benzofuran-6-yl]-pyridin-1-yl}-propionamide

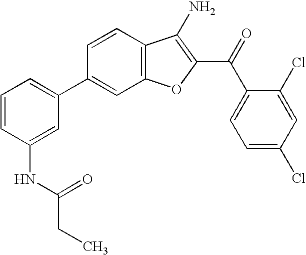

### Step 1: Preparation of 3′-amino-3-benzyloxy-biphenyl-4-carbonitrile

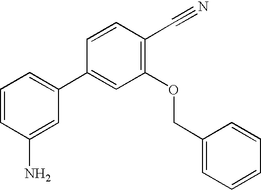

A solution of 2-(benzyloxy)-4-iodobenzonitrile (6.20 g, 18.5 mmol) in 1,2-dimethoxyethane was degassed with argon for 30 min. At this time, tetrakis(triphenyl phosphine)palladium(0), (2.13 g, 1.85 mmol, 0.1 eq) was added followed by 3-aminophenyl boronic acid (2.53 mg, 18.5 mmol, 1.0 eq) and 2M aqueous Na₂CO₃ (4.0 mL). The reaction was bubbled with argon for another 10 min and then heated to 80° C. overnight (18 h). The reaction was diluted with ethyl acetate, washed with water, brine, and dried over magnesium sulfate. The solvent was removed at reduced pressure and purified on the MPLC (Biotage) eluted with 30% ethyl acetate-hexane to afford 3.33 g (59.9%) of a yellow solid as the product. ¹H-NMR (Acetone) [symbol] 07.71 (d, J=8.1 Hz, 1H), 7.58 (m, 2H), 7.48 to 7.30 (m, 5H), 7.18 (m, 1H), 6.99 (m, 1H), 6.90 (m, 1H), 6.76 (m, 1H), 5.44 (s, 2H), 4.81 (broad, s, 2H); R_{f}=0.32 (30% ethyl acetate-hexane).

### Step 2: Preparation of N-(3′-benzyloxy-4′-cyano-biphenyl-3-yl)-N-propionyl-propionamide

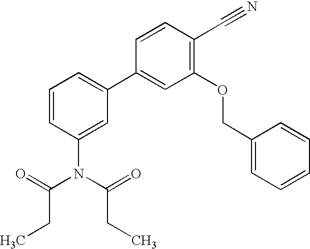

To a solution of 3′-amino-3-benzyloxy-biphenyl-4-carbonitrile (800 mg, 2.66 mmol) in dichloromethane (100 mL) at 0° C. was added dropwise propionyl chloride (370 mg, 4.00 mmol, 1.5 eq) followed by triethylamine (405 mg, 4.00 mmol, 1.5 eq). The reaction was stirred at 0° C. under argon for 1 h. The reaction was concentrated, and the residue was dissolved in ethyl acetate. The organic layer was washed with water, brine, and dried over magnesium sulfate. The solvent was removed at reduced pressure and purified on the MPLC (Biotage) eluted with 30% ethyl acetate-hexane to afford 980 mg (89.2%) of a yellow solid as the product. ¹H-NMR (Acetone-d₆) [symbol] 7.76 (m, 2H), 7.69 (m, 1H), 7.64 to 7.55 (m, 4H), 7.48 to 7.36 (m, 5H), 5.45 (s, 2H), 2.61 (q, J=6.9 Hz, 4H), 1.05 (t, J=6.6 Hz, 6H); R_{f}=0.42 (30% ethyl acetate-hexane).

### Step 3: Preparation of N-(4′-cyano-3′-hydroxy-biphenyl-3-yl)-N-propionyl-propionamide

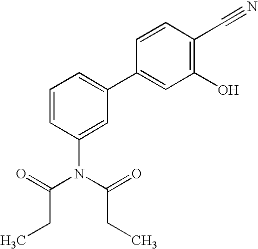

To a dry flask charged with 10% Pd/C (124.0 mg, 0.13 eq) was added a solution of N-(3′-benzyloxy-4′-cyano-biphenyl-3-yl)-N-propionyl-propionamide (980 mg, 2.38 mmol) in 1:1 v/v ethyl acetate-ethanol (10 mL). The reaction mixture was hydrogenated under an atmosphere of hydrogen supplied by an attached balloon for 24 h. The reaction was filtered through a pad of celite, and the filtrate was concentrated. Purification on the MPLC (Biotage) eluted with 30% ethyl acetate-hexane afforded 332 mg (43.4%) of the product. ¹H-NMR (Acetone-d₆) [symbol] 9.95 (broad s, 1H), 7.75 to 7.58 (m, 4H), 7.35 (m, 3H), 2.61 (q, J=7.2 Hz, 4H), 1.05 (t, J=7.2 Hz, 6H); LC-MS (ES MH⁺=323) R_{f}=0.20 (30% ethyl acetate-hexane).

### Step 4: Preparation of the title compounds: N-{3-[3-amino-2-(2,4-dichloro-benzoyl)-benzofuran-6-yl]-phenyl}-N-propionyl-propionamide

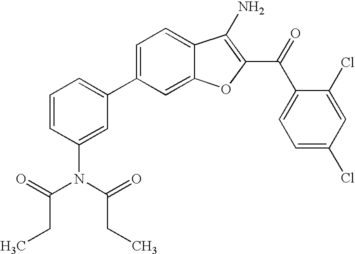

To N-(4′-cyano-3′-hydroxy-biphenyl-3-yl)-N-propionyl-propionamide (70.0 mg, 0.22 mmol) and 2,2′,4′-trichloro-acetophenone (48.5 mg, 0.22 mmol, 1.0 eq) in anhydrous N,N-dimethylformamide (5 mL) was added potassium carbonate (60.0 mg, 0.43 mmol, 2.0 eq). The reaction mixture was stirred under argon at 80° C. for 16 h. The brown reaction mixture was cooled and diluted with ethyl acetate and water. The organic layer was washed with saturated aqueous ammonium chloride, water, brine, and dried over sodium sulfate. The solvent was evaporated under reduced pressure, and the crude product was purified on the MPLC (Biotage) eluted with 20% ethyl acetate-hexane to give 39.6 mg (35.8%) of the product. ¹H-NMR (Acetone-d₆) [symbol] 8.10 (d, J=8.4 Hz, 1H), 7.83 (d, J=6.3 Hz, 1H), 7.72 to 7.53 (m, 7H), 7.30 (m, 1H), 7.08 (broad, s, 2H), 2.63 (q, J=7.2 Hz, 4H), 1.04 (t, J=7.2 Hz, 6H); MS ES (MH⁺=509); R_{f}=0.25 (30% ethyl acetate-hexane).

### Step 5: Preparation of N-{3-[3-amino-2-(2,4-dichloro-benzoyl)-benzofuran-6-yl]-pyridin-1-yl}-propionamide

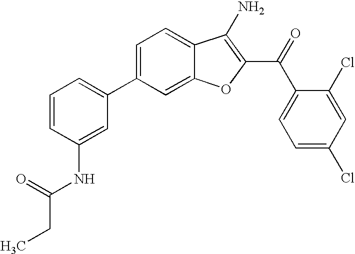

To N-{3-[3-amino-2-(2,4-dichloro-benzoyl)-benzofuran-6-yl]-phenyl}-N-propionyl-propionamide (230 mg, 0.45 mmol) in anhydrous THF (5 mL) was added 2 N aq. NaOH (0.46 mL, 0.90 mmol, 2.0 eq). The reaction mixture was stirred at reflux for 16 h. The reaction mixture was diluted with ethyl acetate and water. The organic layer was washed with saturated aqueous ammonium chloride, water, brine, and dried over sodium sulfate. The solvent was evaporated under reduced pressure and the crude product was purified on the MPLC (Biotage) eluted with 20% ethyl acetate-hexane to give 161 mg (78.4%) of a yellow solid. ¹H-NMR (Acetone-d₆) δ 9.02 (broad s, 1H), 8.00 (m, 1H), 7.95 (d, J=6.9 Hz, 1H), 7.53 to 7.40 (m, 6H), 7.28 (m, 2H), 6.96 (broad s, 2H), 2.26 (q, J=7.8 Hz, 2H), 1.04 (t, J=4.5 Hz, 3H); MS ES (MH⁺=453); R_{f}=0.28 (30% ethyl acetate-hexane).

### Example 29

### Method B-5

### Exemplified by the [3-amino-6(4-methyl-thiophen-3-yl)-benzofuran-2-yl]-(2,4-dichloro-phenyl)-methanone

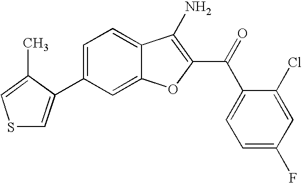

### Step 1: Preparation of 2-Hydroxy-4-(4-methyl-thiophen-3-yl)-benzonitrile

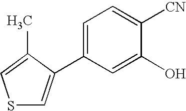

4-Bromo-2-methoxy-benzonitrile (2.4 g, 11.32 mmol) was dissolved into DMF (25 mL). To this solution was added bis(pinacolato)diboron (3 g, 11.88 mmol), palladium(II)acetate (0.76 g, 0.34 mmol), and potassium acetate (3.3 g, 34 mmol). This mixture was degassed by purging with Ar for 15 min and heated to 80° C. for 5 h. To the mixture was then added 3-bromo-4-methyl-thiophene (1.8 g, 10.2 mmol), cesium carbonate (5.53 g, 17 mmol), and tetrakis(triphenylphosphine)palladium(0). The solution was stirred for 18 h at 80° C. The reaction mixture was poured into an ethyl acetate:water (1:1, 200:200 mL) system. The organic was separated and further product was extracted using EtOAc (3×200 mL). The organic layers were combined, washed with brine (100 mL) and dried using sodium sulfate. The organic layer was concentrated in vacuo and the crude product was dissolved into methylene chloride (2.5 mL) and cooled to 0° C. Aluminum chloride (0.726 g, 5.45 mmol) was added and the solution was stirred for 5 min. Ethane thiol (0.339 g, 5.45 mmol) was then added and the solution was stirred for 2 h at it. Water (10 mL) was added to quench the reaction and the product was extracted from the aqueous layer via methylene chloride (3×20 mL). The organics were combined and dried with sodium sulfate. The organic solution was then concentrated in vacuo. Flash chromatography (10% EtOAc:HEX→30% EtOAc:Hex) yielded 0.231 g (9.75%) of 2-hydroxy-4-(4-methyl-thiophen-3-yl)-benzonitrile. ¹HNMR (methylene chloride d₂) [symbol] 7.56 (d, J=8.5 Hz, 1H), 7.31 (d, J=4.2 Hz, 1H), 7.25-7.03 (m, 4H), 2.29 (s, 3H). LC-MS RT: 3.34 min, [M+H]+: 216.1.

### Step 2: Preparation of [3-amino-6(4-methyl-thiophen-3-yl)-benzofuran-2-yl]-(2,4-dichloro-phenyl)-methanone

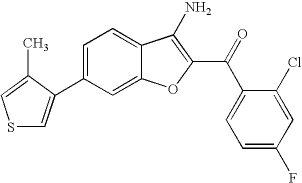

To a stirred solution of 2-hydroxy-4-(4-methyl-thiophen-3-yl)-benzonitrile from step 1 (0.050 g, 0.23 mmol) and 2-bromo-1-(2-chloro-4-fluoro-phenyl)-ethanone (0.071 g, 0.28 mmol, 1.2 eq) in anhydrous N,N-dimethylformamide (2 mL) was added potassium carbonate (0.048 g, 0.35 mmol, 1.5 eq), and the orange reaction mixture was stirred at 80° C. for 18 h. The resulting dark, wine-colored reaction was poured into ethyl acetate (5 mL) and water (5 mL). The ethyl acetate layer was washed with water and brine. The organic layer was then dried (MgSO₄), filtered, and evaporated in vacuo. The crude product was taken up in acetonitrile (2 mL) and purified by HPLC (10% acetonitrile:water→90% acetonitrile:water). The benzofuran product was collected as a yellow solid (0.066 g, 40.0%). ¹H-NMR (CD₂Cl₂) δ 7.72 (d, J=7.6 Hz, 1H), 7.63-7.584 (m, 1H), 7.38-7.30 (m, 4H), 7.18-7.09 (m, 2H), 6.05 (s, 2H), 2.30 (s, 3H); MS LC-MS (MH⁺=386.2), RT=4.12 min.

Additional compounds illustrated in Table 1 were prepared as described above by choosing the appropriate starting materials that are readily available and/or the synthesis of which is taught herein, and using the processes of Methods A and/or B described above or other standard chemical processes known in the art.

TABLE 1
Examples Synthesized using Method B

| | | R_{f} (TLC solvent) | LC/MS | Synthesis | Synthesis |
|---|---|---|---|---|---|
| Example | Structure | Or RT (min)* | ([M + H]+) | of (III)** | of (I) |
| 30 | 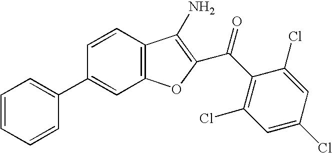 | R_{f} = 0.41 [25% EtOAc/HEX] | 416.0 | A-1 | B-1 |
| 31 | 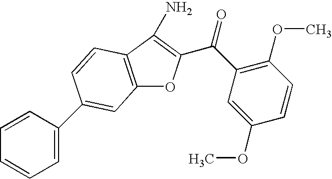 | R_{f} = 0.50 [30% EtOAc/HEX] | 374.0 | comm | B-1 |
| 32 | 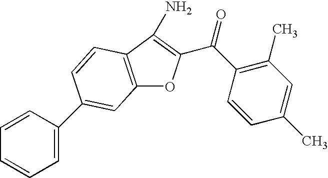 | R_{f} = 0.45 [30% EtOAc/HEX] | 342.0 | comm | B-1 |
| 33 | 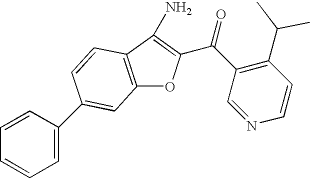 | R_{f} = 0.30 [30% EtOAc/HEX] | 357.0 | A-4 | B-1 |
| 34 | 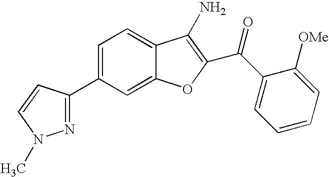 | R_{f} = 0.14 @N 50/50 EtOAc/HEX | 348.2 | comm | B-2 |
| 35 | 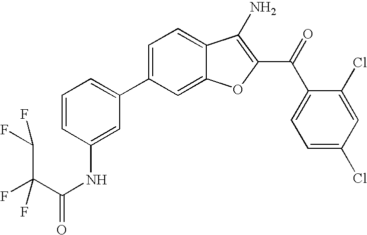 | R_{f} = 0.43, HEX/EtOAc = 70/30 | 525.0 | comm | B-4 |
| 36 | 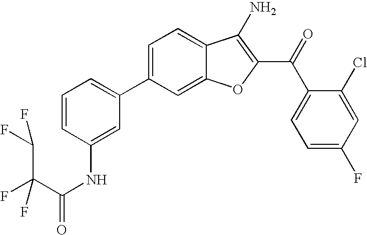 | R_{f} = 0.42, HEX/EtOAc = 70/30 | 509.0 | comm | B-4 |
| 37 | 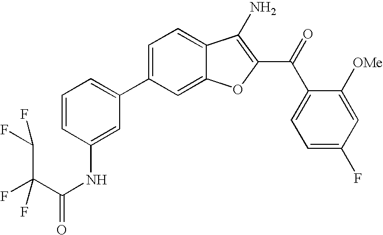 | R_{f} = 0.28, HEX/EtOAc = 70/30 | 505.0 | comm | B-4 |
| 38 | 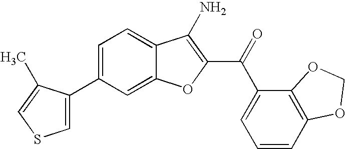 | RT = 3.91 | 378.2 | A-5 | B-5 |
| 39 | 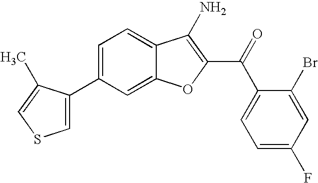 | RT = 4.09 | 430/432 | A-2 | B-5 |
| 40 | 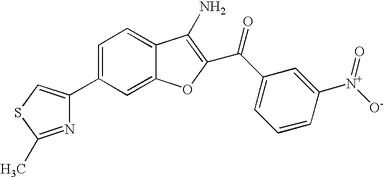 | R_{f} = 0.51 [50% EtOAc/HEX] | 380 | comm | B-2 |
| 41 | 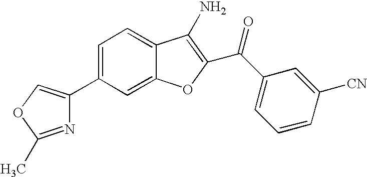 | R_{f} = 0.4 [50% EtOAc/HEX] | 344 | comm | B-2 |
| 42 | 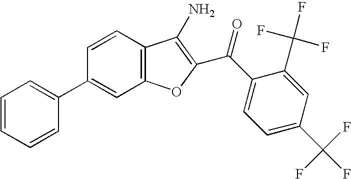 | R_{f} = 0.34 [25% EtOAc/Hex] | 450 | A-2 | B-1 |
| 43 | 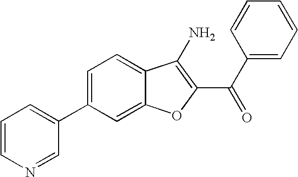 | RT = 2.81 | 315.0 | comm | B-3 |

Footnotes:
*The following are the LCMS conditions: HPLC-electrospray mass spectra (HPLC ES-MS) were obtained using a Gilson HPLC system equipped with two Gilson 306 pumps, a Gilson 215 Autosampler, a Gilson diode array detector, a YMC Pro C-18 column (2 × 23 mm, 120 A), and a Micromass LCZ single quadrupole mass spectrometer with z-spray electrospray ionization. Spectra were scanned from 120-1000 amu over 2 seconds. ELSD (Evaporative Light Scattering Detector) data was also acquired as an analog channel. Gradient elution was used with Buffer A as 2% acetonitrile in water with 0.02% TFA and Buffer B as 2% water in Acetonitrile with 0.02% TFA at 1.5 mL/min. Samples were eluted as follows: 90% A for 0.5 min ramped to 95% B over 3.5 min and held at 95% B for 0.5 min and then the column is brought back to initial conditions over 0.1 min. Total run time is 4.8 min.
**comm means commercially available.

General Method C: Preparation of Formula (I) compounds via 6-iodo-benzofurans (IV)

Illustrated in the Reaction Scheme for General Method C below is a generally applicable method for the preparation of compounds of formula (I) via intermediates of formulas (IV) and (V). The condensation of properly substituted 2-cyano-5-iodo-phenol (II) and 1-aryl-2-haloethanone (III) under basic conditions (such as cesium carbonate, potassium carbonate, sodium carbonate, DBU), in a solvent such as DMF, MeCN at temperatures between room temperature to 100° C. to give 6-iodo-benzofuran (IV). Palladium mediated coupling reactions between (IV) and arylboronic acids or boronates (VI) afford the desired compounds. Alternatively, 6-iodo-benzofuran (IV) was converted to boronate (V), which was then used to prepare the desired compounds via palladium mediated coupling with arylhalides (VII).

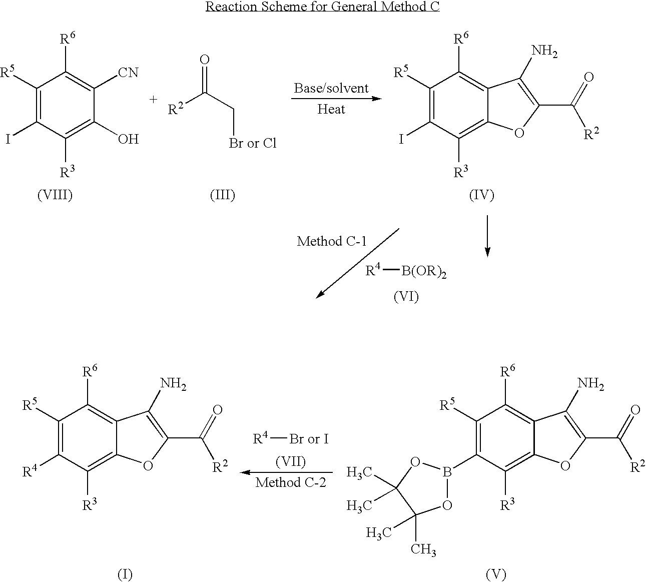

### Example 44

### Method C-1a

### Preparation of [3-amino-6-(3-pyridinyl)-1-benzofuran-2-yl](2,4-dichlorophenyl)methanone

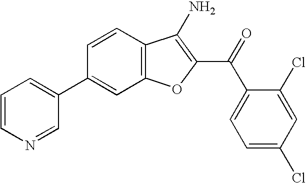

### Step 1: Preparation of the starting material: 2-Cyano-5-iodophenol

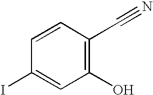

To a stirred solution of 3-iodophenol (20.0 g, 90.9 mmol) in anhydrous dichloroethane (60 mL) was added, at 0° C., 1.0 M boron trichloride in dichloromethane (100 mL, 100.0 mmol, 1.1 eq), followed by methyl thiocyanate (6.85 mL, 100.0 mmol, 1.1 eq) and aluminum chloride (12.1 g, 90.9 mmol, 1.0 eq). The reaction mixture was stirred at room temperature for 3 days and then cooled to 0° C. To the dark brown reaction mixture was added 50% aqueous sodium hydroxide solution (150 mL) until pH 11. The resulting yellow biphasic layers were stirred at reflux for 3 h and then cooled to room temperature. The biphasic layers were separated, and the aqueous layer was adjusted to pH 1 with 50% aqueous hydrogen chloride solution at 0° C. The acidified aqueous mixture was extracted with ethyl acetate (3×400 mL), and the combined organic layers were dried (Na₂SO₄), filtered, and concentrated under reduced pressure. Crystallization from ether-hexane (200 mL) gave 2-cyano-5-iodophenol as a white solid (14.8 g, 66.4%). ¹H-NMR (DMSO-d₆) δ 11.43 (s, 1H), 7.38 to 7.36 (m, 2H), 7.29 (dd, J=8.4, 1.5 Hz, 1H); MS GC-MS (M⁺=245; RT=7.45 min); R_{f}=0.16, 25% ethyl acetate-hexane.

### Step 2: Preparation of the intermediate: (3-amino-6-iodo-1-benzofuran-2-yl)(2,4-dichlorophenyl)methanone

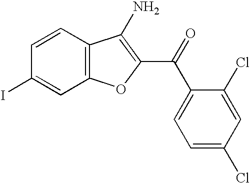

To a stirred solution of 2-cyano-5-iodophenol (3.68 g, 15.0 mmol) and 2,2′,4′-trichloroacetophenone (4.02 g, 18.0 mmol, 1.2 eq) in anhydrous N,N-dimethylformamide (15 mL) was added potassium carbonate (3.11 g, 22.5 mmol, 1.5 eq), and the orange reaction mixture was stirred at 80° C. for 16 h. The resulting dark wine color reaction was poured into ethyl acetate (500 mL) and water (300 mL). The ethyl acetate layer was washed with saturated aqueous ammonium chloride, water, and brine. The organic layer was then dried (Na₂SO₄), filtered, and evaporated under reduced pressure. The crude product was purified on the MPLC (Biotage) eluted with 20% ethyl acetate-hexane. Crystallization from dichloromethane-hexane afforded the benzofuran as a yellow solid (6.16 g, 95.0%). ¹H-NMR (DMSO-d₆) δ 7.88 (d, J=1.2 Hz, 1H), 7.82 (d, J=8.4 Hz, 1H), 7.74 (dd, J=1.5, 0.9 Hz, 1H), 7.61 (dd, J=8.4, 1.5 Hz, 1H), 7.55 to 7.51 (m, 4H); LC-MS (ES MH⁺=432/434).

### Step 3: Preparation of the title compound: [3-amino-6-(3-pyridinyl)-1-benzofuran-2-yl](2,4-dichloro-phenyl)methanone

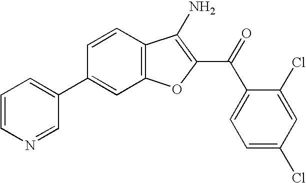

A solution of (3-amino-6-iodo-1-benzofuran-2-yl)(2,4-dichlorophenyl)methanone (2.0 g, 4.63 mmol) in toluene (10 mL) and ethanol (10 mL) was degassed with argon for 10 min. At this time, pyridine-3-boronic acid (740 mg, 6.02 mmol, 1.3 eq) was added followed by [1,1′-bis(diphenylphosphino)-ferrocene]dichloro-palladium(II), complex with dichloromethane (1:1) (378 mg, 0.46 mmol, 0.1 eq) and 2M aqueous Na₂CO₃ (11.6 mL). The reaction was bubbled with argon for another 10 min and then heated to 80° C. overnight. The reaction was diluted with ethyl acetate, washed with water, brine, and dried over magnesium sulfate. The solvent was removed at reduced pressure and purified on the MPLC (Biotage) eluted with 45 to 65% ethyl acetate-hexane to afford 1.69 g (95.3%) of a yellow solid as the product. ¹H-NMR (DMSO-d₆) δ 8.95 (d, J=2.4 Hz, 1H), 8.56 (dd, J=4.5, 1.5 Hz, 1H), 8.14 (d, J=8.4 Hz, 2H), 7.83 (s, 1H), 7.76 (d, J=1.8 Hz, 1H), 7.66 (dd, J=8.1, 1.2 Hz, 1H), 7.58 to 7.53 (m, 4H), 7.47 (dd, J=8.4, 4.8 Hz, 1H); LC-MS (ES MH⁺=383/385, RT=2.58 min). Anal. calculated for C₂₀H₁₂Cl₂N₂O₂: C 62.68% H 3.16% N, 7.31%, found C, 62.41% H, 3.18% N, 7.23%.

### Example 45a

### Method C-1b

### Preparation of (3-Amino-5-fluoro-6-pyridin-3-yl-benzofuran-2-yl)-(2,4-dichloro-phenyl)-methanone

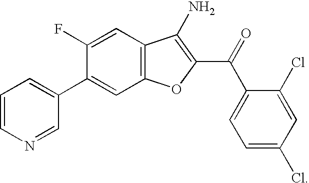

### Step 1: Preparation of starting material: 4-amino-2-benzyloxy-5-fluorobenzonitrile

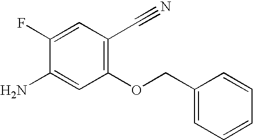

A mixture of 4-amino-2,5-difluorobenzonitrile (500 mg, 3.24 mmol), benzyl alcohol (385.9 mg, 3.57 mmol, 1.1 eq), potassium carbonate (896.2 mg, 6.49 mmol, 2.0 eq), and 4 angstroms molecular sieves (500 mg) in anhydrous N,N-dimethylformamide (6.5 mL) was stirred at 100° C. under argon for 24 h. The reaction was diluted with ethyl acetate, washed with water, brine, and dried over sodium sulfate. The solvent was removed at reduced pressure, and the crude material was purified on the MPLC (Biotage) eluted with 15% ethyl acetate-hexane to afford 155.0 mg (19.7%) of the product. ¹H-NMR (DMSO-d₆) δ 7.42 to 7.32 (m, 6H), 6.49 (d, J=7.5 Hz, 1H), 6.27 (broad s, 2H), 5.09 (s, 2H); LC-MS (ES MH⁺=243, RT=2.75 min); R_{f}=0.27 (25% ethyl acetate-hexane).

### Step 2: Preparation of starting material: 5-fluoro-2-hydroxy-4-iodobenzonitrile

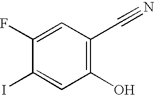

To a slurry of 4-amino-2-benzyloxy-5-fluorobenzonitrile (155.0 mg, 0.64 mmol) in concentrated aq. HCl (2.6 mL) at 0° C. was added sodium nitrite (66.2 mg, 0.96 mmol, 1.5 eq) dissolved in water (1.0 mL). After stirring at 0° C. for 1 h, a solution of potassium iodide (159.3 mg, 0.96 mmol, 1.5 eq) dissolved in water (5.1 mL) was added, and the reaction mixture was stirred at ambient temperature for 3 days. The reaction was diluted with ethyl acetate, washed with water, brine, and dried over sodium sulfate. The solvent was removed at reduced pressure, and the crude material was purified on the MPLC (Biotage) eluted with 25% ethyl acetate-hexane to afford 63.0 mg (37.4%) of the product. ¹H-NMR (DMSO-d₆) δ 10.20 (broad s, 1H), 7.54 (d, J=5.1 Hz, 1H), 7.47 (d, J=7.2 Hz, 1H); R_{f}=0.16 (25% ethyl acetate-hexane).

### Step 3: Preparation of starting material: 3-amino-6-iodo-1-benzofuran-2-yl)(2,4-dichlorophenyl) methanone

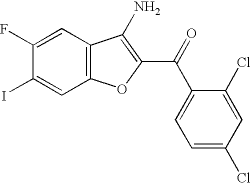

To a stirred solution of 5-fluoro-2-hydroxy-4-iodobenzonitrile (60 mg, 0.23 mmol) and 2, 2′,4′-trichloroacetophenone (76.5 mg, 0.34 mmol, 1.5 eq) in anhydrous N,N-dimethylformamide (3.3 mL) was added potassium carbonate (47.3 mg, 0.34 mmol, 1.5 eq), and the orange reaction mixture was stirred at 80° C. for 16 h. The resulting dark wine color reaction was poured into ethyl acetate (100 mL) and water (50 mL). The ethyl acetate layer was washed with saturated aqueous ammonium chloride, water, and brine. The organic layer was then dried over sodium sulfate, filtered, and evaporated under reduced pressure. The crude product was purified on the MPLC (Biotage) eluted with 15% ethyl acetate-hexane to afford 41.0 mg (39.9%) of the product. ¹H-NMR (Acetone-d₆) δ 7.95 (d, J=4.5 Hz, 1H), 7.83 (d, J=7.5 Hz, 1H), 7.65 (d, J=2.1 Hz, 1H), 7.61 (s, 1H), 7.56 (d, J=1.8 Hz, 1H), 7.00 (broad s, 2H); LC-MS (ES MH⁺=450, RT=3.78 min); R_{f}=0.39 (25% ethyl acetate-hexane).

### Step 4: Preparation of the title compound: (3-Amino-5-fluoro-6-pyridin-3-yl-benzofuran-2-yl)-(2,4-dichloro-phenyl)-methanone

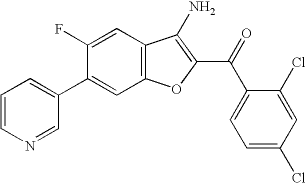

This compound was prepared from (3-amino-6-iodo-1-benzofuran-2-yl)(2,4-dichlorophenyl)methanone_(38.0 mg, 0.08 mmol) in the manner described for [3-amino-6-(pyridin-3-yl)-1-benzofuran-2-yl](2,4-dichlorophenyl)methanone, affording 13.0 mg (38.4%) of the product. ¹H-NMR (Acetone-d₆) δ 8.84 (t, J=1.8 Hz, 1H), 8.64 (dd, J=4.8 Hz, 1.5 Hz, 1H), 8.06 to 8.01 (m, 1H), 7.92 (d, J=10.2 Hz, 1H), 7.66 (d, J=1.8 Hz, 1H), 7.63 (s, 1H), 7.62 (d, J=6.0 Hz, 1H), 7.56 (dd, J=8.1 Hz, 2.1 Hz, 1H), 7.45 (ddd, J=7.2 Hz, 4.8 Hz, 0.9 Hz, 1H), 7.05 (broad s, 2H); LC-MS (ES MH⁺=401, RT=2.66 min).

There may be slight variations in the above step 3 procedure with respect to the palladium catalyst used in the palladium-mediated coupling reactions, as illustrated by the following example:

### Example 45b

### Preparation of [3-Amino-6-(2-methyl-phenyl)-benzofuran-2-yl]-(2,4-dichloro-phenyl)-methanone

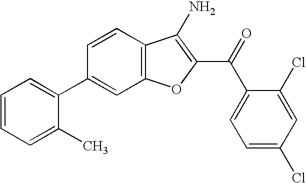

In a 7 mL vial with stirring was placed 75 mg (0.17 mmol, 1 eq) of (3-amino-6-iodo-1-benzofuran-2-yl)(2,4-dichlorophenyl)methanone in 2.5 mL of argon degassed DME. To this was added 10 mg (0.01 mmol, 0.05 eq) of Pd(PPh₃)₄ and the vial allowed to shake for 5 min. At this point, 29.1 mg (0.21 mmol, 1.2 eq) of 2-methylphenyl boronic acid and 0.43 mL (0.43 mmol, 2.5 eq) of 1 M Na₂CO₃ were added and the reaction allowed to shake at 80° C. overnight under argon. The volatiles were then removed and the residue purified via Prep TLC (25% EtOAc/Hex) to provide 42.9 mg (62%) of the desired product as a yellow solid. ¹H-NMR (DMSO-d₆) δ 7.91 (d, 1H), 7.63 (d, 1H), 7.56 (d, 1H), 7.49 (dd, 1H), 7.34-7.24 (m, 8H), 2.28 (s, 3H), LC-MS (+esi MH⁺=396.3, RT=3.86 min), TLC R_{f}=0.48 (25% EtOAc/Hex).

### Example 46

### Method C-2

### Preparation of [3-amino-6-(2-methyl-3-pyridinyl)-1-benzofuran-2-yl](2,4-di-chlorophenyl)methanone

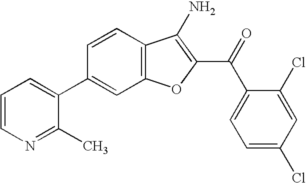

### Step 1: Preparation of starting material: [3-amino-6-(4,4,5,5-tetramethyl-1,3,2-dioxaborolan-2-yl)-1-benzofuran-2-yl](2,4-dichlorophenyl)methanone

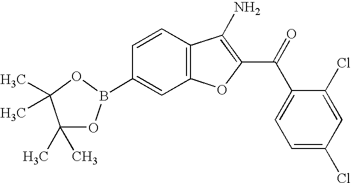

A solution of (3-amino-6-iodo-1-benzofuran-2-yl)(2,4-dichlorophenyl)methanone (225 mg, 0.52 mmol) in 1,4-dioxane (2.6 mL) was degassed with argon for 30 min. At this time, [1,1′-bis(diphenylphosphino)-ferrocene]dichloropalladium (II), complex with dichloromethane (1:1) (12.8 mg, 0.02 mmol, 0.03 eq) was added followed by triethylamine (0.22 mL, 1.56 mmol, 3.0 eq) and pinacolborane (0.13 mL, 0.89 mmol, 1.7 eq). The reaction was bubbled with argon for another 10 min and then heated to 80° C. overnight. The reaction was diluted with ethyl acetate, washed with water, brine, and dried over sodium sulfate. The solvent was removed at reduced pressure and purified on the MPLC (Biotage) eluted with 15% ethyl acetate-hexane to afford 154.5 mg (68.7%) of an orange foam. ¹H-NMR (DMSO-d₆) δ 8.05 (d, J=7.8 Hz, 1H), 7.76 (d, J=2.4 Hz, 1H), 7.58 to 7.48 (m, 6H), 1.28 (s, 12H); LC-MS (ES MH⁺=432/434, RT=3.97 min).

### Step 2: Preparation of the tile compound: [3-amino-6-(2-methyl-3-pyridinyl)-1-benzofuran-2-yl](2,4-dichlorophenyl)methanone

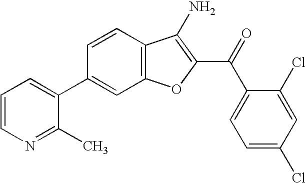

A solution of [3-amino-6-(4,4,5,5-tetramethyl-1,3,2-dioxaborolan-2-yl)-1-benzo-furan-2-yl](2,4-dichlorophenyl)methanone (65 mg, 0.15 mmol) in toluene (1.0 mL) and ethanol (1.0 mL) was degassed with argon for 30 min. At this time, 3-bromo-2-methylpyridine (33.6 mg, 0.20 mmol, 1.3 eq) was added followed by [1,1′-bis(diphenylphosphino)-ferrocene]dichloro-palladium(II), complex with dichloromethane (1:1) (12.3 mg, 0.02 mmol, 0.1 eq) and 2M aqueous Na₂CO₃ (0.38 mL). The reaction was bubbled with argon for another 15 min and then heated to 80° C. overnight. The reaction was diluted with ethyl acetate, washed with water, brine, and dried over magnesium sulfate. The solvent was removed at reduced pressure and purified on the MPLC (Biotage) eluted with 45 to 65% ethyl acetate-hexane to afford 23 mg (38.5%) of a yellow solid as the product. ¹H-NMR (DMSO-d₆) δ 8.46 (dd, J=4.5, 1.5 Hz, 1H), 8.11 (d, J=8.4 Hz, 1H), 7.75 (d, J=2.1 Hz, 1H), 7.64 to 7.56 (m, 5H), 7.46 (s, 1H), 7.31 to 7.27 (m, 2H), 2.41 (s, 3H); LC-MS (ES MH⁺=397/399, RT=2.34 min).

Additional compounds, illustrated in Table 2 below, were prepared in like manner as described above by choosing the appropriate starting materials that are readily available and/or the synthesis of which is taught herein, and using the process described above or other standard chemical processes known in the art.

TABLE 2
Examples of Formula (I) Compounds Synthesized using General Method C

| | | | | | | |
|---|---|---|---|---|---|---|
| | | | | | Synthesis | |
| | | R_{f} (TLC solvent) | LC/MS | Synthesis | of (VI) or | Synth. |
| Example | Structure | Or RT (min)* | ([M + H]+) | of (III)** | (VII)** | of (I) |

| | | | | | | |
|---|---|---|---|---|---|---|
| 47 | 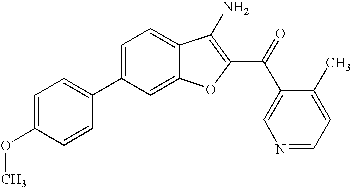 | R_{f} = 0.213 [50% EtOAc/HEX] | 359.0 | A-3 | comm | C-1 |
| 48 | 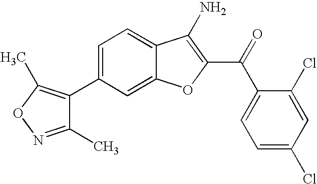 | R_{f} = 0.14 [25% EtOAc/HEX] | 401/403 | comm | comm | C-1 |
| 49 | 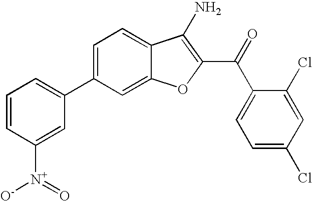 | R_{f} = 0.14 [25% EtOAc/HEX] | 427/429 | comm | comm | C-1 |
| 50 | 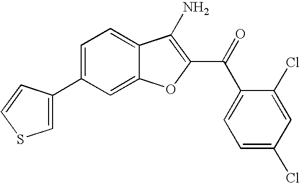 | R_{f} = 0.35 (25% EtOAc/HEX) | 388/390 | comm | comm | C-1 |
| 51 | 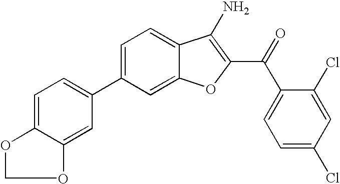 | R_{f} = 0.245 (25% EtOAc/HEX) | 426.0 | comm | comm | C-1 |
| 52 | 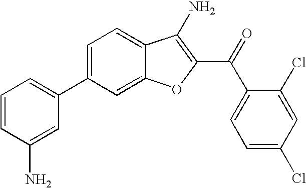 | R_{f} = 0.34 (50% EtOAc/HEX) | 397.0 | comm | comm | C-1 |
| 53 | 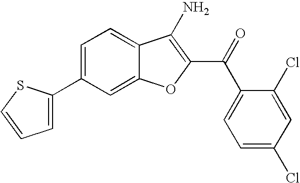 | R_{f} = 0.25 (25% EtOAc/HEX) | 388/390 | comm | comm | C-1 |
| 54 | 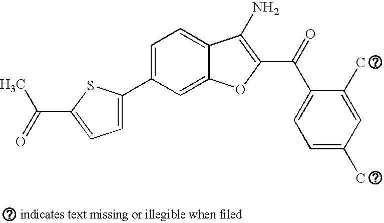 | R_{f} = 0.13 (25% EtOAc/HEX) | 430/432 | comm | comm | C-1 |
| 55 | 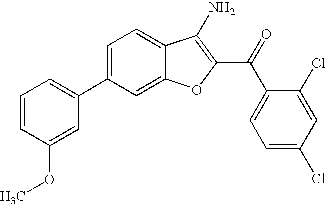 | R_{f} = 0.30 (25% EtOAc/HEX) | 412/414 | comm | comm | C-1 |
| 56 | 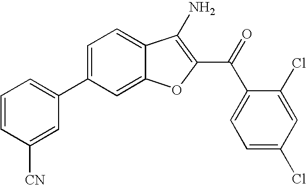 | R_{f} = 0.08 (25% EtOAc/HEX) | 407/409 | comm | comm | C-1 |
| 57 | 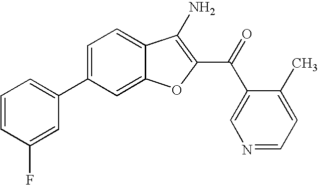 | R_{f} = 0.10, HEX/EtOAc = 50/50 | 347.0 | A-3 | comm | C-1 |
| 58 | 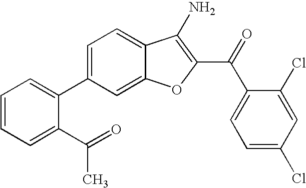 | R_{f} = 0.21 (25% EtOAc/HEX) RT = 3.45 | 424.3 | comm | comm | C-1 |
| 59 | 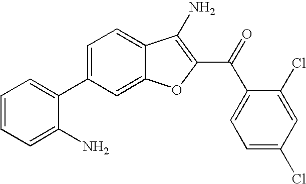 | R_{f} = 0.15 (25% EtOAc/HEX) RT = 3.25 | 397.3 | comm | comm | C-1 |
| 60 | 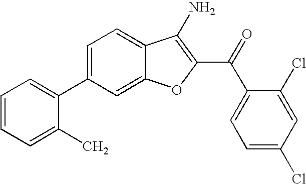 | R_{f} = 0.48 (25% EtOAc/HEX) RT = 3.86 | 396.3 | comm | comm | C-1 |
| 61 | 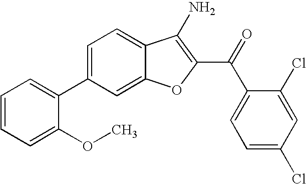 | R_{f} = 0.35 (25% EtOAc/HEX) RT = 3.69 | 412.3 | comm | | C-1 |
| 62 | 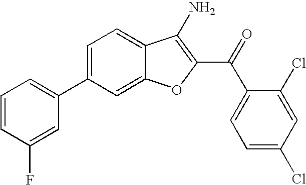 | R_{f} = 0.75 [50% EtOAc/HEX] | 400/402 | comm | comm | C-1 |
| 63 | 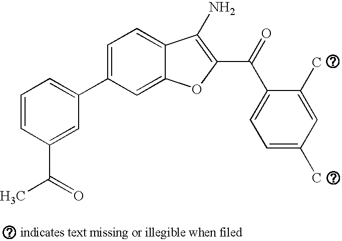 | R_{f} = 0.65 [50% EtOAc/HEX] | 424/426 | comm | comm | C-1 |
| 64 | 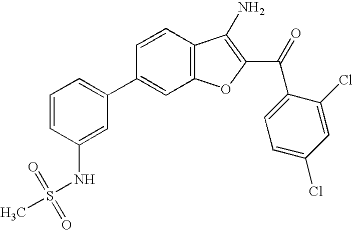 | R_{f} = 0.35 [50% EtOAc/HEX] | 475/477 | comm | comm | C-1 |
| 65 | 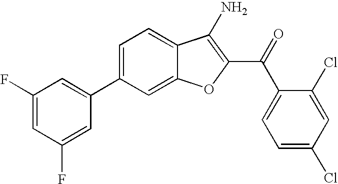 | R_{f} = 0.75 [50% EtOAc/HEX] | 418/420 | comm | comm | C-1 |
| 66 | 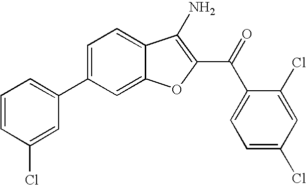 | R_{f} = 0.75 [50% EtOAc/HEX] | 416/418 | comm | comm | C-1 |
| 67 | 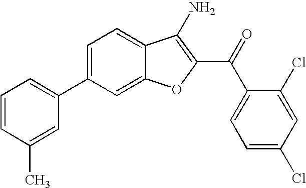 | R_{f} = 0.75 [50% EtOAc/HEX] | 396/398 | comm | comm | C-1 |
| 68 | 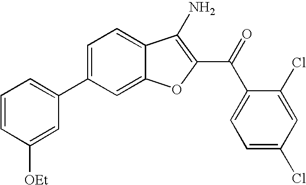 | R_{f} = 0.75 [50% EtOAc/HEX] | 426/428 | comm | comm | C-1 |
| 69 | 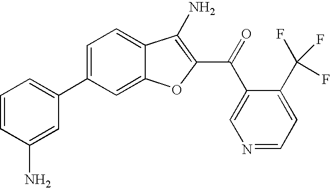 | R_{f} = 0.27 (50% EtOAc/HEX) RT = 2.13 | 398.0 | A-4 | comm | C-1 |
| 70 | 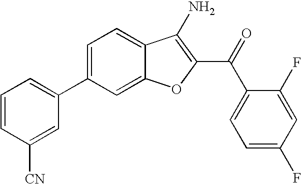 | R_{f} = 0.33 [30% EtOAc/HEX] | 375.0 | comm | comm | C-1 |
| 71 | 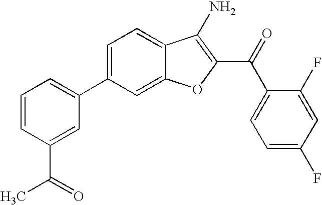 | R_{f} = 0.25, HEX/EtOAc = 70/30 | 392.0 | comm | comm | C-1 |
| 72 | 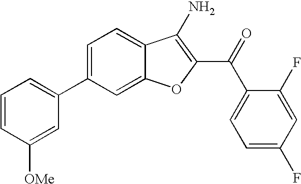 | R_{f} = 0.33, HEX/EtOAc = 70/30 | 380.0 | comm | comm | C-1 |
| 73 | 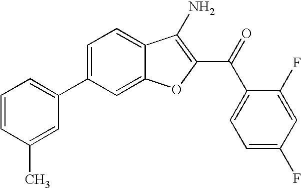 | R_{f} = 0.40 [30% EtOAc/HEX] | 364.0 | comm | comm | C-1 |
| 74 | 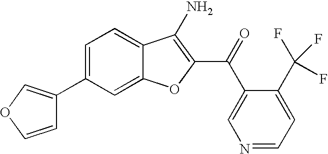 | R_{f} = 0.46 (50% EtOAc/HEX) RT = 3.12 | 373.4 | A-4 | comm | C-1 |
| 75 | 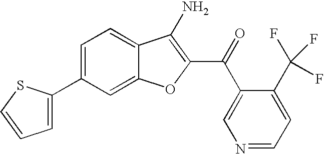 | R_{f} = 0.51 (50% EtOAc/HEX) RT = 3.25 | 389.4 | A-4 | comm | C-1 |
| 76 | 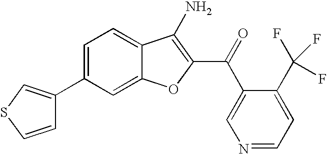 | R_{f} = 0.46 (50% EtOAc/HEX) RT = 3.26 | 389.4 | A-4 | comm | C-1 |
| 77 | 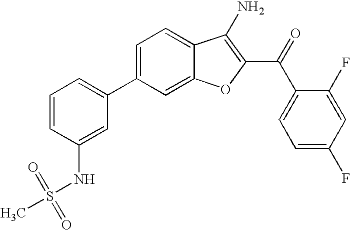 | R_{f} = 0.15, HEX/EtOAc = 50/50 | 443.0 | comm | comm | C-1 |
| 78 | 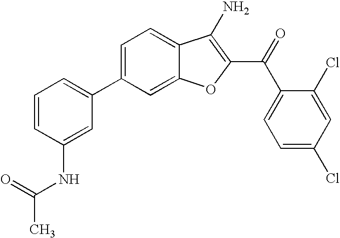 | R_{f} = 0.14 [50% EtOAc/HEX] | 439/441 | comm | comm | C-1 |
| 79 | 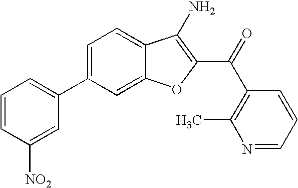 | R_{f} = 0.43 (75% EtOAc/HEX) RT = 2.26 | 374.1 | A-4 | comm | C-1 |
| 80 | 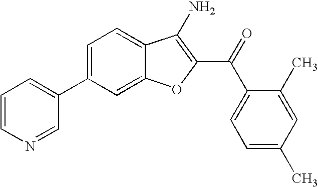 | R_{f} = 0.10 [30% EtOAc/HEX] | 343.0 | comm | comm | C-1 |
| 81 | 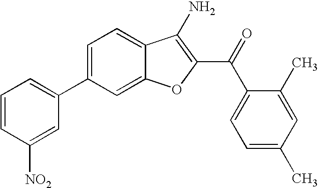 | R_{f} = 0.45 [30% EtOAc/HEX] | 387.0 | comm | comm | C-1 |
| 82 | 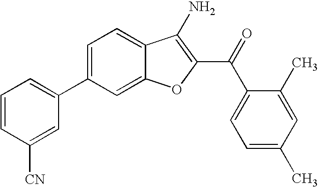 | R_{f} = 0.43 [30% EtOAc/HEX] | 367.0 | comm | comm | C-1 |
| 83 | 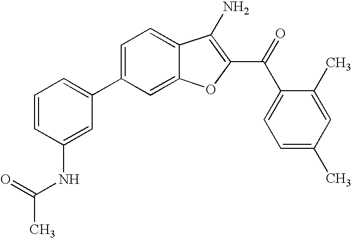 | R_{f} = 0.10 [30% EtOAc/HEX] | 399.0 | comm | comm | C-1 |
| 84 | 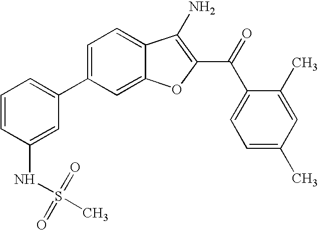 | R_{f} = 0.15 [30% EtOAc/HEX] | 435.0 | comm | comm | C-1 |
| 85 | 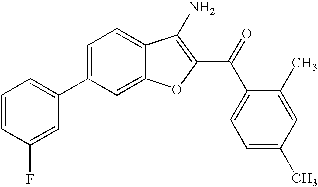 | R_{f} = 0.50 [30% EtOAc/HEX] | 360.0 | comm | comm | C-1 |
| 86 | 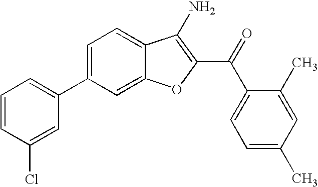 | R_{f} = 0.50 [30% EtOAc/HEX] | 376.0 | comm | comm | C-1 |
| 87 | 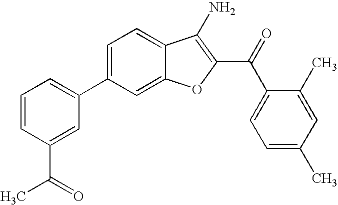 | R_{f} = 0.35 [30% EtOAc/HEX] | 384.0 | comm | comm | C-1 |
| 88 | 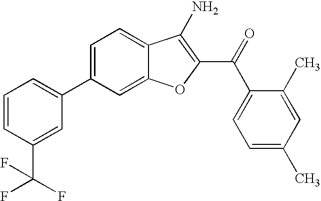 | R_{f} = 0.46 [30% EtOAc/HEX] | 410.0 | comm | comm | C-1 |
| 89 | 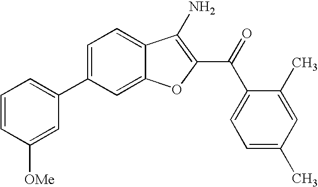 | R_{f} = 0.40 [30% EtOAc/HEX] | 372.0 | comm | comm | C-1 |
| 90 | 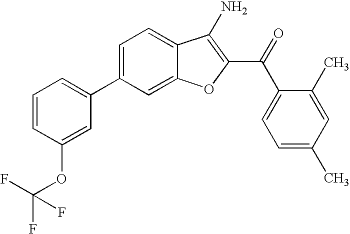 | R_{f} = 0.45 [30% EtOAc/HEX] | 426.0 | comm | comm | C-1 |
| 91 | 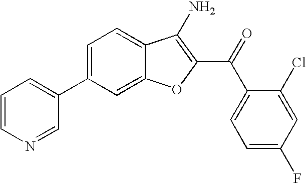 | R_{f} = 0.05, HEX/EtOAc = 70/30 | 367.0 | A-4 | comm | C-1 |
| 92 | 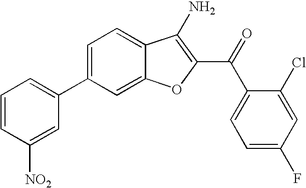 | R_{f} = 0.28, HEX/ET)AC = 70/30 | 411.0 | A-4 | comm | C-1 |
| 93 | 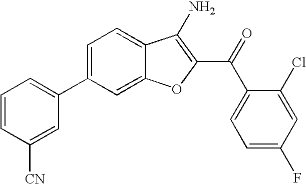 | R_{f} = 0.28, HEX/EtOAc = 70/30 | 391.0 | A-4 | comm | C-1 |
| 94 | 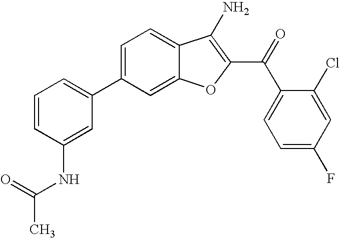 | R_{f} = 0.10, HEX/EtOAc = 70/30 | 423.0 | A-4 | comm | C-1 |
| 95 | 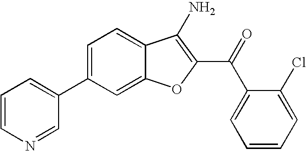 | R_{f} = 0.08, HEX/EtOAc = 70/30 | 349.0 | A-2 | comm | C-1 |
| 96 | 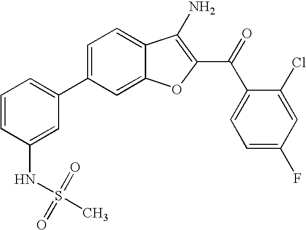 | R_{f} = 0.05, HEX/EtOAc = 70/30 | 459.0 | A-4 | comm | C-1 |
| 97 | 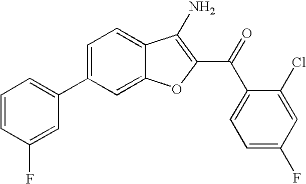 | R_{f} = 0.30, HEX/EtOAc = 70/30 | 384.0 | A-4 | comm | C-1 |
| 98 | 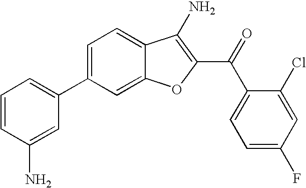 | R_{f} = 0.08, HEX/EtOAc = 70/30 | 381.0 | A-4 | comm | C-1 |
| 99 | 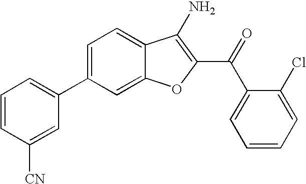 | R_{f} = 0.30, HEX/EtOAc = 70/30 | 373.0 | A-2 | comm | C-1 |
| 100 | 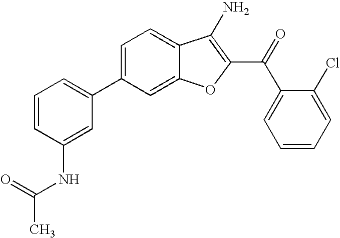 | R_{f} = 0.05, HEX/EtOAc = 70/30 | 405.0 | A-2 | comm | C-1 |
| 101 | 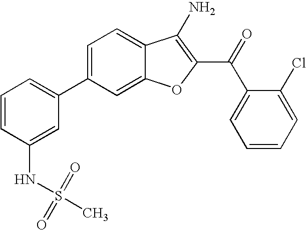 | R_{f} = 0.05, HEX/EtOAc = 70/30 | 441.0 | A-2 | comm | C-1 |
| 102 | 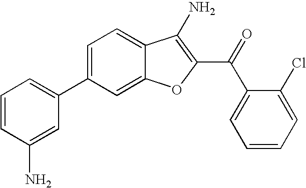 | R_{f} = 0.10, HEX/EtOAc = 70/30 | 363.0 | A-2 | comm | C-1 |
| 103 | 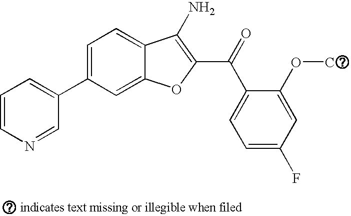 | R_{f} = 0.14 (50% EtOAc/HEX) RT = 2.50 | 363.4 | A-2 | comm | C-1 |
| 104 | 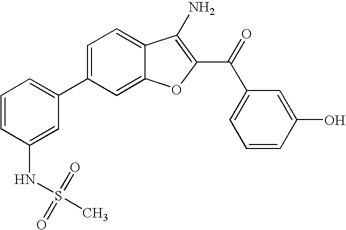 | R_{f} = 0.10, HEX/EtOAc = 50/50 | 423.0 | A-6 | comm | C-1 |
| 105 | 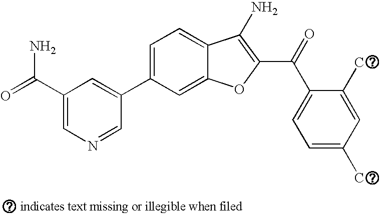 | R_{f} 0.11 (100% EtOAc) | 426.0 | comm | comm | C-2 |
| 106 | 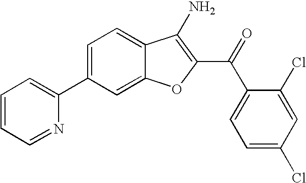 | R_{f} = 0.10 (25% EtOAc/HEX) | 383.0 | comm | comm | C-2 |
| 107 | 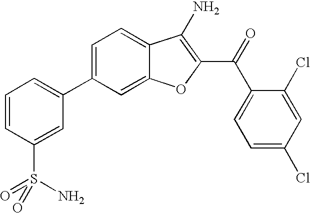 | R_{f} = 0.15 (SILICA, EtOAc:HEX, 4:6) | 461.0 | comm | comm | C-2 |
| 108 | 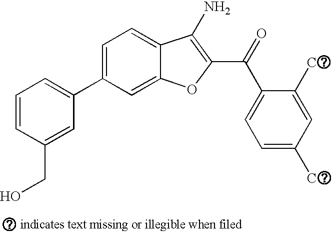 | R_{f} = 0.13 (SILICA, EtOAc/HEX, 3/7) | 412.0 | comm | comm | C-2 |
| 109 | 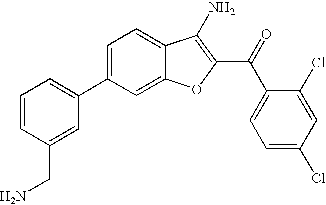 | R_{f} = 0.11 (SILICA, MeOH/CH₂Cl₂, 6/94) | 435 | comm | comm | C-2 |
| 110 | 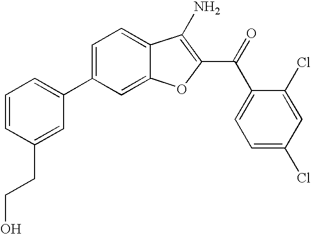 | R_{f} = 0.09 (SILICA, EtOAc/HEX, 3/7) | 426.0 | comm | comm | C-2 |
| 111 | 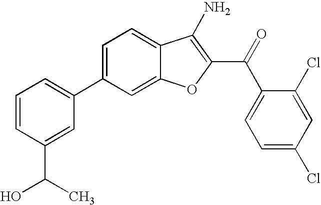 | R_{f} = 0.26 (SILICA, EtOAc/HEX, 3/7) | 426.0 | comm | comm | C-2 |
| 112 | 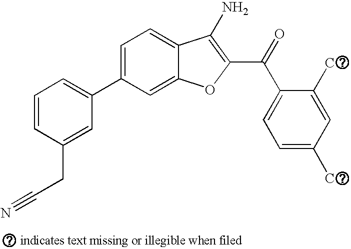 | R_{f} = 0.23 (SILICA, EtOAc/HEX, 3/7) | 421.0 | comm | comm | C-2 |
| 113 | 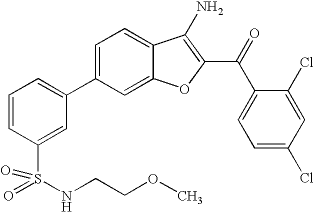 | R_{f} = 0.29 (SILICA, EtOAc/HEX, 3/7) | 519.0 | comm | H-11 | C-2 |
| 114 | 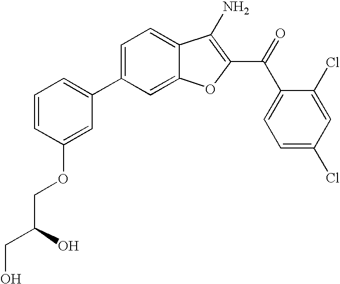 | R_{f} = 0.55, 100% EtOAc | 472/474 | comm | H-6 | C-2 |
| 115 | 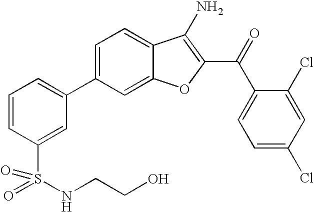 | R_{f} = 0.18 (SILICA, EtOAc/HEX, 6/4) | 505.0 | comm | H-11 | C-2 |
| 116 | 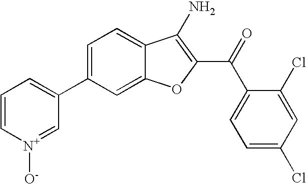 | R_{f} = 0.15 (10% MeOH/EtOAc) | 399.0 | comm | comm | C-2 |
| 117 | 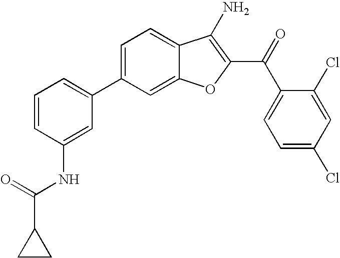 | R_{f} = 0.23, HEX/EtOAc = 70/30 | 465/467 | comm | H-10 | f |
| 118 | 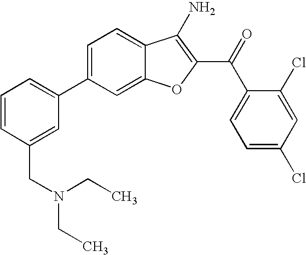 | R_{f} = 0.14 (SILICA, EtOAc/HEX, 1/1) | 467.0 | comm | H-12 | C-2 |
| 119 | 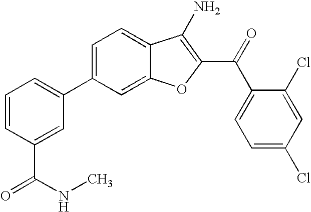 | R_{f} = 0.24 (SILICA, EtOAc/HEX, 3/2) | 439.0 | comm | H-13 | C-2 |
| 120 | 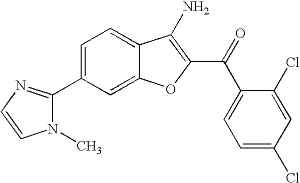 | R_{f} = 0.33 EtOAc) | 386.0 | comm | comm | C-2 |
| 121 | 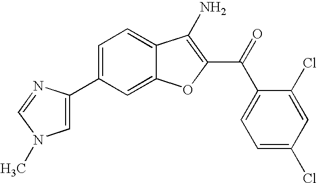 | R_{f} = 0.33 EtOAc) | 386.0 | comm | comm | C-2 |
| 122 | 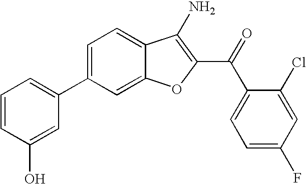 | R_{f} = 0.12, HEX/EtOAc = 70/30 | 382.0 | A-4 | comm | C-2 |
| 123 | 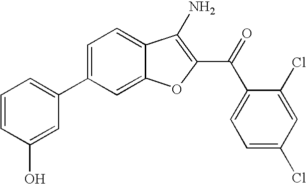 | R_{f} = 0.14, HEX/EtOAc = 70/30 | 398/400 | comm | comm | C-2 |
| 124 | 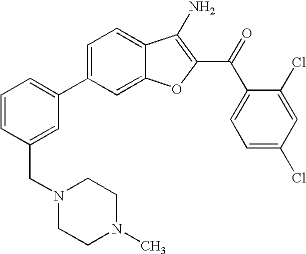 | R_{f} = 0.30 (SILICA, MeOH/CH₂Cl₂, 8/92) | 494.0 | comm | H-12 | C-2 |
| 125 | 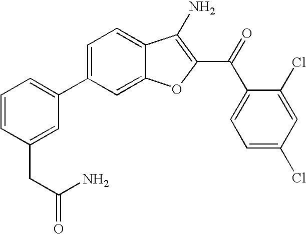 | R_{f} = 0.17 (SILICA, EtOAc/HEX, 4/1) | 439.0 | comm | H-14 | C-2 |
| 126 | 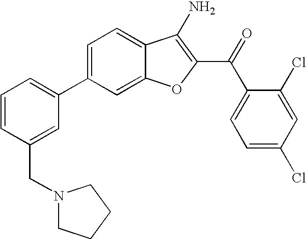 | R_{f} = 0.07 (SILICA, EtOAc/HEX, 7/3) | 465.0 | comm | H-12 | C-2 |
| 127 | 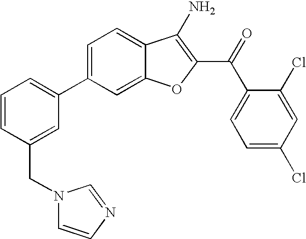 | R_{f} = 0.30 (SILICA, MeOH/CH₂Cl₂, 6/94) | 462.0 | comm | H-12 | C-2 |
| 128 | 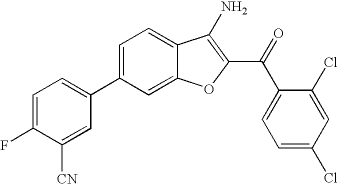 | R_{f} = 0.32 HEX/EtOAc = 70/30 | 425/427 | comm | comm | C-2 |
| 129 | 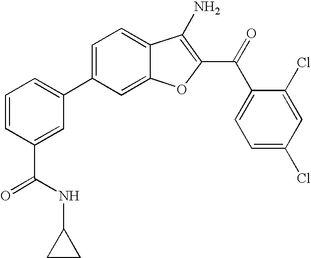 | R_{f} = 0.13 (SILICA, EtOAc/HEX, 4/6) | 465.0 | comm | H-13 | C-2 |
| 130 | 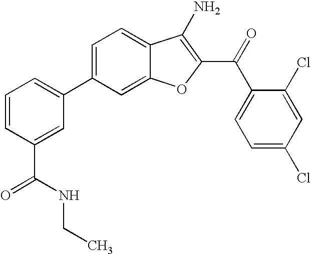 | R_{f} = 0.24 (SILICA, EtOAc/HEX, 4/6) | 453.0 | comm | H-13 | C-2 |
| 131 | 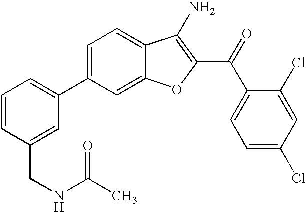 | R_{f} = 0.10 (SILICA, EtOAc/HEX, 6/4) | 453.0 | comm | H-4 | C-2 |
| 132 | 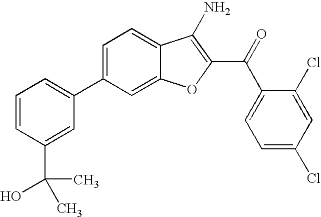 | R_{f} = 0.35 (SILICA, EtOAc/HEX, 4/6) | 440.0 | comm | H-15 | C-2 |
| 133 | 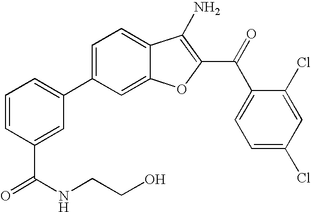 | R_{f} = 0.17 (SILICA, EtOAc/HEX, 8/1) | 469.0 | comm | H-13 | C-2 |
| 134 | 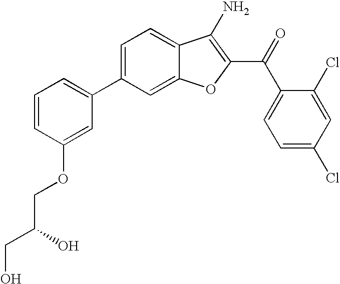 | R_{f} = 0.55, 100% EtOAc | 472/474 | comm | H-6 | C-2 |
| 135 | 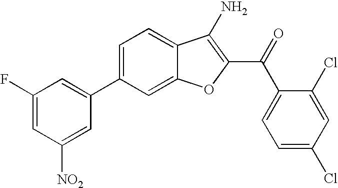 | R_{f} = 0.45, HEX/EtOAc = 70/30 | 445/447 | comm | comm | C-2 |
| 136 | 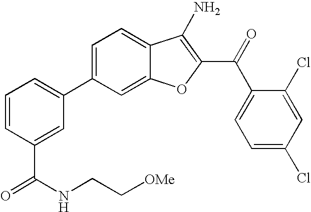 | R_{f} = 0.14 (SILICA, EtOAc/HEX, 1/1) | 483.0 | comm | H-13 | C-2 |
| 137 | 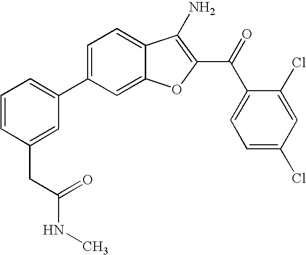 | R_{f} = 0.17 (SILICA, EtOAc/HEX, 7/3) | 453.0 | comm | H-13 | C-2 |
| 138 | 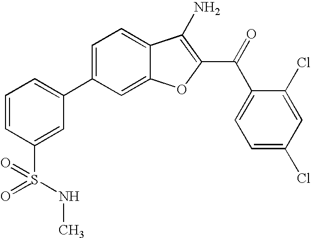 | R_{f} = 0.10 (SILICA, EtOAc/HEX, 3/7) | 475.0 | comm | H-11 | C-2 |
| 139 | 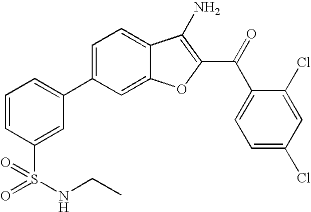 | R_{f} = 0.15 (SILICA, EtOAc/HEX, 3/7) | 489.0 | comm | H-11 | C-2 |
| 140 | 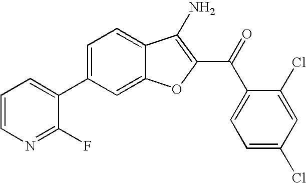 | R_{f} = 0.23, HEX/EtOAc = 70/30 | 401/403 | comm | H-7 | C-2 |
| 141 | 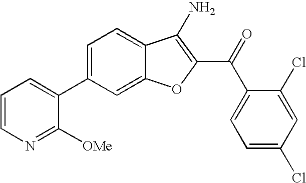 | R_{f} = 0.30, HEX/EtOAc = 70/30 | 413.0 | comm | H-8 | C-2 |
| 142 | 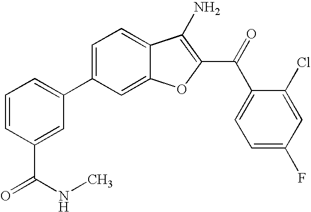 | R_{f} = 0.26 (SILICA, EtOAc/HEX, 3/2) | 423.0 | A-2 | H-13 | C-2 |
| 143 | 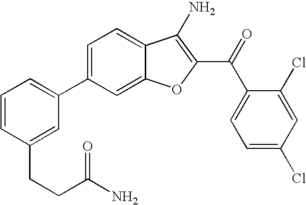 | R_{f} = 0.12 (SILICA, EtOAc/HEX, 4/1) | 453.0 | Comm | H-14 | C-2 |
| 144 | 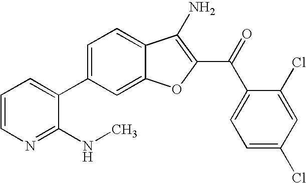 | R_{f} = 0.33, HEX/EtOAc = 70/30 | 412/414 | comm | H-9 | C-2 |
| 145 | 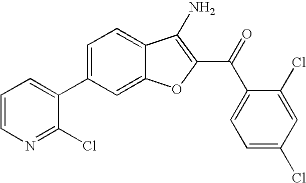 | R_{f} = 0.29, (40% EtOAc/HEX) | 419.0 | comm | comm | C-2 |

Footnotes:
*The following are the LCMS conditions: HPLC-electrospray mass spectra (HPLC ES-MS) were obtained using a Gilson HPLC system equipped with two Gilson 306 pumps, a Gilson 215 Autosampler, a Gilson diode array detector, a YMC Pro C-18 column (2 × 23 mm, 120 A), and a Micromass LCZ single quadrupole mass spectrometer with z-spray electrospray ionization. Spectra were scanned from 120-1000 amu over 2 seconds. ELSD (Evaporative Light Scattering Detector) data was also acquired as an analog channel. Gradient elution was used with Buffer A as 2% acetonitrile in water with 0.02% TFA and Buffer B as 2% water in Acetonitrile with 0.02% TFA at 1.5 mL/min. Samples were eluted as follows: 90% A for 0.5 min ramped to 95% B over 3.5 min and held at 95% B for 0.5 min and then the column is brought back to initial conditions over 0.1 min. Total run time is 4.8 min.
**comm means commercially available.

### General Method D: Preparation of Formula (I) Compounds via 6-Bromo-Benzofuran Intermediates (VIII) and Boronates (V) and (VI).

Alternative methods for the preparation of compounds of formula (I), are illustrated in the Reaction Schemes for General Method D-1 and D-2 and the Reaction Scheme for General Method D-3, below.

In General methods D-1 and D-2, the common intermediate, [(3-Amino-6-bromo-benzofuran-2-yl)-(2,4-dichloro-phenyl)-methanone (VIII) was used to prepare the 6-substituted benzofuran analogs with formula (I). Compound (VIII) was synthesized from 4-bromo-2-fluoro-benzonitrile using three simple chemical conversions (see experimental) as illustrated in the Reaction Scheme below. Palladium mediated coupling reactions between (VIII) and arylboronic acids or boronates (VI) afforded the desired compounds with the formula (I). Alternatively, 6-bromo-benzofuran (VIII) was converted to boronate (V), which was then used to prepare the desired compounds via palladium mediated coupling with arylhalides (VII).

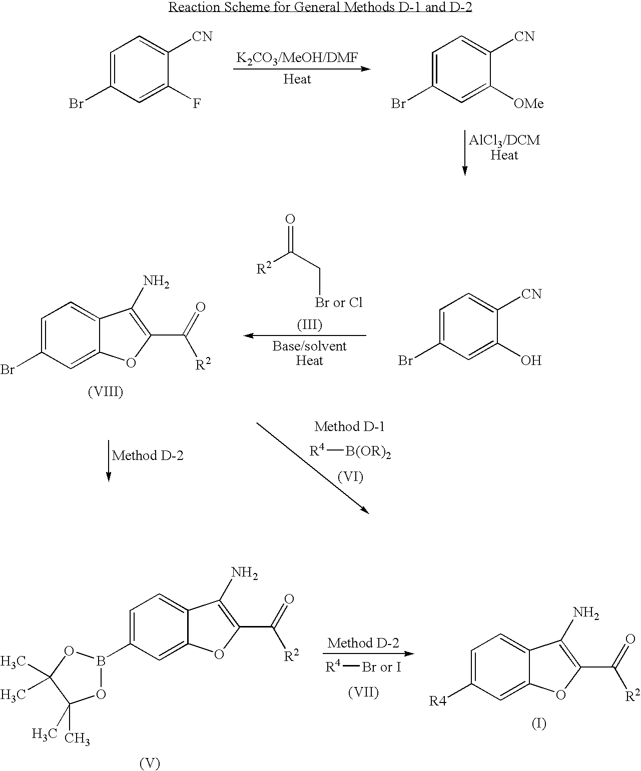

When R¹=low alkyl in formula (I), it was prepared according to General Method D-3. Intermediate (VIII) was acylated by the conventional methods followed by borane reduction to form intermediate (IX). Palladium mediated coupling reactions between (IX) and arylboronic acids or boronates (VI) afforded the desired compounds with the formula (I).

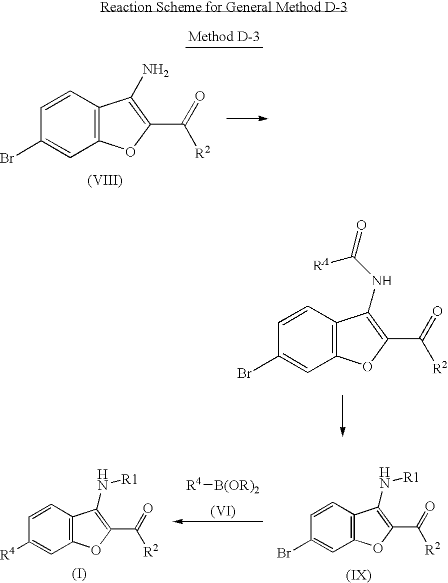

### Example 146

### Preparation of [(3-Amino-6-bromo-benzofuran-2-yl)-(2,4-dichloro-phenyl)-methanone

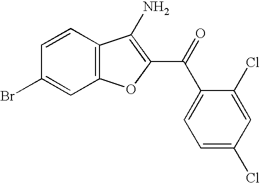

### Step 1: Preparation of the starting material: 4-bromo-2-methoxy-benzonitrile

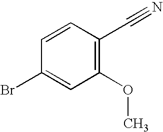

A mixture of 4-bromo-2-fluoro-benzonitrile (15.0 g, 75.0 mmol), methanol (30.4 mL, 350 mmol) and potassium carbonate (31.1 g, 225 mmol) in DMF (150 mL) was stirred under argon at 55° C. overnight. At this point TLC (100% methylene chloride) revealed no starting material, and the reaction mixture was poured into ether (300 mL) and water (150 mL). The layers were separated, and the organic layer was washed with water (150 mL) and brine (50 mL), dried over Mg₂SO₄, filtrated, and concentrated under reduced pressure, providing (15.2 g, 95.5%) of 4-bromo-2-methoxy-benzonitrile as a white solid. ¹H-NMR (CDCl₃) δ 7.41 (d, J=8.1 Hz, 1H), 7.16 (dd, J=8.1, 1.6 Hz, 1H), 7.13 (d, J=1.6 Hz, 1H), 3.93 (s, 3H); MS GC-MS (M⁺=211; RT=6.15 min).

### Step 2: Preparation of the intermediate: 4-bromo-2-hydroxy-benzonitrile

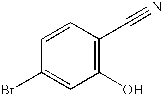

To a stirred solution of 4-bromo-2-methoxy-benzonitrile (4.60 g, 21.7 mmol) in methylene chloride (20 mL) was added aluminum chloride (14.5 g, 108 mmol). After stirring under an argon atmosphere for 10 min, more methylene chloride (30 mL) was added, and the mixture left to reflux under argon overnight. The reaction was then diluted with ethyl acetate, washed with water, brine, and dried over magnesium sulfate. The solvent was removed at reduced pressure, providing (4.09 g, 95.2%) of 4-bromo-2-hydroxy-benzonitrile as a slightly gray-colored product. ¹H-NMR (CDCl₃) δ 7.35 (d, J=8.4 Hz, 1H), 7.19 (d, J=1.4 Hz, 1H), 7.14 (dd, J=8.4, 1.4 Hz, 1H), 6.15 (s, 1H); TLC R_{f}=0.78 (50% ethyl acetate-hexane).

### Step 3: [(3-Amino-6-bromo-benzofuran-2-yl)-(2,4-dichloro-phenyl)-methanone

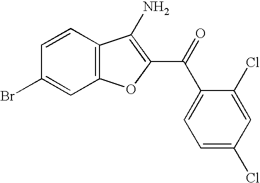

This compound was prepared from 4-bromo-2-hydroxy-benzonitrile (4.0 g, 20.3 mmol) in the manner described for [(3-amino-6-iodo-benzofuran-2-yl)-(2,4-dichloro-phenyl)-methanone (Example C-1 step 2), affording 6.1 g (78%) of [(3-amino-6-bromo-benzofuran-2-yl)-(2,4-dichloro-phenyl)-methanone as a yellow solid. ¹H-NMR (DMSO-d₆) δ 7.96 (d, J=8.3 Hz, 1H), 7.74 (d, J=1.6 Hz, 1H), 7.72 (dd, J=1.7 Hz, 1.0 Hz, 1H), 7.56-7.49 (m, 4H), 7.44, (dd, J=8.5 Hz, 1.7 Hz, 1H). MS LC-MS (MH⁺=386.1), LC MS RT: 3.68 min.

### Example 147

### Method D-1

### Palladium mediated coupling between [(3-amino-6-bromo-benzofuran-2-yl)-(2,4-dichloro-phenyl)-methanone and arylboronic acids or boronates

The exact procedures described in Example C-1 step 3 were followed except using [(3-amino-6-bromo-benzofuran-2-yl)-(2,4-dichloro-phenyl)-methanone (VIII) instead of [(3-amino-6-iodo-benzofuran-2-yl)-(2,4-dichloro-phenyl)-methanone (N). Similar reaction also can be found in Example D-3 step 2.

### Example 148

### Method D-2

### Preparation of [3-amino-6-(3-ethyl-phenyl)-benzofuran-2-yl]-(2,4-dichloro-phenyl)-methanone

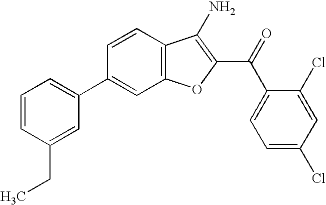

### Step 1: Preparation of [3-Amino-6-(4,4,5,5-tetramethyl-[1,3,2]dioxaborolan-2-yl)-benzofuran-2-yl]-(2,4-dichloro-phenyl)-methanone

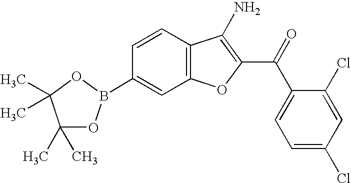

A mixture of [(3-amino-6-bromo-benzofuran-2-yl)-(2,4-dichloro-phenyl)-methanone (2.5 g, 6.5 mmol), potassium acetate (1.97 g, 19.5 mmol), bis(pinacolato)diboron (1.99 g, 7.79 mmol), in anhydrous DMSO was degassed under Ar for 30 min. Then Pd(dppf)₂Cl₂ (0.53 g, 0.65 mmol) was added, and the mixture degassed an additional 10 min. The reaction was then heated to 100° C. for 3.5 h. The reaction mixture was then poured into ethyl acetate and water. The organic layer was dried with magnesium sulfate, filtered and concentrated in vacuo. The crude residue was purified by silica column eluting with 25% ethyl acetate-hexane and providing 1.2 g (43%) of 3-amino-6-(4,4,5,5-tetramethyl-[1,3,2]dioxaborolan-2-yl)-benzofuran-2-yl]-(2,4-dichloro-phenyl)-methanone as a brown solid. ¹H-NMR (CDCl₂) δ 7.81 (s, 1H), 7.67 (d, J=7 Hz, 1H), 7.60 (d, J=7 Hz, 1H), 7.55-7.46 (m, 2H), 7.34, (dd, J=8.2 Hz, 2.0 Hz, 1H), 5.93 (bs, 2H), 1.33 (3, 12H). MS LC-MS (MH⁺=432.3, 434.2), LC MS RT: 3.95 min.

### Step 2: preparation of [3-amino-6-(3-ethyl-phenyl)-benzofuran-2-yl]-(2,4-dichloro-phenyl)-methanone

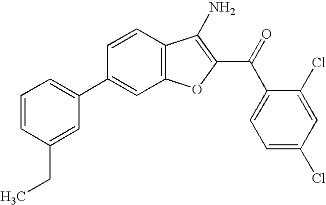

This compound was prepared from 1-bromo-3-ethyl-benzene (0.06 g, 0.30 mmol) using the manner described for [3-amino-6-(2-methyl-pyridydinyl)-1-benzofuran-2-yl] (2,4-dichlorophenyl)methanone (Example C-2 step 2), affording 35.1 mg (37%) of [3-amino-6-(3-ethyl-phenyl)-benzofuran-2-yl]-(2,4-dichloro-phenyl)-methanone as a yellow solid. ¹H-NMR (CDCl₃) δ 7.67 (dd, J=8.1 Hz, 1H), 7.56-7.53 (m, 1H), 7.55-7.49 (m, 3H), 7.45-7.34 (m, 4H), 7.25-7.21 (dm, J=4 Hz, 1H), 6.04 (bs, 2H), 2.72 (q, J=6 Hz, 2H), 1.28 (t, J=6 Hz, 3H). MS LC-MS (MH⁺=410.3, 412.1), LC MS RT: 4.17 min.

### Example 149

### Method D-3

### Preparation of N-{3-[2-(2,4-Dichloro-benzoyl)-3-methylamino-benzofuran-6-yl]-phenyl}-acetamide

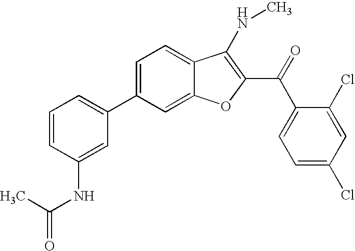

### Step 1: Preparation of intermediate (6-Bromo-3-methylamino-benzofuran-2-yl)-(2,4-dichloro-phenyl)-methanone

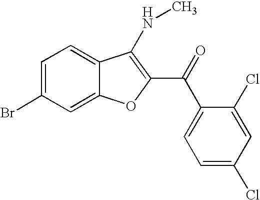

Acetic formic anhydride was made by the following method: Formic acid (1.5 mL, 39 mmol) was added dropwise into acetic anhydride (3 mL, 32 mmol) in a 250 mL flask in an ice bath, followed by gentle heating at 60° C. for 2 h. To the cooled flask of acetic formic anhydride (2.5 eq) was added the solution of (3-Amino-6-bromo-benzofuran-2-yl)-(2,4-dichloro-phenyl)-methanone prepared according to method D (5 g, 13 mmol, 1 eq) in 20 mL anhydrous THF. The reaction was refluxed at 70° C. for 40 h. Lots of solid precipitated while cooling the reaction. The white solid was filtered out, washed with THF and dried in vacuum oven. The product was thus formed was suspended in 40 mL THF and borane methyl sulfide (3 mL, 32 mmol, 2.5 eq) was added dropwise in an ice bath. The reaction mixture was stirred in the ice bath for 3 h, 10 mL methanol was added, stirred for another 30 min. The reaction mixture was evaporated to give green sticky material. White solid was formed while adding EtOAc to the mixture and the solid was removed by filtration. The filtrate was concentrated and the residue was purified by MPLC (Biotage). 700 mg (14%) of 6-Bromo-3-methylamino-benzofuran-2-yl)-(2,4-dichloro-phenyl)-methanone was obtained as a yellow solid. ¹H-NMR (CDCl₃) δ 7.98 (broad, m, 1H), 7.82 (d, J=8 Hz, 1H), 7.52 (d, J=2 Hz, 1H), 7.48 (d, J=2 Hz, 1H), 7.44 (d, J=8 Hz, 1H), 7.35 (t, J=2 Hz, 1H), 7.33 (t, J=2 Hz, 1H), 2.40 (s, 3H). MS LC-MS MH⁺=398/400/402; R_{f}=0.72, 50% EtOAc-HEX.

### Step 2: Preparation of N-{3-[2-(2,4-Dichloro-benzoyl)-3-methylamino-benzofuran-6-yl]-phenyl}-acetamide

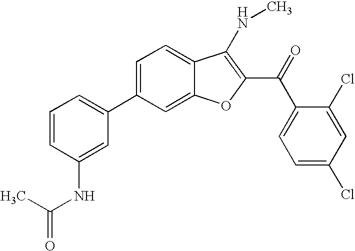

A solution of (6-Bromo-3-methylamino-benzofuran-2-yl)-(2,4-dichloro-phenyl)-methanone (100 mg, 0.25 mmol) in ethylene glycol dimethyl ether (1 mL) was degassed with argon for 10 min. At this time degassed 3-acetamidobenzene boronic acid (49 mg, 0.28 mmol, 1.1 eq) in ethylene glycol dimethyl ether (4 mL) was added followed by [1,1′-bis(diphenylphosphino)-ferrocene]dichloro-palladium(II) complex (20 mg, 0.03 mmol, 0.1 eq) and 2M aqueous sodium carbonate (0.63 mL, 1.25 mmol, 5 eq). The reaction was bubbled with argon for 10 min and it was heated to 80° C. for 5 h. The reaction was diluted with EtOAc, washed with water and brine. The organic layer was dried in vacuo. Purification using preparative TLC gave 66.4 mg (59%) N-{3-[2-(2,4-dichloro-benzoyl)-3-methylamino-benzofuran-6-yl]phenyl}-acetamide as a yellow solid. ¹H-NMR (CDCl₃) δ 8.0 (broad, q, J=5.6 Hz, 1H), 7.91 (d, J=8.4 Hz, 1H), 7.63 (broad, s, 1H), 7.50 to 7.33 (m, 8H), 3.42 (d, J=5.6 Hz, 3H), 2.21 (s, 3H). MS LC-MS MH⁺=453.2/455.2; R_{f}=0.13, 50% EtOAc-HEX.

The other compounds in Table 3 can be prepared in like manner as described above by choosing the appropriate starting materials that are readily available and/or the synthesis of which is taught herein, and using the process described above or other standard chemical processes known in the art.

TABLE 3
Examples Synthesized using Method D

| | | | | | Synthesis | |
|---|---|---|---|---|---|---|
| | | R_{f} (TLC solvent) | LC/MS | Synthesis | of (VI) or | Synth. |
| Example | Structure | Or RT (min)* | ([M + H]+) | of (III)** | (VII)** | of (I) |
| 150 | 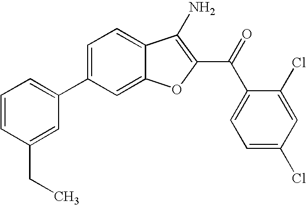 | RT = 4.17 | 410.3 | comm | comm | D-2 |
| 151 | 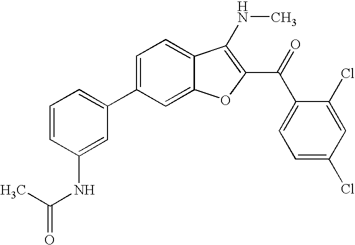 | R_{f} = 0.13 @ 50/50 EtOAc/HEX | 453.2/455.2 | comm | comm | D-3 |
| 152 | 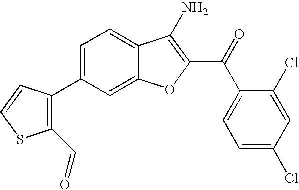 | Rt = 3.94 | 416.1 | comm | comm | D-1 |
| 153 | 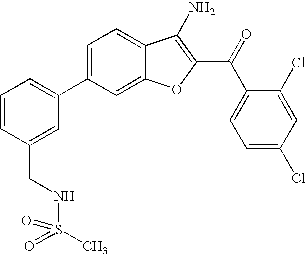 | R_{f} = 0.09 (50% EtOAc/HEX) | 489.0 | comm | H-4 | D-2 |
| 154 | 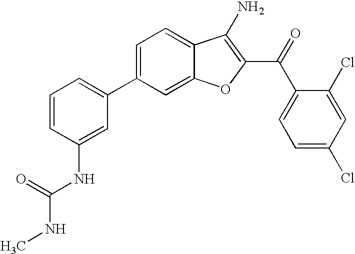 | R_{f} = 0.19 (75% EtOAc/HEX) | 454.0 | comm | H-5 | D-2 |
| 155 | 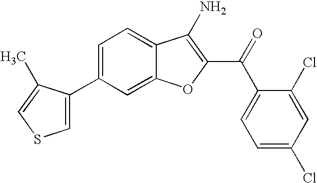 | RT = 4.33 | 402.1 | comm | comm | D-2 |
| 156 | 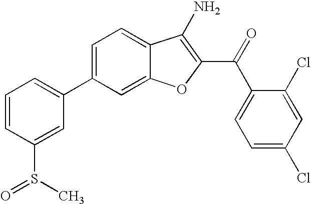 | RT = 3.54 | 444.1 | comm | H-3 | D-2 |
| 157 | 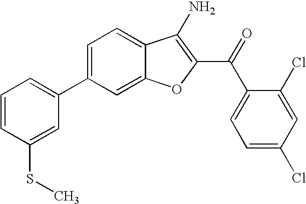 | RT = 4.41 | 428.2 | comm | comm | D-2 |
| 158 | 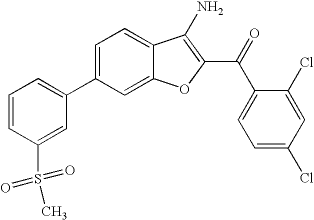 | RT = 3.78 | 460.1 | comm | H-3 | D-2 |
| 159 | 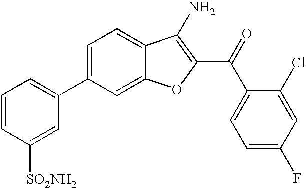 | RT = 2.96 | 445.1 | A-2 | comm | D-2 |
| 160 | 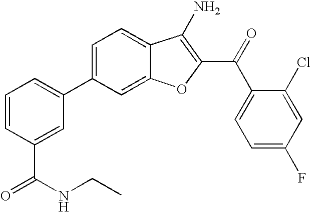 | RT = 3.58 | 437.2 | A-2 | comm | D-2 |
| 161 | 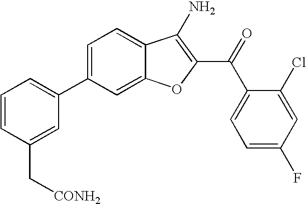 | RT = 2.89 | 423.2 | A-2 | comm | D-2 |
| 162 | 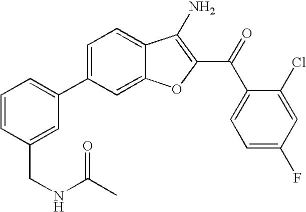 | RT = 3.00 | 437.2 | A-2 | comm | D-2 |
| 163 | 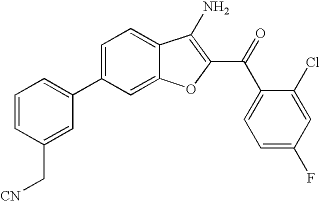 | RT = 3.82 | 405.2 | A-2 | comm | D-2 |
| 164 | 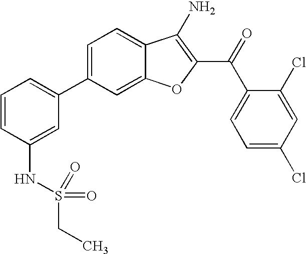 | R_{f} = 0.21 (50% EtOAc/HEX); CONTAINS ~5% IMPURITY (BIS- ADDUCT D) | 489.0 | comm | H-4 | D-2 |
| 165 | 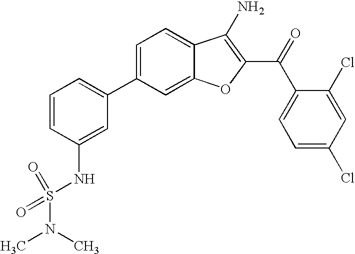 | R_{f} = 0.3 (50% EtOAc/HEX); CONTAINS ~5% OF THE BIS- ADDUCT D. | 504.0 | comm | H-4 | D-2 |
| 166 | 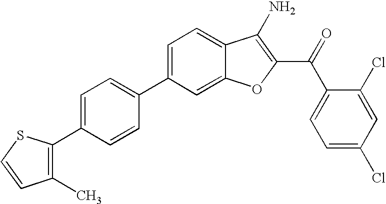 | RT = 4.37 | 402.1 | comm | comm | D-2 |
| 167 | 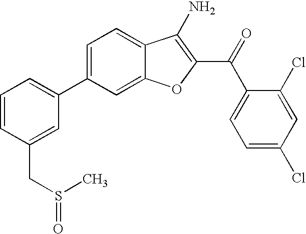 | RT = 3.06 | 458.0 | comm | H-1, H-3 | D-2 |
| 168 | 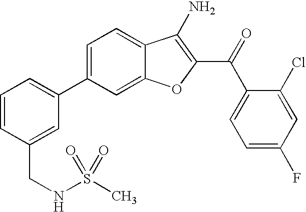 | R_{f} = 0.08 (50% WtOAc/HEX) | 473.0 | A-2 | H-4 | D-2 |
| 169 | 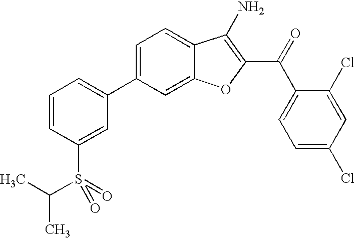 | RT = 4.03 | 488.2 | comm | H-2, H-3 | D-2 |
| 170 | 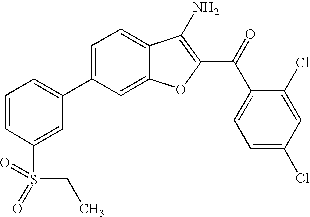 | RT = 3.91 | 474.1 | comm | H-2, H-3 | D-2 |
| 171 | 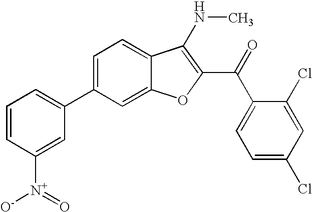 | R_{f} 0.59 @ 50/50 EtOAc/HEX | 441.1/443.1 | comm | comm | D-3 |
| 172 | 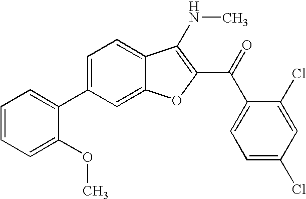 | R_{f} 0.70 @ 50/50 EtOAc/HEX | 426.2/428.2 | comm | comm | D-3 |
| 173 | 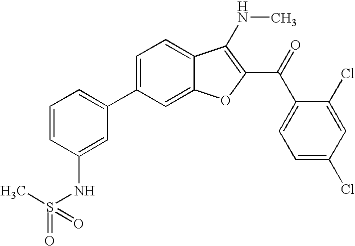 | R_{f} = 0.28 @ 50/50 EtOAc/HEX | 489.1/491.1 | comm | comm | D-3 |
| 174 | 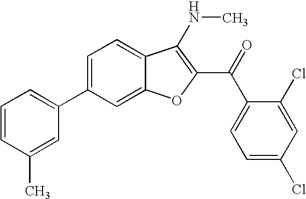 | R_{f} = 0.76 @ 50/50 EtOAc/HEX | 410.1/412.1 | comm | comm | D-3 |
| 175 | 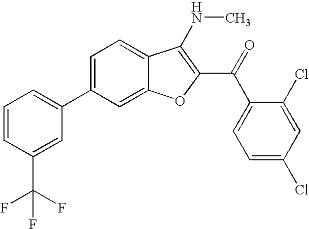 | R_{f} = 0.74 @ 50/50 EtOAc/HEX | 464.1/466.1 | comm | comm | D-3 |

Footnotes:
*The following are the LCMS conditions: HPLC-electrospray mass spectra (HPLC ES-MS) were obtained using a Gilson HPLC system equipped with two Gilson 306 pumps, a Gilson 215 Autosampler, a Gilson diode array detector, a YMC Pro C-18 column (2 × 23 mm, 120 A), and a Micromass LCZ single quadrupole mass spectrometer with z-spray electrospray ionization. Spectra were scanned from 120-1000 amu over 2 seconds. ELSD (Evaporative Light Scattering Detector) data was also acquired as an analog channel. Gradient elution was used with Buffer A as 2% acetonitrile in water with 0.02% TFA and Buffer B as 2% water in Acetonitrile with 0.02% TFA at 1.5 mL/min. Samples were eluted as follows: 90% A for 0.5 min ramped to 95% B over 3.5 min and held at 95% B for 0.5 min and then the column is brought back to initial conditions over 0.1 min. Total run time is 4.8 min.
**comm means commercially available.

### General Method E: Preparation of N-Heterocyclic-Substituted Benzofurans

Methods E-1 to E-4 as illustrated in the General Reaction Schemes below, were the means used to prepare examples in this invention where R⁴ substituents are attached to the benzofuran core through a nitrogen atom. Examples E-1 through E-4 describe various ways to prepare the properly substituted 2-cyano phenols (intermediates XI or (XIII). The condensation of (XI) or (XIII) with 1-aryl-2-haloethanone (III) under basic conditions (such as cesium carbonate, potassium carbonate, sodium carbonate, DBU), in a solvent such as DMF, MeCN at temperatures between room temperature to 100° C. to give the desired products with formula (I).

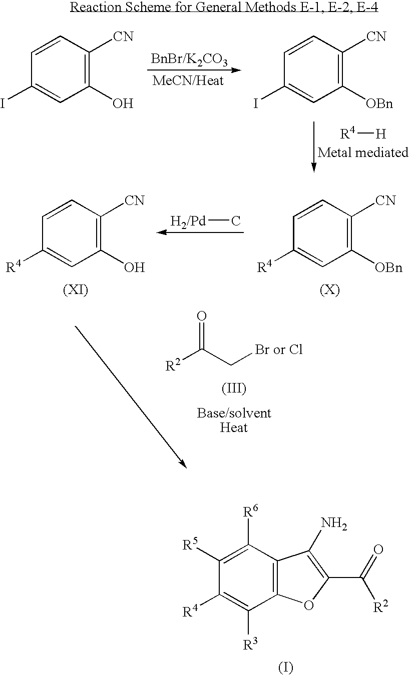

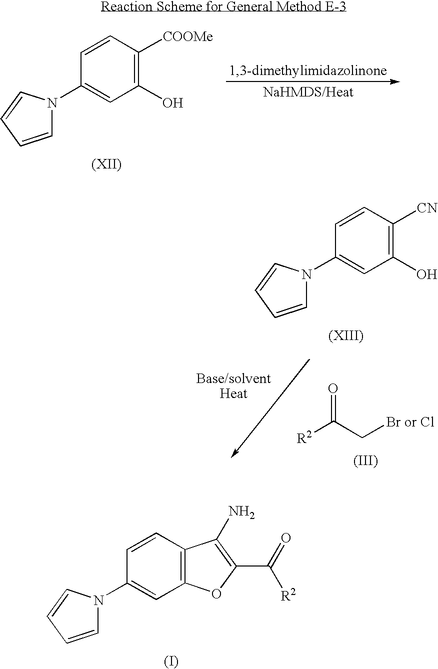

### Example 176

### Method E-1

### Preparation of 3-[3-amino-2-(2,4-dichlorobenzoyl)-1-benzofuran-6-yl]-1,3-oxazolidin-2-one

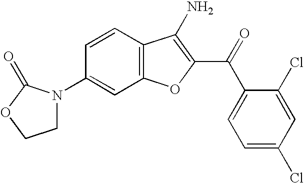

### Step 1: Preparation of starting material: 2-(benzyloxy)-4-iodobenzonitrile

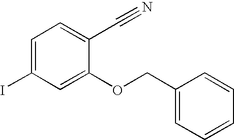

A mixture of 2-cyano-5-iodophenol (963 mg, 3.93 mmol), benzyl bromide (0.51 mL, 4.32 mmol, 1.1 eq), and potassium carbonate (597.5 mg, 4.32 mmol, 1.1 eq) in anhydrous acetonitrile was stirred at reflux under argon for 17 h. The resulting reaction was poured into ethyl acetate (300 mL) and water (150 mL). The ethyl acetate layer was washed with saturated aqueous ammonium chloride, water, and brine. The organic layer was then dried over sodium sulfate, filtered, and evaporated under reduced pressure to give a white solid (1.31 g, 99.5%). GC-MS (ES MH⁺=336); TLC R_{f}=0.27 (5% ethyl acetate-hexane).

### Step 2: Preparation of starting material: 2-(benzyloxy)-4-(2-oxo-1,3-oxazolidin-3-yl)benzonitrile

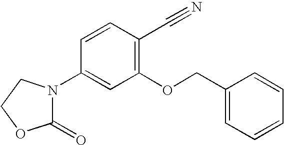

A solution of 2-(benzyloxy)-4-iodobenzonitrile (400 mg, 1.19 mmol), 2-oxazolidone (519.6 mg, 5.97 mmol, 5.0 eq), copper (140.3 mg, 2.21 mmol, 1.85 eq), potassium carbonate (240.8 mg, 1.74 mmol, 1.46 eq), and potassium iodide (309.1 mg, 1.86 mmol, 1.56 eq) in anhydrous N,N-dimethylformamide (4.8 mL) was stirred at 150° C. for 19 h. The resultant orange reaction was diluted with ethyl acetate, washed with water, brine, and dried over magnesium sulfate. The solvent was removed at reduced pressure and purified on the MPLC (Biotage) eluted with 4:1:5 v/v ethyl acetate-dichloromethane-hexane to afford 142.6 mg (40.6%) of the product. ¹H-NMR (DMSO-d₆) δ 7.73 (d, J=9 Hz, 1H), 7.54 (d, J=2.4 Hz, 1H), 7.49 to 7.34 (m, 5H), 7.25 (dd, J=8.7 Hz, 2.1 Hz, 1H), 5.27 (s, 2H), 4.45 (t, J=8.1 Hz, 2H), 4.07 (t, J=8.7 Hz, 2H); LC-MS (ES MH⁺=294.9, RT=2.82 min).

### Step 3: Preparation of 2-hydroxy-4-(2-oxo-1,3-oxazolidin-3-yl)benzonitrile

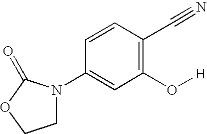

To a dry flask charged with 10% Pd/C (14.0 mg, 0.05 mmol, 0.1 eq) was added a solution of 2-(benzyloxy)-4-(2-oxo-1,3-oxazolidin-3-yl)benzonitrile (140 mg, 0.48 mmol) in 1:1 v/v tetrahydrofuran-ethanol (16 mL). The reaction mixture was hydrogenated under an atmosphere of hydrogen supplied by an attached balloon for 16 h. The reaction was filtered through a pad of celite, and the filtrate was concentrated to afford 90.5 mg (93%) of a white solid. ¹H-NMR (DMSO-d₆) δ 7.53 (d, J=8.4 Hz, 1H), 7.31 (s, 1H), 6.95 (dd, J=8.7 Hz, 1.8 Hz, 1H), 4.40 (t, J=8.7 Hz, 2H), 4.00 (t, J=8.4 Hz, 2H), 3.25 (broad s, 1H); TLC R_{f}=0.14 (75% ethyl acetate-hexane)

### Step 4: Preparation of 3-[3-amino-2-(2,4-dichlorobenzoyl)-1-benzofuran-6-yl]-1,3-oxazolidin-2-one

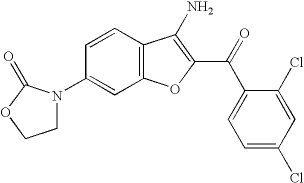

To a stirred solution of 2-hydroxy-4-(2-oxo-1,3-oxazolidin-3-yl)benzonitrile (62 mg, 0.30 mmol) and 2,2′,4′-trichloroacetophenone (101.8 mg, 0.46 mmol, 1.5 eq) in anhydrous N,N-dimethylformamide (3.0 mL) was added potassium carbonate (63.0 mg, 0.46 mmol, 1.5 eq), and the orange reaction mixture was stirred at 80° C. for 16 h. The resulting dark wine color reaction was poured into ethyl acetate (100 mL) and water (50 mL). The ethyl acetate layer was washed with saturated aqueous ammonium chloride, water, and brine. The organic layer was then dried over sodium sulfate, filtered, and evaporated under reduced pressure. The crude product was purified on the MPLC (Biotage) eluted with 60% ethyl acetate-hexane. Crystallization from dichloromethane-hexane afforded the benzofuran as an orange solid (43 mg, 36.2%). ¹H-NMR (DMSO-d₆) δ 8.01 (d, J=6.0 Hz, 1H), 7.93 (s, 1H), 7.73 (dd, J=1.8, 0.9 Hz, 1H), 7.63 (dd, J=8.7, 1.8 Hz, 1H), 7.55 to 7.52 (m, 4H), 4.43 (t, J=8.7 Hz, 2H), 4.07 (t, J=8.7 Hz, 2H); LC-MS (ES MH⁺=391/393, RT=3.00 min.).

### Example 177

### Method E-2

### Preparation of (3-Amino-6-morpholin-4-yl-benzofuran-2-yl)-(2,4-dichloro-phenyl)-methanone

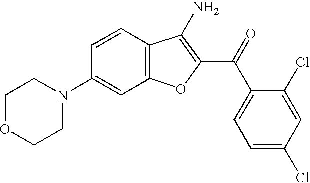

### Step 1: 2-(benzyloxy)-4-(morpholin-4-yl)benzonitrile

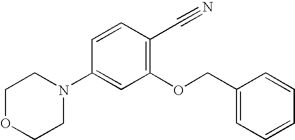

A solution of 2-(benzyloxy)-4-iodobenzonitrile (350 mg, 1.04 mmol), tris(dibenzylideneacetone)dipalladium(0) (95.6 mg, 0.10 mmol, 0.1 eq), tritolyl phosphate (95.3 mg, 0.31 mmol, 0.3 eq), potassium tert-butoxide (281.0 mg, 2.92 mmol, 2.8 eq) in anhydrous dioxane (5.2 mL) was degassed under argon. After 20 min, morpholine (0.22 mL, 2.51 mmol, 2.4 eq) was added, and the reaction mixture was stirred at 90° C. for 4 h. The reaction was diluted with ethyl acetate, washed with water, brine, and dried over magnesium sulfate. The solvent was removed at reduced pressure, and the crude material was purified on the MPLC (Biotage) eluted with 30% ethyl acetate/hexane to afford 220.5 mg (71.7%) of the product. ¹H-NMR (DMSO-d₆) [symbol] 7.48 to 7.33 (m, 6H), 6.70 (d, J=2.1 Hz, 1H), 6.59 (dd, J=8.7 Hz, 2.1 Hz, 1H), 5.24 (s, 2H), 3.70 (t, J=5.1 Hz, 4H), 3.29 (t, J=5.1 Hz, 4H); LC-MS (ES MH⁺=295, RT=3.09 min); R_{f}=0.17 (30% ethyl acetate-hexane).

### Step 2: Preparation of 2-hydroxy-4-(morpholin-4-yl)benzonitrile

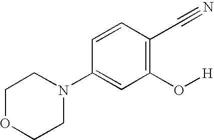

To a dry flask charged with 10% Pd/C (25.0 mg, 0.08 mmol, 0.1 eq) was added a solution of 2-(benzyloxy)-4-(morpholin-4-yl)benzonitrile (250 mg, 0.85 mmol) in 1:1 v/v ethyl acetate-ethanol (8.5 mL). The reaction mixture was hydrogenated under an atmosphere of hydrogen supplied by an attached balloon for 16 h. The reaction was filtered through a pad of celite, and the filtrate was concentrated. Crystallization from dichloromethane-hexanes gave 164.2 mg (94.7%) of a white solid. ¹H-NMR (DMSO-d₆) δ 10.64 (broad s, 1H), 7.33 (d, J=9.0 Hz, 1H), 6.49 (dd, J=9.0 Hz, 2.1 Hz, 1H), 6.34 (d, J=2.1 Hz, 1H), 3.68 (t, J=5.1 Hz, 4H), 3.15 (t, J=5.1H, 4H); LC-MS (ES MH⁺=205, RT=2.01 min); R_{f}=0.21 (50% ethyl acetate-hexane).

### Step 3: Preparation of (3-Amino-6-morpholin-4-yl-benzofuran-2-yl)-(2,4-dichloro-phenyl)-methanone

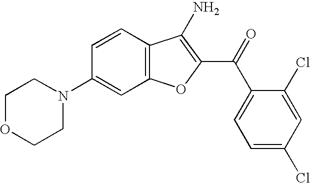

To a stirred solution of 2-hydroxy-4-(morpholin-4-yl)benzonitrile (75 mg, 0.37 mmol) and 2,2′,4′-trichloroacetophenone (123.1 mg, 0.55 mmol, 1.5 eq) in anhydrous N,N-dimethylformamide (3.7 mL) was added potassium carbonate (76.1 mg, 0.46 mmol, 1.5 eq), and the orange reaction mixture was stirred at 80° C. for 17 h. The resulting dark wine color reaction was poured into ethyl acetate (100 mL) and water (50 mL). The ethyl acetate layer was washed with saturated aqueous ammonium chloride, water, and brine. The organic layer was then dried over sodium sulfate, filtered, and evaporated under reduced pressure. The crude product was purified on the MPLC (Biotage) eluted with 30% ethyl acetate-hexane. Crystallization from ether-hexane afforded the benzofuran as a yellow solid (37 mg, 25.8%). ¹H-NMR (DMSO-d₆) δ 7.80 (d, J=9.3 Hz, 1H), 7.70 (m, 1H), 7.75 (m, 2H), 7.43 (broad s, 2H), 6.98 (dd, J=9 Hz, 1.8 Hz, 1H), 6.78 (d, J=1.8 Hz, 1H), 3.69 (t, J=4.5 Hz, 4H), 3.18 (t, J=4.5 Hz, 4H); LC-MS (ES MH⁺=391/393, RT=3.11 min).

### Example 178

### Method E-3

### Preparation of (3-Amino-6-pyrrol-1-yl-benzofuran-2-yl)-(2,4-dichloro-phenyl)-methanone

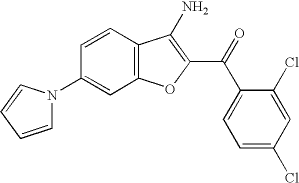

### Step 1: Preparation of starting material: 2-hydroxy-4-(1H-pyrrol-1-yl)benzonitrile

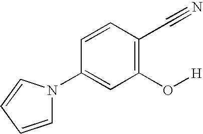

In a sealed tube was added methyl 2-hydroxy-4-(1H-pyrrol-1-yl)benzenecarboxylate (960 mg, 4.42 mmol), 1M sodium bis(trimethylsilyl)amide in THF (7.1 mL, 7.1 mmol, 1.6 eq), and 1,3-dimethylimidazolinone (1.77 mL), and the reaction mixture was heated to 185° C. for 17 h. The cooled reaction was quenched with 10% aqueous HCl solution and poured into ethyl acetate (200 mL) and water (100 mL). The ethyl acetate layer was washed with water and brine, dried over sodium sulfate, filtered, and evaporated under reduced pressure. The crude product was purified on the MPLC (Biotage) eluted with 25% ethyl acetate-hexane to give a white solid (585 mg, 71.9%). ¹H-NMR (DMSO-d₆) δ 11.36 (s, 1H), 7.66 (d, =8.4 Hz, 1H), 7.34 (t, J=2.4 Hz, 2H), 7.16 (dd, J=8.4 Hz, 2.4 Hz, 1H), 7.05 (d, J=1.8 Hz, 1H), 6.29 (t, J=2.4 Hz, 2H); R_{f}=0.18 (25% ethyl acetate-hexane).

### Step 2: Preparation of the title compound: (3-Amino-6-pyrrol-1-yl-benzofuran-2-yl)-(2,4-dichloro-phenyl)-methanone

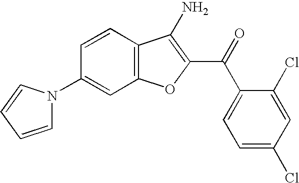

To a stirred solution of 2-hydroxy-4-(1H-pyrrol-1-yl)benzonitrile (60 mg, 0.33 mmol) and 2, 2′,4′-trichloroacetophenone (109.2 mg, 0.49 mmol, 1.5 eq) in anhydrous N,N-dimethylformamide (3.2 mL) was added potassium carbonate (67.5 mg, 0.49 mmol, 1.5 eq), and the orange reaction mixture was stirred at 80° C. for 16 h. The resulting dark wine color reaction was poured into ethyl acetate (100 mL) and water (50 mL). The ethyl acetate layer was washed with saturated aqueous ammonium chloride, water, and brine. The organic layer was then dried over sodium sulfate, filtered, and evaporated under reduced pressure. The crude product was purified on the MPLC (Biotage) eluted with 25% ethyl acetate-hexane. Crystallization from dichloromethane-hexane afforded the benzofuran as a yellow solid (89.0 mg, 73.6%). ¹H-NMR (DMSO-d₆) δ 8.08 (d, J=8.7 Hz, 1H), 7.73 (d, J=10.0 Hz, 2H), 7.60 to 7.53 (m, 5H), 7.48 (t, J=2.4 Hz, 2H), 6.26 (t, J=2.1 Hz, 2H); LC-MS (ES MH⁺=371, RT=3.74 min).

### Example 179

### Method E-4

### Preparation of (3-Amino-6-imidazol-1-yl-benzofuran-2-yl)-(2,4-dichloro-phenyl)-methanone

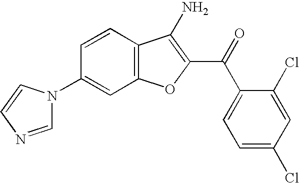

### Step 1: Preparation of starting material: 2-benzyloxy-4-(imidazol-1-yl)benzonitrile

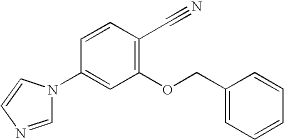

A mixture of 2-benzyloxy-4-iodo-benzonitrile (500 mg, 1.49 mmol), imidazole (152.3 mg, 2.24 mmol, 1.5 eq), cesium carbonate (534.7 mg, 1.64 mmol, 1.1 eq), copper (II) triflate (75.0 mg, 0.15 mmol, 0.1 eq), 1,10-phenanthroline (269 mg, 1.49 mmol, 1.0 eq), and trans, trans-dibenzylideneacetone (95.6 mg, 0.10 mmol, 0.1 eq) in anhydrous xylenes (6.0 mL) was sonicated for 2 min, and the reaction mixture was stirred at 110° C. for 19 h. The reaction was diluted with ethyl acetate, washed with water, brine, and dried over sodium sulfate. The solvent was removed at reduced pressure, and the crude material was purified on the MPLC (Biotage) eluted with 60% followed by 100% ethyl acetate. Crystallization from ethyl acetate-hexane afforded 240 mg (58.4%) of the product. ¹H-NMR (DMSO-d₆) δ 8.47 (t, J=0.9 Hz, 1H), 1.93 (t, J=1.5 Hz, 1H), 7.90 (d, J=8.4 Hz, 1H), 7.65 (d, J=2.1 Hz, 1H), 7.52 to 7.36 (m, 6H), 7.15 (t, J=1.5 Hz, 1H), 5.39 (s, 2H); LC-MS (ES MH⁺=276, RT=2.07 min); R_{f}=0.13 (75% ethyl acetate-hexane).

### Step 2: Preparation of starting material: 2-hydroxy-4-(imidazol-1-yl)benzonitrile

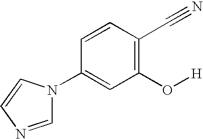

To a dry flask charged with 10% Pd/C (20.0 mg, 0.07 mmol, 0.1 eq) was added a solution of 2-benzyloxy-4-(imidazol-1-yl)benzonitrile (200 mg, 0.73 mmol) in 1:1 v/v ethyl acetate-ethanol (7.3 mL). The reaction mixture was hydrogenated under an atmosphere of hydrogen supplied by an attached balloon for 16 h. The reaction was filtered through a pad of celite, and the filtrate was concentrated. Recrystallization from ethyl acetate-hexane gave 120 mg (89.2%) of a white solid. ¹H-NMR (DMSO-d₆) δ 8.23 (s, 1H), 7.68 (s, 1H), 7.65 (d, J=8.1 Hz, 1H), 7.09 (s, 1H), 7.06 to 6.98 (m, 3H); R_{f}=0.13 (20% methanol-ethyl acetate).

### Step 3: Preparation of the title compound: (3-Amino-6-imidazol-1-yl-benzofuran-2-yl)-(2,4-dichloro-phenyl)-methanone

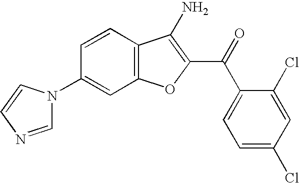

To a stirred solution of 2-hydroxy-4-(imidazol-1-yl)benzonitrile (60 mg, 0.32 mmol) and 2, 2′,4′-trichloroacetophenone (108.6 mg, 0.49 mmol, 1.5 eq) in anhydrous N,N-dimethylformamide (3.2 mL) was added potassium carbonate (67.2 mg, 0.49 mmol, 1.5 eq), and the orange reaction mixture was stirred at 80° C. for 16 h. The resulting dark wine color reaction was poured into ethyl acetate (100 mL) and water (50 mL). The ethyl acetate layer was washed with saturated aqueous ammonium chloride, water, and brine. The organic layer was then dried over sodium sulfate, filtered, and evaporated under reduced pressure. The crude product was purified on the MPLC (Biotage) eluted with 75% ethyl acetate-hexane followed by 100% ethyl acetate. Crystallization from dichloromethane-hexane afforded the benzofuran as an orange solid (32.5 mg, 27.0%). ¹H-NMR (DMSO-d₆) δ 8.37 (s, 1H), 8.16 (d, J=8.4 Hz, 1H), 7.86 (d, J=1.8 Hz, 1H), 7.84 (t, J=1.3 Hz, 1H), 7.76 (d, J=1.5 Hz, 1H), 7.65 (dd, J=8.4 Hz, J=1.8 Hz, 1H), 7.58 to 7.57 (m, 4H), 7.11 (s, 1H); LC-MS (ES MH⁺=372, RT=2.37 min).

The other compounds in Table 4 can be prepared in like manner as described above by choosing the appropriate starting materials that are readily available and/or the synthesis of which is taught herein, and using the process described above or other standard chemical processes known in the art.

TABLE 4
Examples Synthesized using Method E

| Exam- | | R_{f} (TLC solvent) | LC/MS | Synthesis | Synthesis | Synthesis |
|---|---|---|---|---|---|---|
| ple | Structure | Or RT (min)* | ([M + H]+) | of (III)** | of R4-H** | of (I) |
| 180 | 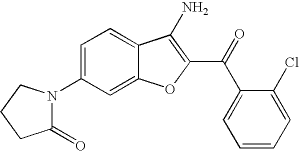 | R_{f} = 0.41 (75% EtOAc/HEX) | 355/357 | A-2 | comm | E-1 |
| 181 | 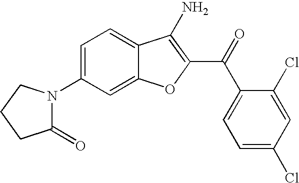 | R_{f} = 0.14 (50% EtOAc/HEX) | 389/391 | comm | comm | E-1 |
| 182 | 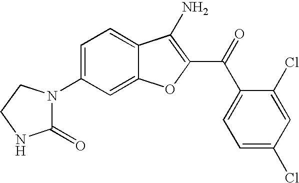 | R_{f} = 0.18 (75% EtOAc/HEX) | 390/392 | comm | comm | E-1 |
| 183 | 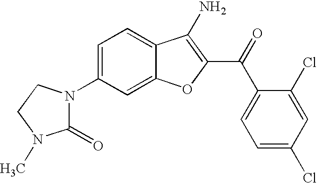 | R_{f} = 0.16 (50% EtOAc/HEX) | 404/406 | comm | comm | E-1 |
| 184 | 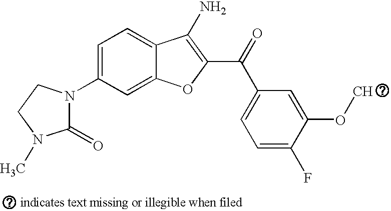 | R_{f} = 0.23 (75% EtOAc/HEX) | 384.0 | A-2 | comm | E-1 |
| 185 | 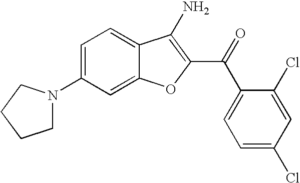 | R_{f} = 0.16 (25% EtOAc/HEX) | 375.0 | comm | comm | E-2 |
| 186 | 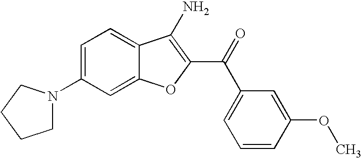 | R_{f} = 0.30 (50% EtOAc/HEX) | 337.0 | comm | comm | E-2 |
| 187 | 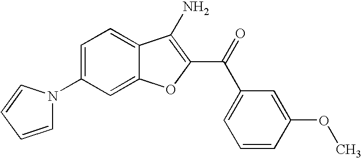 | R_{f} = 0.12 (25% EtOAc/HEX) | 333.0 | comm | comm | E-3 |
| 188 | 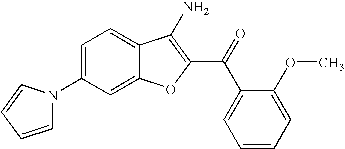 | R_{f} = 0.1 (25% EtOAc/HEX) | 333.0 | comm | comm | E-3 |
| 189 | 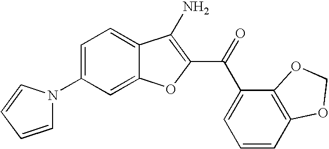 | RT = 3.23 | 347.3 | A-5 | comm | E-3 |
| 190 | 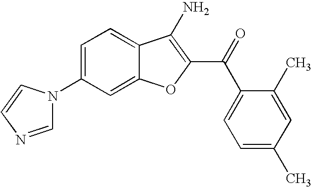 | R_{f} = 0.26 (5% MeOH/EtOAc) | 332.0 | comm | comm | E-4 |
| 191 | 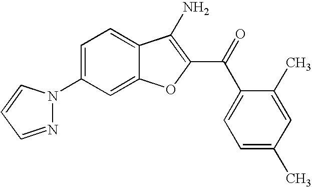 | R_{f} = 0.11 (25% EtOAc/HEX) | 332.0 | comm | comm | E-4 |
| 192 | 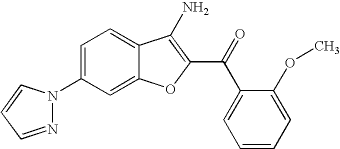 | R_{f} = 0.17 (25% EtOAc/HEX) | 334.0 | comm | comm | E-4 |
| 193 | 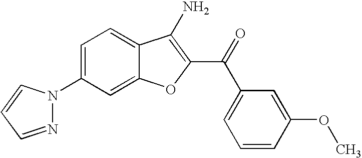 | R_{f} = 0.08 (25% EtOAc/HEX) | 334.0 | comm | comm | E-4 |
| 194 | 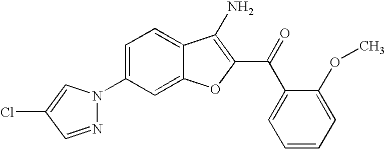 | R_{f} = 0.345 (50% EtOAc/HEX) | 368.0 | comm | comm | E-4 |

Footnotes:
*The following are the LCMS conditions: HPLC-electrospray mass spectra (HPLC ES-MS) were obtained using a Gilson HPLC system equipped with two Gilson 306 pumps, a Gilson 215 Autosampler, a Gilson diode array detector, a YMC Pro C-18 column (2 × 23 mm, 120 A), and a Micromass LCZ single quadrupole mass spectrometer with z-spray electrospray ionization. Spectra were scanned from 120-1000 amu over 2 seconds. ELSD (Evaporative Light Scattering Detector) data was also acquired as an analog channel. Gradient elution was used with Buffer A as 2% acetonitrile in water with 0.02% TFA and Buffer B as 2% water in Acetonitrile with 0.02% TFA at 1.5 mL/min. Samples were eluted as follows: 90% A for 0.5 min ramped to 95% B over 3.5 min and held at 95% B for 0.5 min and then the column is brought back to initial conditions over 0.1 min. Total run time is 4.8 min.
**comm means commercially available.

General Method F: Preparation of Compounds of Formula (I) from Other Compounds of Formula I

The various means used for the further derivatization of compounds of formula I (prepared by means described above) into other compounds of formula (I) are described in the examples below.

### Example 195

### Method F-1a

### Preparation of N-[2-(2,4-Dichloro-benzoyl)-6-phenyl-benzofuran-3-yl]-acetamide

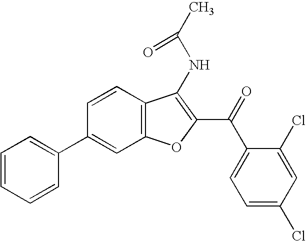

A mixture of (3-amino-6-phenyl-1-benzofuran-2-yl)(2,4-dichlorophenyl)methanone (129 mg, 0.337 mmol), acetyl chloride (0.10 mL, 1.41 mmol, 4.2 eq), diisopropylethylamine polystyrene resin (100 mg, 3.75 mmol/g loading, 1.1 eq) in anhydrous dichloroethane was shaken at 40° C. for 4 days. The reaction mixture was filtered, and the filtrate was concentrated. The crude product was purified on the MPLC (Biotage) eluted with 10% ethyl acetate-hexane. Crystallization from ether-hexane afforded 86.8 mg (60.6%) of the product. ¹H-NMR (DMSO-d₆) δ 10.36 (s, 1H), 8.03 (d, J=8.7 Hz, 1H), 7.95 (d, J=1.0 Hz, 1H), 7.83 (d, J=1.8 Hz, 1H), 7.77 (d, J=6.9 Hz, 1H), 7.72 to 7.62 (m, 4H), 7.49 to 7.39 (m, 3H), 2.12 (s, 3H); LC-MS (ES MH⁺=423, RT=4.01 min).

### Example 196

### Method F-1b

### Preparation of N-[6-(3-Cyano-phenyl)-2-(2,4-dichloro-benzoyl)-benzofuran-3-yl]-acetamide

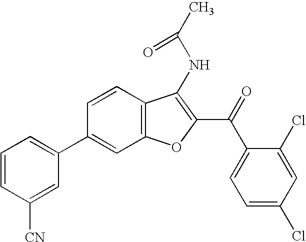

To the solution of 3-[3-amino-2-(2,4-dichloro-benzoyl)-benzofuran-6-yl]-benzonitrile (prepared according to Method C-1)(200 mg, 0.5 mmol) in anhydrous THF (2 mL) was added acetic anhydride (0.12 mL, 1.2 mmol, 2.5 eq) and sodium acetate (100 mg, 1.2 mmol, 2.5 eq). The reaction mixture was stirred at 60° C. for 40 h. While cooling down to rt, some white solid precipitated and was filtered off. The filtrate was evaporated in vacuo and washed with water and EtOAc. Drying with high vacuum pump gave 120 mg (55%) N-[6-(3-Cyano-phenyl)-2-(2,4-dichloro-benzoyl)-benzofuran-3-yl]-acetamide as a yellow solid. ¹H-NMR (CDCl₃) δ 10.36 (broad, s, 1H), 8.67 (d, J=8.8 Hz, 1H), 7.89 to 7.83 (m, 2H), 7.67 to 7.49 (m, 6H), 7.40 (dd, J=8.8 Hz, 2 Hz, 1H), 2.40 (s, 3H). R_{f}=0.62, 50% EtOAc-HEX.

### Example 197

### Method F-2

### Preparation of 3-[3-Amino-2-(2,4-dichloro-benzoyl)-benzofuran-6-yl]-benzamide

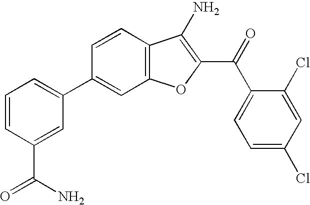

To a solution of 3-amino-6-(3′-cyanophenyl)-1-benzofuran-2-yl)(2,4-dichlorophenyl)methanone (36 mg, 0.09 mmol) in acetone (1.7 mL) and water (0.88 mL) was added sodium percarbonate with 25% hydrogen peroxide (69.4 mg, 0.44 mmol, 5 eq). The reaction mixture was stirred at 60° C. for 7 h. The reaction mixture was cooled and the volatile solvent was evaporated. The residue was diluted with ethyl acetate, washed with water, brine, and dried over sodium sulfate. The solvent was removed at reduced pressure, and the crude product was recrystallized from ethyl acetate-hexane to afford 18.8 mg (50.0%) of the product. ¹H-NMR (DMSO-d₆) δ 8.22 (t, J=1.5 Hz, 1H), 8.14 (d, J=8.7 Hz, 1H), 8.09 (s, 1H), 7.92 to 7.84 (m, 3H), 7.73 (d, J=1.8 Hz, 1H), 7.69 (dd, J=8.1 Hz, 1.5 Hz, 1H), 7.62 to 7.51 (m, 5H), 7.44 (s, 1H); LC-MS (ES MH⁺=425, RT=2.99 min).

### Example 198

### Method F-3

### Preparation of N-{3-[3-amino-2-(2,4-dichloro-benzoyl)-benzofuran-6-yl]-phenyl}-2-methoxyacetamide

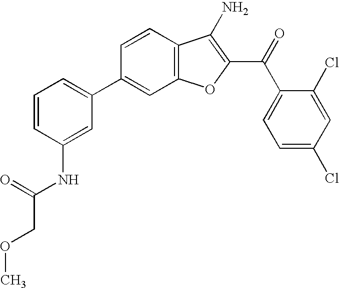

A solution of methoxy acetic acid (27.2 mg, 0.30 mmol, 1.5 eq), 1-(3-dimethylaminopropyl)-3-ethylcarbodiimide hydrochloride (57.9 mg, 0.30 mmol, 1.5 eq), 1-hydroxybenzotriazole hydrate (40.8 mg, 0.30 mmol, 1.5 eq), and diisopropylethylamine (698 mg, 0.60 mmol, 3 eq) in anhydrous 1:1 v/v THF-acetonitrile (5 mL) was stirred at room temperature under argon for 1 h. A solution of [3-amino-6-(3-amino-phenyl)-benzofuran-2-yl]-(2,4-dichlorophenyl)methanone (80 mg, 0.20 mmol) in anhydrous THF (5 mL) was then added, and the reaction mixture was stirred at 80° C. for 18 h. The reaction mixture was diluted with ethyl acetate and water, and the organic layer was washed with water, brine, and dried. The crude product was purified via preparative thin-layer chromatography using 50% ethyl acetate-hexane as the eluant. Crystallization from ether-hexane afforded 28.2 mg (29.9%) of the product. ¹H-NMR (Acetone-d₆) δ 9.10 (broad s, 1H), 8.15 (m, 1H), 8.10 (dd, J=8.1 Hz, 1.8 Hz, 1H), 7.83 (m, 1H), 7.65 to 7.41 (m, 7H), 7.11 (broad, s, 2H), 4.03 (s, 2H), 3.47 (s, 3H); MS ES (MH⁺=496); R_{f}=0.28 (30% ethyl acetate-hexane).

### Example 199

### Method F-4

### Preparation of 2-amino-N-{3-[3-amino-2-(2,4-dichloro-benzoyl)-benzofuran-6-yl]-phenyl}acetamide

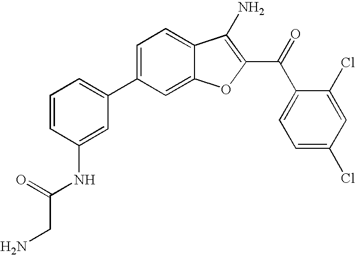

A mixture of N-{3-[3-amino-2-(2,4-dichlorobenzoyl)-benzofuran-6-yl]-phenylcarbamoyl}-methyl)carbamic acid tert-butyl ester prepared according Example 198 F-3 (80 mg, 0.14 mmol) in trifluoroacetic acid (20 mL) and anhydrous THF (40 mL) was stirred under argon at room temperature for 18 h. The reaction mixture was diluted with ethyl acetate and water, and the organic layer was washed with saturated aqueous sodium carbonate, water, brine, and dried over magnesium sulfate. The solvent was evaporated under reduce pressure, and the crude product was purified on the MPLC (Biotage) eluted with 10% dichloromethane-methanol to give 21.3 mg (32.5%) of product. ¹H-NMR (Acetone-d₆) δ 9.96 (broad s, 1H), 8.09 (m, 2H), 8.00 to 7.42 (m, 7H), 7.27 (d, J=7.8 Hz, 1H), 7.10 (broad s, 2H), 4.00 (s, 2H), 2.92 (broad s, 2H); MS ES (MH⁺=454); R_{f}=0.33 (10% dichloromethane-methanol).

### Example 200

### Method F-5

### Preparation of {3-Amino-6-[3-((R)-2,3-dihydroxy-propylamino)-phenyl]-benzofuran-2-yl}-(2,4-dichloro-phenyl)-methanone

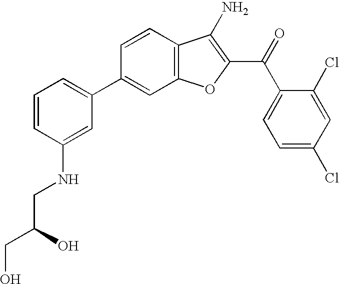

A mixture of 3-amino-6-(3′-aminophenyl)-1-benzofuran-2-yl)(2,4-dichlorophenyl)methanone (150.0 mg, 0.38 mmol) and (S)-(−)-glycidol (0.03 mL, 0.38 mmol, 1.0 eq) in 2:1 v/v dioxane-water was stirred at 80° C. for 16 h. The reaction was diluted with ethyl acetate, washed with water, brine, and dried over sodium sulfate. The solvent was removed at reduced pressure, and the crude material was purified on the MPLC (Biotage) eluted with 5% methanol-ethyl acetate to afford 78.2 mg (43.9%) of the product. ¹H-NMR (Acetone-d₆) δ 8.03 (d, J=8.4 Hz, 1H), 7.65 to 7.53 (m, 5H), 7.20 (t, J=8.1 Hz, 1H), 7.70 (broad s, 2H), 7.04 (t, J=2.1 Hz, 1H), 6.95 (d, J=8.1 Hz, 1H), 6.72 (dd, J=7.8 Hz, 2.4 Hz, 1H), 5.03 (broad s, 1H), 3.96 (broad s, 1H), 3.89 (t, J=5.4 Hz, 1H), 3.74 (broad s, 1H), 3.66 to 3.59 (m, 2H), 3.44 to 3.36 (m, 1H), 3.21 to 3.12 (m, 1H); LC-MS (ES MH⁺=471, RT=2.82 min).

### Example 201

### Method F-6a

### Preparation of (3-Amino-6-piperidin-3-yl-benzofuran-2-yl)-(2,4-dichloro-phenyl)-methanone

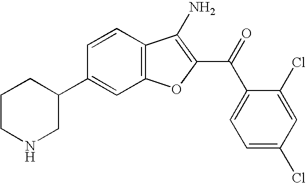

PtO₂ (4.0 mg, 0.018 mmol) was added to a dry flask. Methanol (0.6 mL), tetrahydrofuran (0.5 mL), and hydrogen chloride (50 μl, 2N in dioxane) were added after the flask was flushed with argon. (3-amino-6-pyridin-3-yl-benzofuran-2-yl)-(2,4-dichloro-phenyl)-methanone (40 mg, 0.10 mmol) was added to the flask under argon atmosphere. The solution was degassed under vacuum and refilled with argon. Hydrogen gas was introduced to the flask by a balloon. The mixture was stirred under an H₂ atmosphere at room temperature overnight. The mixture was then filtered and the filtrate concentrated in vacuo. The resulting residue was purified by HPLC to afford 15.5 mg (38.2%) of the title compound. ¹H-NMR (CDOD₃) δ 7.89 (d, J=8.1 Hz, 1H), 7.58 (d, J=1.7 Hz, 1H), 7.47 (m, 2H), 7.29 (s, 1H), 7.23 (dd, J=8.2 Hz, 1.7 Hz, 1H), 3.44 (m, 2H), 3.08 (m, 3H), 2.07 (t, 2H), 1.86 (m, 2H); MS LC-MS (MH⁺=389.6).

### Example 202

### Method F-6b

### Preparation of 1-{3-[3-Amino-2-(2,4-dichloro-benzoyl)-benzofuran-6-yl]-piperidin-1-yl}-3-diethylamino-propan-1-one

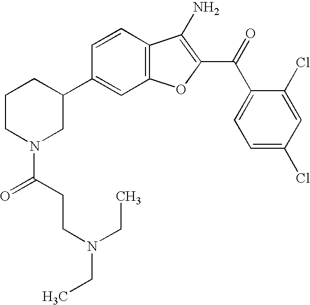

A mixture of (3-amino-6-piperidin-3-yl-benzofuran-2-yl)-(2,4-dichloro-phenyl)-methanone (65 mg, 0.17 mmol), (3-dimethylamino-propyl)-ethyl-carbodiimide hydrochloride (35 mg, 0.18 mmol), and 1-hydroxybenzotriazole (25 mg, 0.18 mmol) in methylene chloride (1.5 mL) was stirred at room temperature for 10 min. Triethylamine (70 μl, 0.50 mmol) and 3-diethylamino-propionic acid (24 mg, 0.17 mmol) were added to the mixture. The solution was stirred at room temperature overnight. The solution was concentrated in vacuo and the resulting residue was purified by HPLC to afford 31 mg (42%) of the title compound. ¹H-NMR (CDCl₃) δ 7.54 (d, J=8.8 Hz, 1H), 7.42 (m, 2H), 7.29 (m, 1H), 7.12 (d, J=4.9 Hz, 1H), 7.06 (d, J=8.4 Hz, 1H), 6.04 (d, 2H), 4.67 (t, J=14.5 Hz, 1H), 3.87 (m, 1H), 2.99 (t, J=12.5 Hz, 1H), 2.76 (m, 3H), 2.49 (m, 6H), 2.00 (m, 1H), 1.79 (m, 1H), 1.62 (m, 1H), 1.54 (m, 1H), 0.96 (m, 6H); MS LC-MS (MH⁺=516.9).

### Example 203

### Method F-6c

### Preparation of [3-Amino-6-(1-isopropyl-piperidin-3-yl)-benzofuran-2-yl]-(2,4-dichloro-phenyl)-methanone

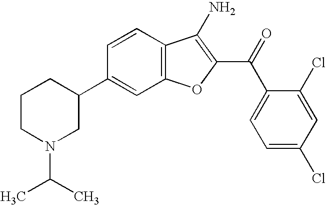

A mixture of (3-amino-6-piperidin-3-yl-benzofuran-2-yl)-(2,4-dichloro-phenyl)-methanone (50 mg, 0.13 mmol), acetone ((10 μl, 0.13 mmol), sodium triacetoxyborohydride (38 mg, 0.18 mmol), triethylamine (27 μl, 0.19 mmol), and acetic acid (7.0 μl, 0.13 mmol) in 1,2-dichloroethane (1.3 mL) was stirred at room temperature overnight. The solution was concentrated. The resulting residue was purified by HPLC to afford 18 mg (32%) of the title compound. ¹H-NMR (CDCl₃) δ 7.53 (d, J=8.4 Hz, 1H), 7.49 (s, 1H), 7.48 (d, J=4.6 Hz, 1H), 7.35 (dd, J=8.7, 2.2 Hz, 1H), 7.21 (s, 1H), 7.15 (d, J=8.7 Hz, 1H), 6.02 (s, 2H), 2.94 (m, 3H), 2.76 (m, 1H), 2.16 (m, 2H), 1.94 (m, 1H), 1.80 (m, 1H), 1.69 (m, 1H), 1.46 (m, 1H), 1.04 (d, J=2.6 Hz, 3H), 1.02 (d, J=2.6 Hz, 3H); MS LC-MS (MH⁺=431.7).

### Example 204

### Method F-6d

### Preparation of [3-Amino-6-(1-butyl-piperidin-3-yl)-benzofuran-2-yl]-(2,4-dichloro-phenyl)-methanone

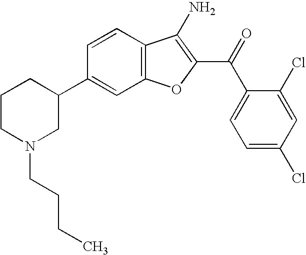

This compound was prepared from (3-amino-6-piperidin-3-yl-benzofuran-2-yl)-(2,4-dichloro-phenyl)-methanone (50 mg, 0.13 mmol) in the manner described for [3-amino-6-(1-isopropyl-piperidin-3-yl)-benzofuran-2-yl]-(2,4-dichloro-phenyl)-methanone affording 27 mg (47%) of the title compound as a yellow solid. ¹H-NMR (CDCl₃) δ 7.53 (d, J=8.3 Hz, 1H), 7.49 (d, J=2.1 Hz, 1H), 7.48 (d, J=4.2 Hz, 1H), 7.34 (dd, J=8.5, 2.1 Hz, 1H), 7.21 (s, 1H), 7.14 (dd, J=8.5, 2.4 Hz, 1H), 6.01 (s, 2H), 2.97 (m, 3H), 2.33 (m, 2H), 1.94 (m, 3H), 1.75 (m, 2H), 1.46 (m, 3H), 1.29 (m, 2H), 0.89 (t, J=7.3 Hz, 3H); MS LC-MS (MH⁺=445.4).

### Example 205

### Method F-7a

### Preparation of 2-{3-[3-Amino-2-(2,4-dichloro-benzoyl)-benzofuran-6-yl]-phenyl}-acetamidine trifluoro-acetic acid

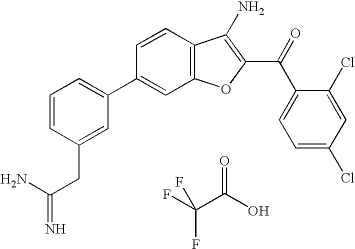

A solution of 3-[3-amino-2-(2,4-dichloro-benzoyl)-benzofuran-6-yl]-benzonitrile (160 mg, 38.0 mmol) in methanol (10 mL) was saturated with hydrochloride gas at 0° C., and stirred at room temperature for 1 h. Then it was saturated with more hydrochloride gas and stirred at room temperature for another 1 h until TLC showed no starting material left. The solvent was removed under reduced pressure. The resulting crude 3-[3-amino-2-(2,4-dichloro-benzoyl)-benzofuran-6-yl]-benzimidic acid methyl ester residue was treated with ammonia in methanol (7 N, 10 mL) and stirred at room temperature overnight. The solvent was removed at reduced pressure. After HPLC separation, a white solid product (34.4 mg, 16.4%) of 2-{3-[3-amino-2-(2,4-dichloro-benzoyl)-benzofuran-6-yl]-phenyl}-acetamidine trifluoro-acetic acid was obtained. ¹H-NMR (CD₃OD) δ 6.34 (d, J=8.7 Hz, 1H), 6.07 (s, 1H), 6.03 (d, J=7.7 Hz, 1H), 5.94 (m, 3H), 5.86 (t, J=8.0 Hz, 2H), 5.82 (s, 1H), 5.77 (t, J=7.4 Hz, 1H), 2.23 (s, 2H); MS LC-MS (MH⁺=438.3), LC MS RT: 2.42 min.

### Example 206

### Method F-7b

### Preparation of 3-[3-Amino-2-(2,4-dichloro-benzoyl)-benzofuran-6-yl]-N,N-dimethyl-benzamidine

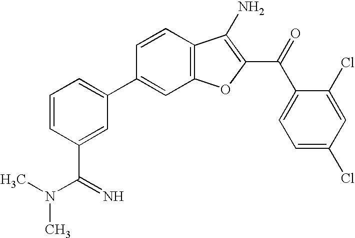

To anhydrous methanol (10 mL) was added 3-[3-amino-2-(2,4-dichloro-benzoyl)-benzofuran-6-yl]-benzonitrile (1.0 g, 2.46 mmol). The solution was then saturated with HCl gas. This was stirred at rt for one h. The solution was then concentrated in vacuo and a portion of the residue (0.070 g, 0.16 mmol) was dissolved in anhydrous MeOH (2 mL) under an inert atmosphere. To this was added dimethylamine (3.1 g, 69 mmol). The solution was then allowed to stir for 72 h at rt. The solution was then concentrated in vacuo, and purified via HPLC to yield 0.025 g (34.7%) of 3-[3-amino-2-(2,4-dichloro-benzoyl)-benzofuran-6-yl]-N,N-dimethyl-benzamidine. ¹HNMR (MeOH-d₄) δ 7.99 (d, J=9.3 Hz, 1H), d 7.78 (d, J=8 Hz 1H), d 7.69 (s, 1H), d 7.62-7.39 (m, 7H), d 2.98 (s, 6H); LC-MS RT: 2.45 min, M+H⁺: 452.3/454.2/456.2.

### Example 207

### Method F-8

### Preparation of [3-Amino-6-(3-methylamino-phenyl)-benzofuran-2-yl]-(2,4-dichloro-phenyl)-methanone

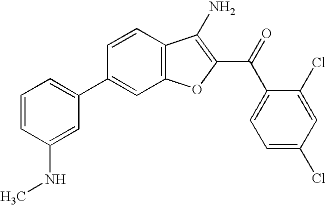

A mixture of [3-amino-6-(3-amino-phenyl)-benzofuran-2-yl]-(2,4-dichloro-phenyl)-methanone (100 mg, 0.25 mmol), formaldehyde (7.5 mL, 0.26 mmol), sodium triacetoxyborohydride (75 mg, 0.35 mmol), and acetic acid (15 μL, 0.25 mmol) in 1,2-dichloroethane was stirred at room temperature overnight. The solvent was removed at reduced pressure and the residue was purified on the MPLC (Biotage) eluted with 5% to 30% ethyl acetate-hexane affording as a yellow solid (13.7 mg, 13.2%). ¹H-NMR (CDCl₃) δ 7.65 (d, J=8.8 Hz, 1H), 7.54 (s, 1H), 7.51 (m, 2H), 7.37 (dd, J=8.2, 2.0 Hz, 1H), 7.27 (t, J=7.8 Hz, 1H), 7.26 (s, 1H), 6.95 (d, J=7.6 Hz, 1H), 6.82 (t, J=2.0 Hz, 1H), 6.75 (dd, J=8.2 Hz, 2.5 Hz, 1H), 6.01 (s, 2H), 2.89 (s, 3H); MS LC-MS (MH⁺=411.2), RT=3.47 min.

### Example 208

### Method F-9

### Preparation of (3-Amino-6-pyridin-3-yl-benzofuran-2-yl)-(2,4-dichlorophenyl)methanone hydrochloride

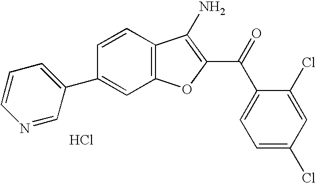

To a solution of (3-Amino-6-pyridin-3-yl-benzofuran-2-yl)-(2,4-dichlorophenyl)methanone (80 mg, 0.21 mmol) in hot ethanol (3 mL) was added concentrated hydrochloric acid (0.16 mL, 5.22 mmol, 25 eq). The reaction mixture was stored at RT for 18 h and at 3° C. for 24 h until crystalline solid is formed. The yellow precipitate was filtered and washed with cold ethanol to afford 37.5 mg (42.8%) of the hydrochloride salt. ¹H-NMR (DMSO-d₆) [symbol] 9.19 (s, 1H), 8.78 (d, J=5.1 Hz, 1H), 8.66 (d, J=7.5 Hz, 1H), 8.22 (d, J=8.4 Hz, 1H), 7.99 (s, 1H), 7.90 (t, J=6.6 Hz, 1H), 7.78 to 7.75 (m, 2H), 7.64 to 7.55 (m, 4H); MS ES (MH⁺=383; retention time=2.48 min).

Other compounds in Table 5 were be prepared in like manner as described above for Examples 195 F1-208 F-9, starting from the appropriate starting materials that are either readily available and/or the synthesis of which is taught herein, and using the process described above or other standard chemical processes known in the art.

TABLE 5
Examples Synthesized using Method F

| | | | | | Synthesis | |
|---|---|---|---|---|---|---|
| Exam- | | R_{f} (TLC solvent) | LC/MS | Synthesis | of (VI) | Synthesis |
| ple | Structure | Or RT (min)* | ([M + H]+) | of (III)** | or (VII)** | of (I) |
| 209 | 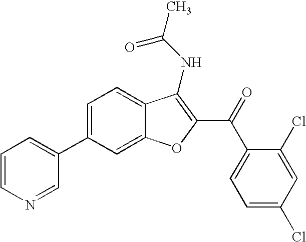 | R_{f} = 0.37 (75% EtOAc/HEX) | 425.0 | comm | comm | C-1, F-1 |
| 210 | 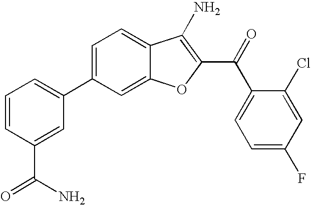 | R_{f} = 0.29 (100% EtOAc/HEX) | 409.0 | A-2 | comm | C-1, F-2 |
| 211 | 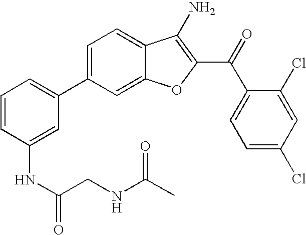 | R_{f} = 0.10, HEX/EtOaC = 50/50 | 496.0 | comm | comm | C1, F-3 |
| 212 | 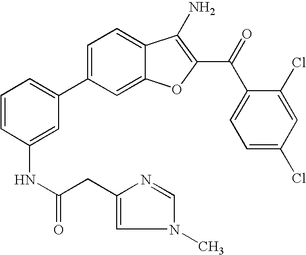 | R_{f} = 0.08, HEX/EtOAc = 50/50, 90% PURE | 519/521 | comm | comm | C1, F-3 |
| 213 | 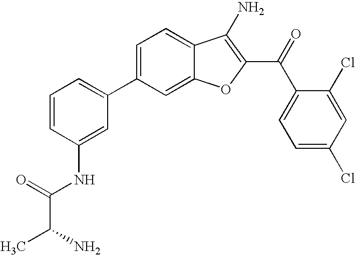 | R_{f} = 0.30, CH₂Cl₂/MeOH = 90/10 | 468.0 | comm | comm | C-1, F-3, F-4 |
| 214 | 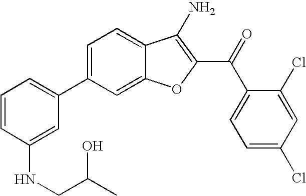 | R_{f} = 0.22 (50% EtOAc/HEX) | 455.0 | comm | comm | C-1, F-5 |
| 215 | 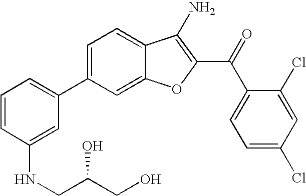 | R_{f} = 0.28 (100% EtOAc) | 471.0 | comm | comm | C-1, F-5 |
| 216 | 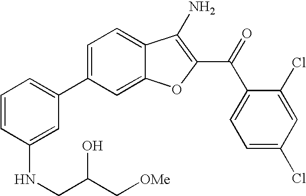 | R_{f} = 0.13 (50% EtOAc/HEX) | 485.0 | comm | comm | C-1, F-5 |
| 217 | 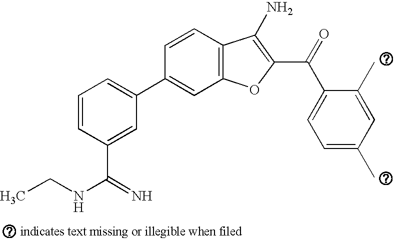 | RT = 2.55 | 452.3 | comm | comm | C-1, F-7 |
| 218 | 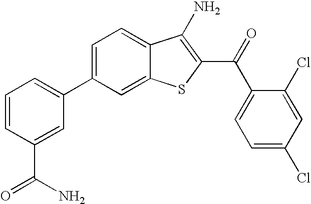 | R_{f} = 0.10, HEX/EtOAc = 50/50 | 441 | comm | comm | G, F-2 |
| 219 | 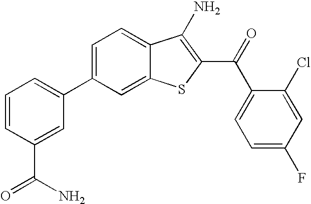 | R_{f} = 0.08, HEX/EtOAc = 50/50 | 425 | A-4 | comm | G, F-2 |
| 220 | 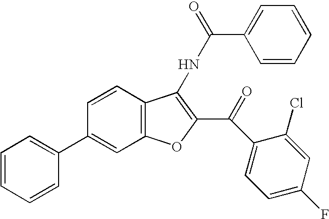 | RF = 0.75 (25% EA/Hex) | [Elemental Analysis Calcd for C₂₈H₁₇Cl₂NO₃: % C 69.15 % H 3.52 % N 2.88, Found: | comm | | B-1, F-1 |

Footnotes:
*The following are the LCMS conditions: HPLC-electrospray mass spectra (HPLC ES-MS) were obtained using a Gilson HPLC system equipped with two Gilson 306 pumps, a Gilson 215 Autosampler, a Gilson diode array detector, a YMC Pro C-18 column (2 × 23 mm, 120 A), and a Micromass LCZ single quadrupole mass spectrometer with z-spray electrospray ionization. Spectra were scanned from 120-1000 amu over 2 seconds. ELSD (Evaporative Light Scattering Detector) data was also acquired as an analog channel. Gradient elution was used with Buffer A as 2% acetonitrile in water with 0.02% TFA and Buffer B as 2% water in Acetonitrile with 0.02% TFA at 1.5 mL/min. Samples were eluted as follows: 90% A for 0.5 min ramped to 95% B over 3.5 min and held at 95% B for 0.5 min and then the column is brought back to initial conditions over 0.1 min. Total run time is 4.8 min.
**comm means commercially available.

General Method G: Preparation of benzothiophenes of Formula (I)

The preparation of benzothiophenes in this invention is illustrated in the General Scheme below and specifically described in preparation of Example 221.

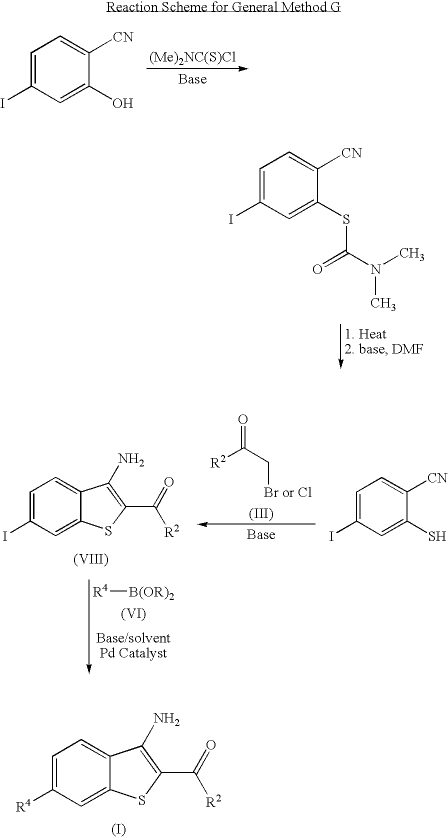

### Example 221

### Method G

### The Preparation of [3-amino-6-(3-pyridinyl)-1-benzothiophene-2-yl](2,4-dichlorophenyl)methanone

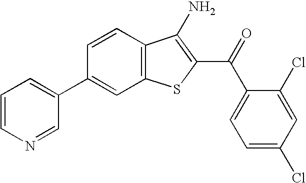

### Step 1: Preparation of 2-cyano-5-iodophenyl-(dimethylamino)methanethioate

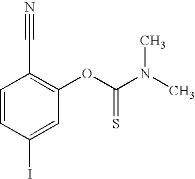

To a solution of 2-cyano-5-iodophenol (10.0 g, 40.8 mmol) in acetone (100 mL) was added dropwise a solution of potassium hydroxide (2.52 g, 44.9 mmol, 1.1 eq) in water (60 mL) at 0° C. After stirring for 45 min, a solution of dimethylthiocarbamoyl chloride (5.55 g, 44.9 mmol, 1.1 eq) in acetone (60 mL) was added at 0° C. over 30 min. The resulted brown reaction mixture was then stirred at room temperature for 16 h, and diluted with ethyl acetate and water. The organic layer was washed with saturated aqueous ammonium chloride, water, and brine. The combined aqueous washes were re-extracted with ethyl acetate, and the organic layers were dried, filtered, and evaporated under reduced pressure. The crude oil was crystallized from ether/hexane to give 2-cyano-5-iodophenyl-(dimethylamino)methanethioate (10.3 g, 76.0%) as a beige solid: ¹H-NMR (Acetone-d₆) δ 7.89 (dd, J=8.4, 1.8 Hz, 1H), 7.79 (d, J=1.5 Hz, 1H), 7.60 (dd, J=8.1 Hz, 1H), 3.44 (s, 6H); MS ES (MH⁺=333); R_{f}=0.70 (30% ethyl acetate-hexane).

### Step 2: Preparation of S-(2-cyano-5-iodo-phenyl)-dimethylthio-carbamic acid

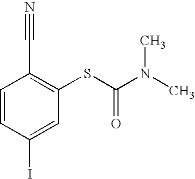

2-Cyano-5-Iodophenyl-(dimethylamino)methanethioate (10.0 g, 30.1 mmol) was heated to a melt at 200° C. under argon for 6 h. The reaction was cooled to room temperature, and the resulted brown solid was purified on the MPLC (Biotage) eluted with 20% ethyl acetate-hexane to give S-(2-cyano-5-iodo-phenyl)-dimethylthio-carbamic acid as a white solid (8.3 g; 83.0%); ¹H-NMR (Acetone-d₆) [symbol] 8.12 (d, J=1.8 Hz, 1H), 8.05 (dd, J=7.8, 1.5 Hz, 1H), 7.66 (d, J=8.1 Hz, 1H), 3.11 (broad s, 3H), 3.08 (broad s, 3H); MS ES (MH⁺=333.0), R_{f}=0.53 (30% ethyl acetate-hexane).

### Step 3: Preparation of 2-cyano-5-iodothiophenol

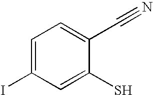

To S-(2-cyano-5-iodo-phenyl)-dimethylthiocarbamic acid (3.0 g, 9.0 mmol) in anhydrous N,N-dimethylformamide (30 mL) under argon was added dropwise 25% sodium methoxide in methanol (6.1 mL, 27.1 mmol, 3.0 eq) at 0° C. The resultant yellow reaction was stirred at room temperature for 1 h. The reaction mixture was poured into cold 2N HCl (100 mL) and then extracted with ethyl acetate. The combined organic layers were dried, filtered, and evaporated under reduced pressure to give crude 2-cyano-5-iodothiophenol. The crude material thus obtained was used directly without further purification.

### Step 4: Preparation of the intermediate 2-[(2′, 4′-dichlorophenyl)carbonyl]-3-amino]-6-iodo benzothiophene

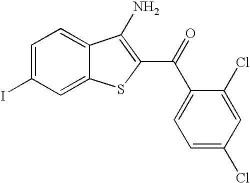

To crude 2-cyano-5-iodothiophenol (3.0 mmol) and 2, 2′,4′-trichloro-acetophenone (673 mg, 3.0 mmol) in anhydrous N,N-dimethylformamide (10 mL) was added powdered potassium hydroxide (832 mg, 6.0 mmol, 2.0 eq). The reaction mixture was stirred under argon at 80° C. for 16 h. The brown reaction mixture was cooled and diluted with ethyl acetate and water. The organic layer was washed with saturated aqueous ammonium chloride, water, brine, and dried. The solvents were evaporated under reduced pressure, and the crude product was purified on the MPLC (Biotage) eluted with 20% ethyl acetate-hexane, followed by trituration from hexane, to give 1.012 g (75.0%) of the benzothiophene. ¹H-NMR (Acetone-d₆) [symbol] 8.24 (d, J=1.5 Hz, 1H), 8.05 (d, J=8.4 Hz, 1H), 7.94 (broad, s, 2H), 7.79 (dd, J=8.4, 1.5 Hz, 1H), 7.65 (dd, J=1.5, 0.9 Hz, 1H), 7.56 (m, 2H); MS ES (MH⁺=448/450); R_{f}=0.58 (30% ethyl acetate-hexane).

### Step 5: Preparation of the title compound: [3-amino-6-(3-pyridinyl)-1-benzothiophene-2-yl](2,4-dichloro-phenyl)methanone

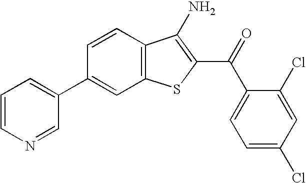

A solution of (3-amino-6-iodo-1-benzothiphene-2-yl)(2,4-dichlorophenyl)methanone (150 mg, 0.33 mmol) in 1,2-dimethoxyethane was degassed with argon for 30 min. At this time, tetrakis(triphenyl phosphine)palladium(0), (39 mg, 0.03 mmol, 0.1 eq) was added, followed by pyridine-3-boronic acid (41 mg, 0.33 mmol, 1.0 eq) and 2M aqueous Na₂CO₃ (4.0 mL). The reaction was bubbled with argon for another 10 min and then heated to 80° C. overnight (18 h). The reaction was diluted with ethyl acetate, washed with water, brine, and dried over magnesium sulfate. The solvent was removed at reduced pressure, and the crude product was purified on the MPLC (Biotage) eluted with 45 to 65% ethyl acetate-hexane to afford 37.5 mg (28.1%) of a yellow solid as the product. ¹H-NMR (Acetone-d₆) [symbol] 8.84 (dd, J=2.7, 0.6 Hz, 1H), 8.49 (dd, J=4.8, 1.8 Hz, 1H), 8.21 (dd, J=8.4, 0.6 Hz, 1H), 8.00 (m, 2H), 7.87 (broad, s, 2H), 7.68 (dd, J=8.4, 1.5 Hz, 1H), 7.53 (d, J=1.8 Hz, 1H), 7.46 (s, 1H); 7.44 (d, J=1.8 Hz, 1H), 7.36 (m, 1H) LC-MS (ES MH⁺=399, RT=2.71 min). R_{f}=0.08 (50% ethyl acetate-hexane).

Other compounds, appearing in Table 6 were prepared in like manner as described above by choosing the appropriate starting materials that are readily available and/or the synthesis of which is taught herein, and using the process described above or other standard chemical processes known in the art.

TABLE 6
Examples Synthesized using Method G

| | | | | | Synthesis | |
|---|---|---|---|---|---|---|
| Exam- | | R_{f} (TLC solvent) | LC/MS | Synthesis | of (VI) | Synthesis |
| ple | Structure | Or RT (min)* | ([M + H]+) | of (III)** | or (VII)** | of (I) |
| 222 | 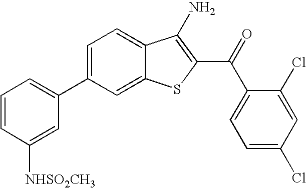 | R_{f} = 0.08, HEX/EtOAc = 50/50 | 491/493 | comm | comm | G |
| 223 | 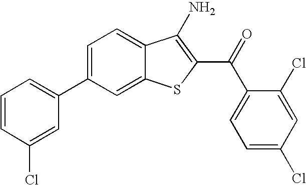 | R_{f} = 0.58, HEX/EtOAc = 70/30 | 434 | comm | comm | G |
| 224 | 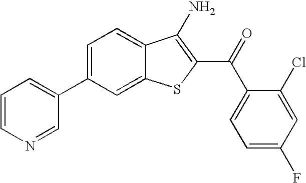 | R_{f} = 0.08, HEX/EtOAc = 50/50 | 383 | A-4 | comm | G |
| 225 | 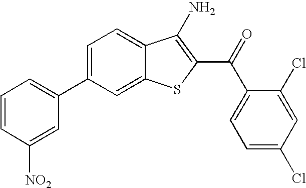 | R_{f} = 0.45, HEX/EtOAc = 70/30 | 443/445 | comm | comm | G |
| 226 | 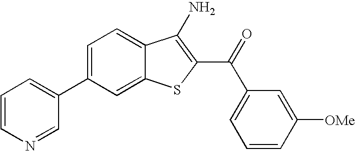 | R_{f} = 0.08, HEX/EtOAc = 60/40 | 361 | comm | comm | G |
| 227 | 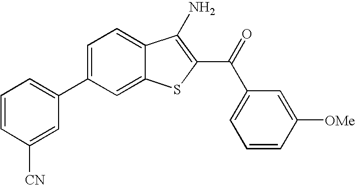 | R_{f} = 0.32, HEX/EtOAc = 70/30 | 385 | comm | comm | G |
| 228 | 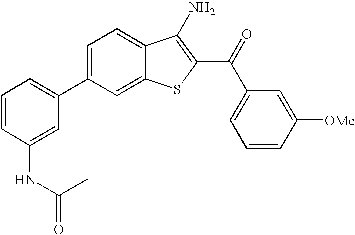 | R_{f} = 0.12, HEX/EtOAc = 60/40 | 417 | comm | comm | G |
| 229 | 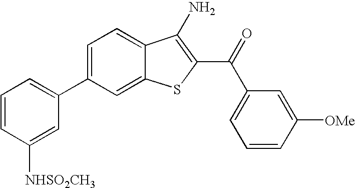 | R_{f} = 0.14, HEX/EtOAc = 60/40 | 453 | comm | comm | G |
| 230 | 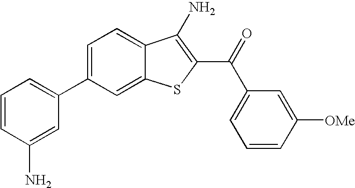 | R_{f} = 0.10, HEX/EtOAc = 70/30 | 375 | comm | comm | G |

Footnotes:
*The following are the LCMS conditions: HPLC-electrospray mass spectra (HPLC ES-MS) were obtained using a Gilson HPLC system equipped with two Gilson 306 pumps, a Gilson 215 Autosampler, a Gilson diode array detector, a YMC Pro C-18 column (2 × 23 mm, 120 A), and a Micromass LCZ single quadrupole mass spectrometer with z-spray electrospray ionization. Spectra were scanned from 120-1000 amu over 2 seconds. ELSD (Evaporative Light Scattering Detector) data was also acquired as an analog channel. Gradient elution was used with Buffer A as 2% acetonitrile in water with 0.02% TFA and Buffer B as 2% water in Acetonitrile with 0.02% TFA at 1.5 mL/min. Samples were eluted as follows: 90% A for 0.5 min ramped to 95% B over 3.5 min and held at 95% B for 0.5 min and then the column is brought back to initial conditions over 0.1 min. Total run time is 4.8 min.
**comm means commercially available.

Other compounds of Formula I may be prepared using the methods described herein or other methods known in the art, and using the appropriate starting materials and/or intermediates that would be readily recognized by those skilled in the art.

TABLE 7

| | |
|---|---|
| | (I) |
| 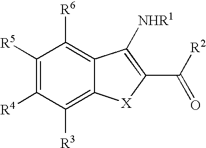 | |

| Example | X | R¹ | R² | R³ | R⁴ | R⁵ | R⁶ |
|---|---|---|---|---|---|---|---|
| 231 | O | PhCO— | 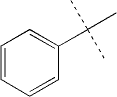 | H | 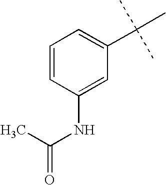 | H | H |
| 232 | O | H | 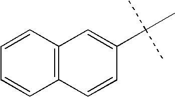 | H | 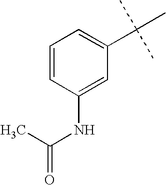 | H | H |
| 233 | S | H | 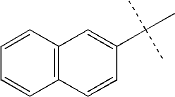 | H | 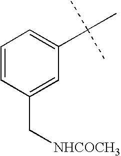 | H | H |
| 234 | O | H | 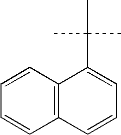 | H | 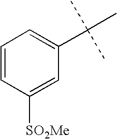 | H | H |
| 235 | O | H | 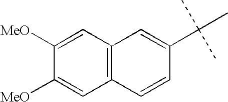 | H | 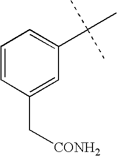 | H | H |
| 236 | O | H | 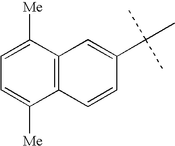 | H | 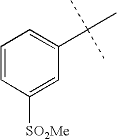 | H | H |
| 237 | S | H | 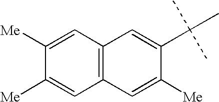 | H | 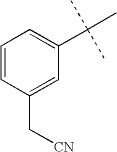 | H | H |
| 238 | O | H | 4-Cl-Ph- | H | 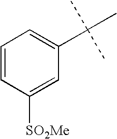 | H | H |
| 239 | O | H | 3-NO₂Ph- | H | 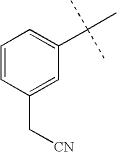 | H | H |
| 240 | S | H | 4-CN-Ph- | H | 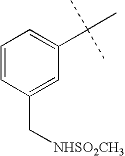 | H | H |
| 241 | S | H | 2,4,6-triCl-Ph- | H | 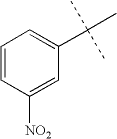 | H | H |
| 242 | O | H | 3,4,5-triMe-Ph | H | 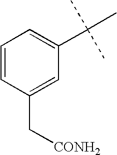 | OH | H |
| 243 | O | H | 4-CF₃-Ph- | H | 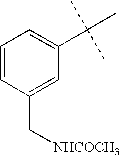 | H | H |
| 244 | O | H | 3-CH₃CO-Ph | H | 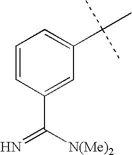 | H | H |
| 245 | O | H | 4-(COOH)-Ph- | H | 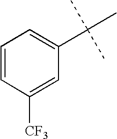 | H | H |
| 246 | O | Et | 3-(CO₂Et)-Ph- | H | 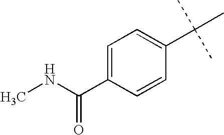 | H | H |
| 247 | O | H | 4-[CON(Me)₂]-Ph | H | 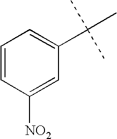 | H | H |
| 248 | O | H | 3-(NHCH₂CH₂SO₂Me)-Ph | H | 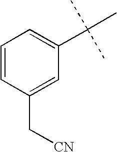 | H | H |
| 249 | O | Me | 4-(NHSO₂Me)-Ph | H | 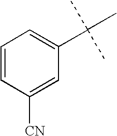 | H | H |
| 250 | O | H | 3-(NHCOEt)-Ph | H | 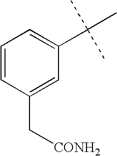 | H | H |
| 251 | O | H | 4-(NH—(CH₂)₄—COMe)-Ph | H | 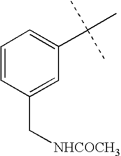 | OMe | H |
| 252 | S | H | 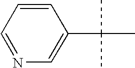 | H | 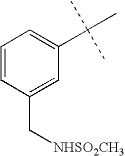 | H | H |
| 253 | S | H | 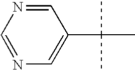 | H | 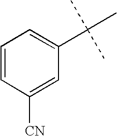 | H | H |
| 254 | O | H | 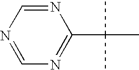 | H | 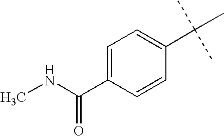 | H | H |
| 255 | O | H | 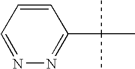 | H | 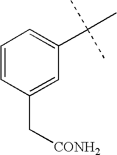 | H | H |
| 256 | O | Ac | 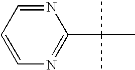 | H | 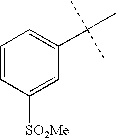 | H | H |
| 257 | O | H | 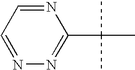 | H | 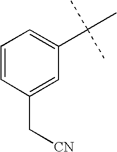 | Me | H |
| 258 | O | H | 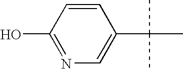 | H | 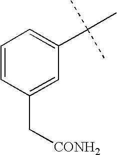 | H | H |
| 259 | O | H | 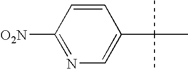 | H | 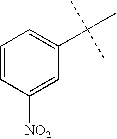 | H | H |
| 260 | O | H | 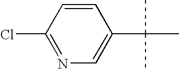 | H | 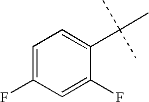 | H | H |
| 261 | S | H | 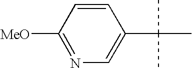 | H | 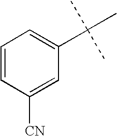 | Cl | H |
| 262 | S | H | 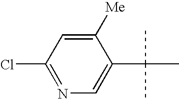 | H | 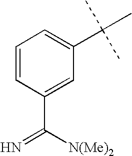 | H | H |
| 263 | O | H | 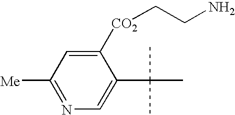 | H | 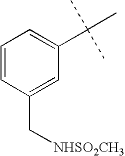 | H | OH |
| 264 | O | Me | 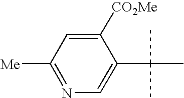 | H | 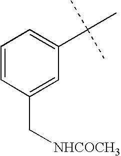 | H | H |
| 265 | O | H | 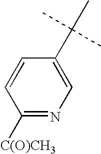 | H | 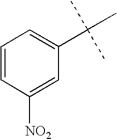 | Me | H |
| 266 | O | H | 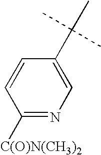 | H | 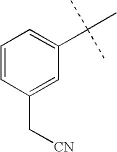 | Cl | H |
| 267 | O | Et | 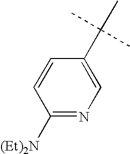 | H | 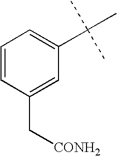 | CF₃ | H |
| 268 | O | H | 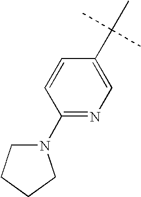 | H | 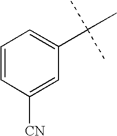 | OH | H |
| 269 | O | H | 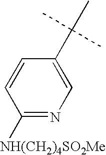 | H | 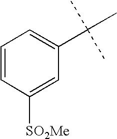 | F | H |
| 270 | S | Et | 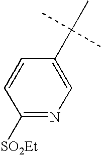 | H | 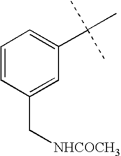 | EtO | H |
| 271 | O | Et | 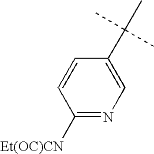 | H | 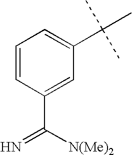 | Cl | H |
| 272 | O | Me | 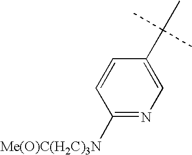 | H | 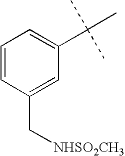 | CF₃O | H |
| 273 | O | H | 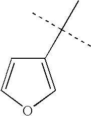 | H | 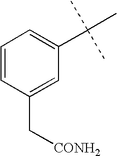 | CH₃ | H |
| 274 | O | H | 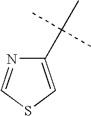 | H | 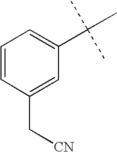 | H | H |
| 275 | O | H | 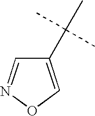 | H | 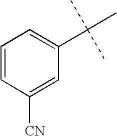 | H | H |
| 276 | S | H | 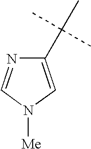 | H | 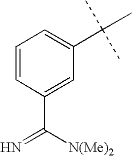 | H | H |
| 277 | S | H | 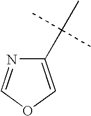 | H | 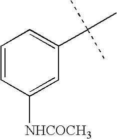 | H | H |
| 278 | O | H | 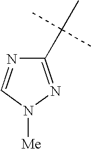 | H | 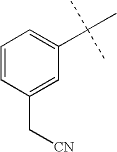 | H | H |
| 279 | S | H | 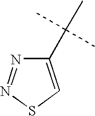 | H | 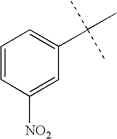 | H | |
| 280 | S | H | 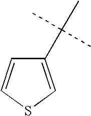 | H | 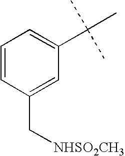 | Cl | H |
| 281 | O | H | 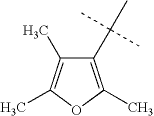 | H | 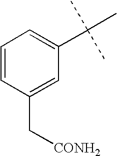 | F | H |
| 282 | O | H | 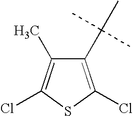 | H | 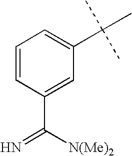 | Me | H |
| 283 | O | H | 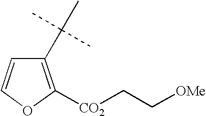 | H | 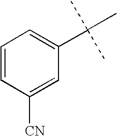 | H | OMe |
| 284 | S | Me | 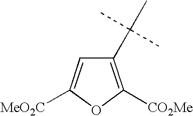 | H | 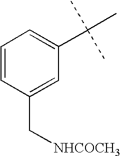 | OH | H |
| 285 | O | H | 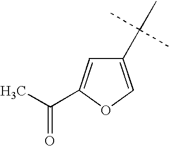 | H | 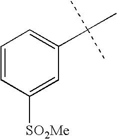 | H | H |
| 286 | O | H | 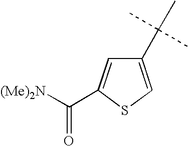 | H | 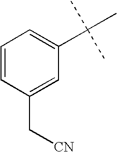 | H | H |
| 287 | O | Et | 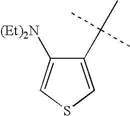 | H | 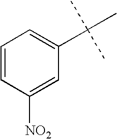 | H | H |
| 288 | O | H | 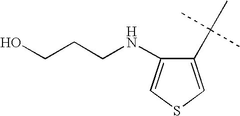 | H | 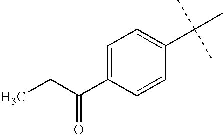 | OH | H |
| 289 | O | Me | 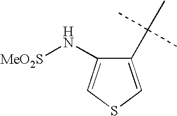 | H | 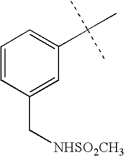 | H | H |
| 290 | O | H | 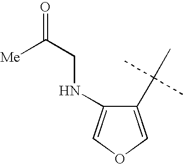 | H | 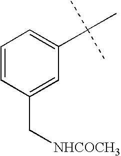 | H | H |
| 291 | O | H | 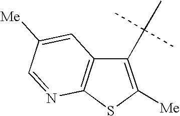 | H | 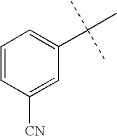 | H | Cl |
| 292 | S | H | 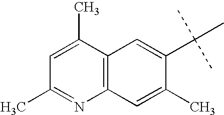 | H | 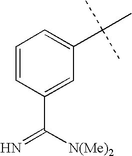 | H | H |
| 293 | S | H | 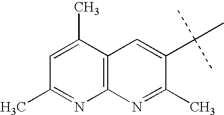 | H | 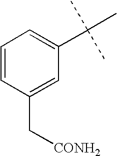 | H | H |
| 294 | O | H | 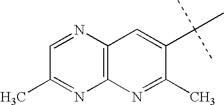 | H | 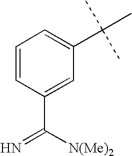 | H | CF₃ |
| 295 | O | H | 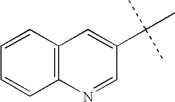 | H | 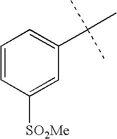 | OMe | H |
| 296 | O | H | 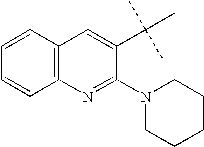 | H | 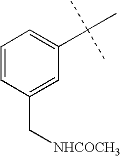 | OH | H |
| 297 | O | Et | 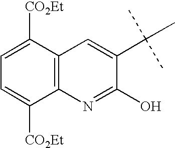 | H | 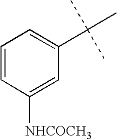 | Me | H |
| 298 | O | H | 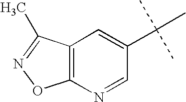 | H | 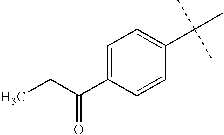 | Cl | H |
| 299 | O | H | 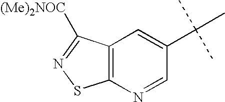 | H | 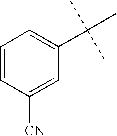 | F | H |
| 300 | O | H | 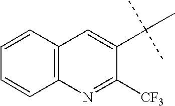 | H | 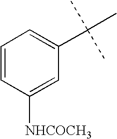 | Et | H |
| 301 | S | H | 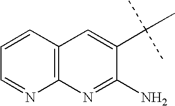 | H | 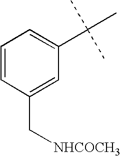 | CF₃O | H |
| 302 | S | Me | 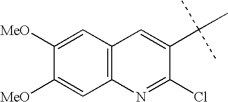 | H | 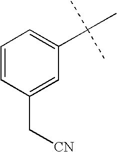 | H | H |
| 303 | O | H | 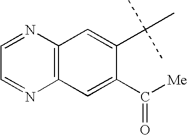 | H | 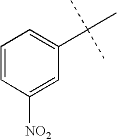 | H | H |
| 304 | O | H | 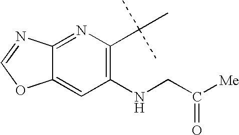 | H | 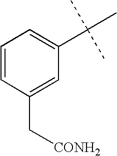 | Me | H |
| 305 | O | H | 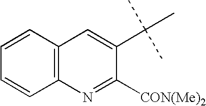 | H | 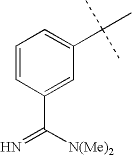 | OH | H |
| 306 | O | H | 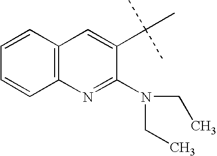 | H | 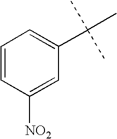 | H | H |
| 307 | O | H | 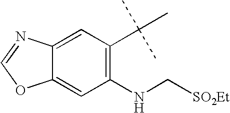 | H | 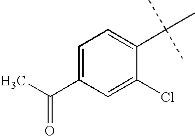 | H | H |
| 308 | O | H | 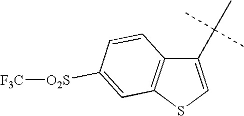 | H | 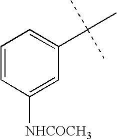 | H | OH |
| 309 | O | H | 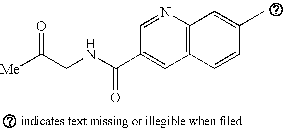 | H | 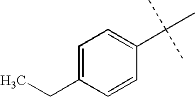 | H | H |
| 310 | O | H | 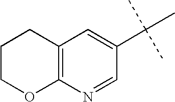 | H | 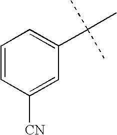 | H | H |
| 311 | O | H | 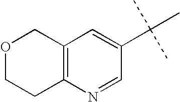 | H | 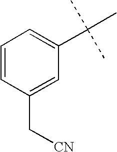 | OH | H |
| 312 | S | H | 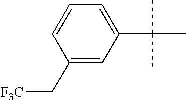 | Cl | 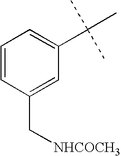 | H | F |
| 313 | O | H | 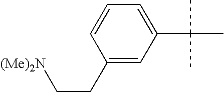 | Me | 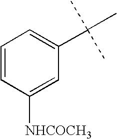 | H | Cl |
| 314 | O | H | 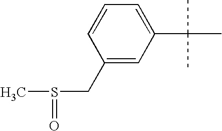 | CF₃ | 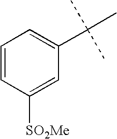 | H | H |
| 315 | O | H | 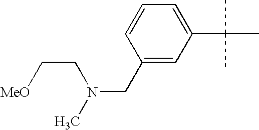 | CF₃O | 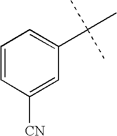 | OMe | H |
| 316 | O | H | 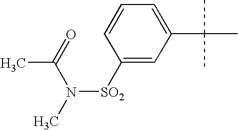 | OH | 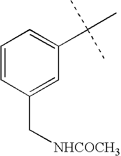 | H | OMe |
| 317 | O | H | 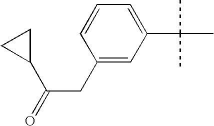 | OH | 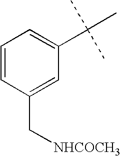 | H | Me |
| 318 | O | H | 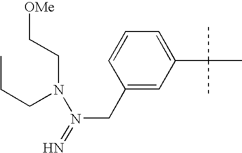 | OMe | 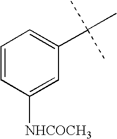 | H | H |
| 319 | O | H | 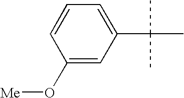 | H | 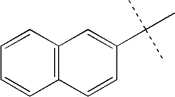 | H | H |
| 320 | O | H | 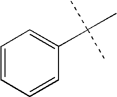 | H | 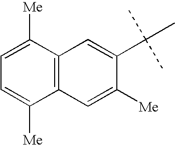 | H | H |
| 321 | O | H | 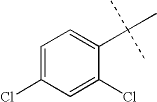 | H | 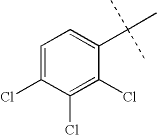 | H | H |
| 322 | O | H | 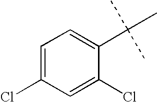 | H | 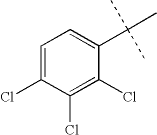 | H | H |
| 323 | O | H | 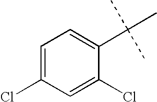 | H | 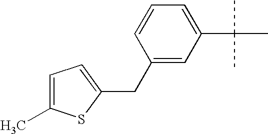 | Me | H |
| 324 | O | H | 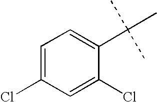 | H | 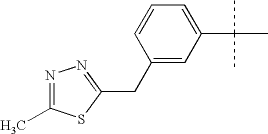 | H | H |
| 325 | O | H | 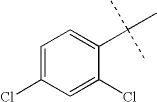 | H | 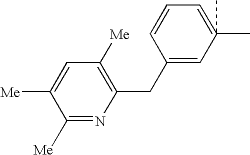 | H | H |
| 326 | O | H | 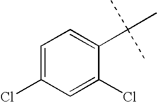 | H | 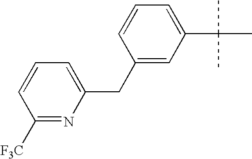 | H | H |
| 327 | O | H | 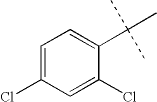 | H | 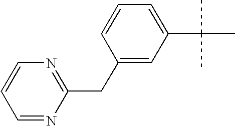 | H | Cl |
| 328 | O | H | 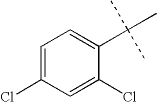 | H | 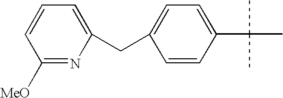 | H | H |
| 329 | O | H | 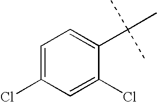 | H | 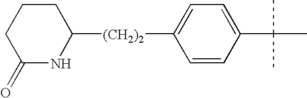 | H | H |
| 330 | O | H | 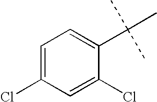 | H | 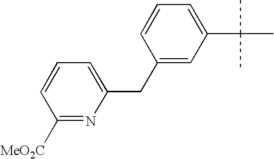 | H | H |
| 331 | O | H | 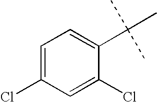 | H | 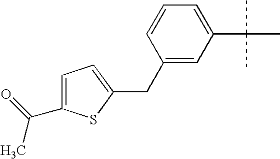 | H | OH |
| 332 | O | H | 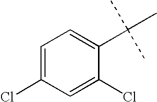 | H | 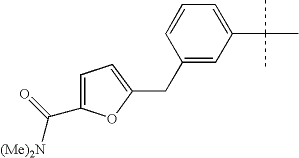 | Me | H |
| 333 | O | H | 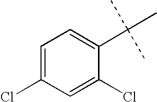 | H | 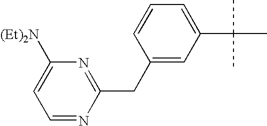 | H | H |
| 334 | O | H | 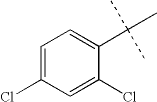 | H | 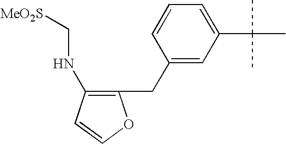 | H | H |
| 335 | O | H | 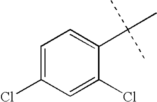 | H | 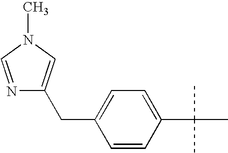 | H | MeO |
| 336 | O | H | 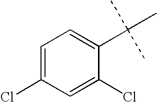 | H | 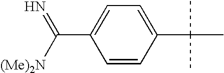 | H | H |
| 337 | O | H | 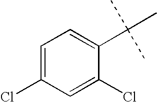 | H | 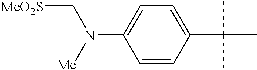 | MeO | H |
| 338 | O | H | 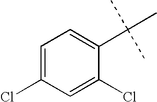 | H | 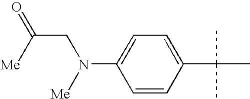 | H | H |
| 339 | O | H | 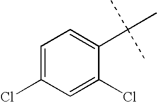 | H | 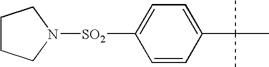 | H | H |
| 340 | O | H | 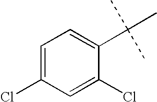 | H | 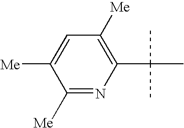 | H | Cl |
| 341 | O | H | 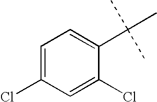 | H | 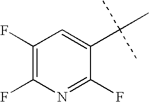 | H | H |
| 342 | O | H | 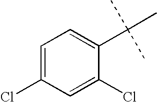 | H | 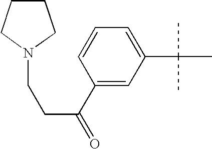 | H | H |
| 343 | O | H | 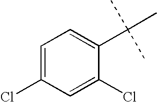 | H | 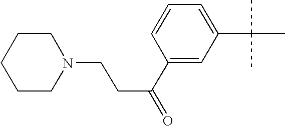 | OH | H |
| 344 | O | H | 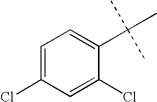 | H | 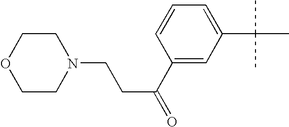 | H | H |
| 345 | O | H | 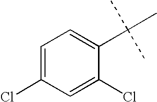 | H | 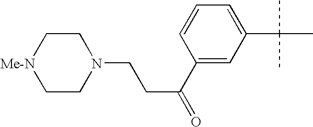 | H | H |
| 346 | O | H | 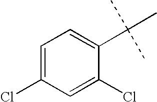 | H | 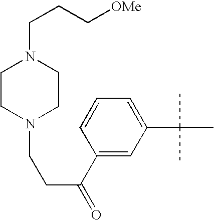 | H | H |
| 347 | O | H | 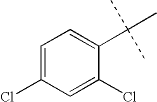 | H | 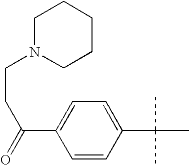 | H | H |
| 348 | O | H | 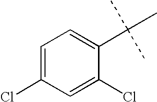 | OH | 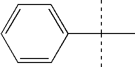 | H | H |
| 349 | O | H | 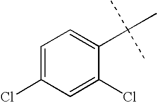 | H | 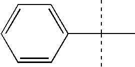 | H | OH |
| 350 | O | H | 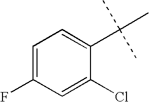 | OH | 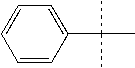 | H | H |
| 351 | O | H | 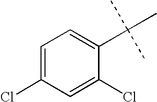 | OH | 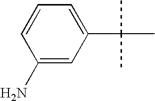 | H | H |
| 352 | O | H | 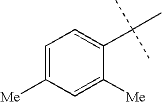 | H | 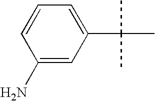 | OH | H |
| 353 | S | H | 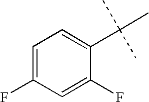 | H | 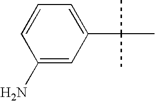 | H | OH |
| 354 | O | H | 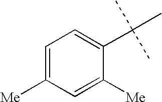 | OH | 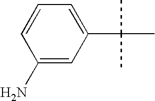 | OH | H |
| 355 | O | H | 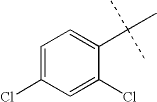 | F | 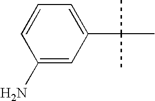 | H | H |
| 356 | O | H | 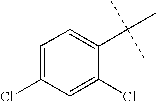 | H | 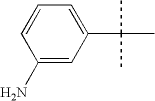 | H | F |
| 357 | O | H | 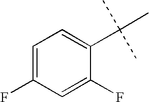 | F | 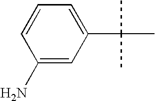 | H | F |
| 358 | O | H | 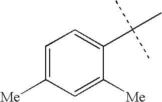 | H | 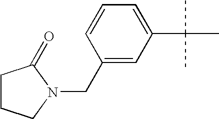 | H | H |
| 359 | O | H | 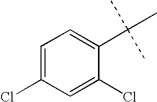 | H | 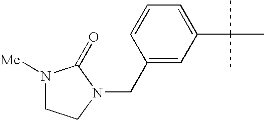 | H | H |
| 360 | O | H | 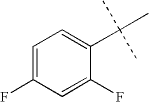 | H | 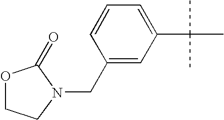 | H | H |
| 361 | S | H | 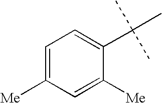 | H | 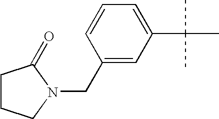 | H | H |
| 362 | O | H | 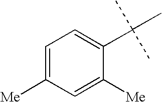 | OH | 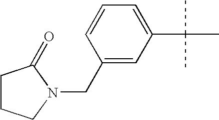 | H | H |
| 363 | O | H | 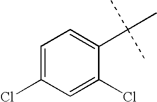 | H | 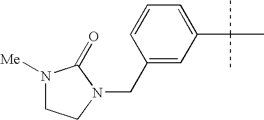 | OH | H |
| 364 | O | H | 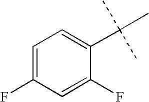 | H | 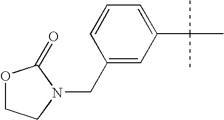 | OH | H |
| 365 | S | H | 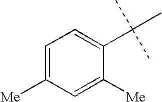 | H | 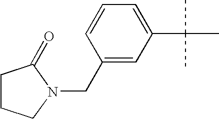 | H | OH |

### Example 366

### N-{3-[3-amino-2-(2,4-dichlorobenzoyl)-1-benzofuran-6-yl]benzyl}-N-(methylsulfonyl)-L-valinamide, hydrochloride

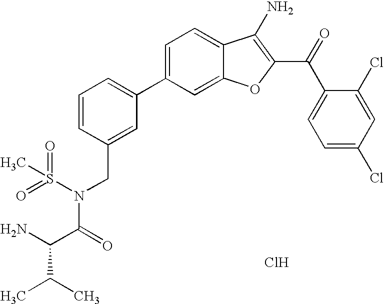

### Acylation:

A solution of 5 g (10.2 mmol) N-{3-[3-amino-2-(2,4-dichlorobenzoyl)-1-benzofuran-6-yl]benzyl}methanesulfonamide in 400 ml of absolute dichloromethane is treated under stirring with 4.97 g (2 equiv.) of tert-butyl (4S)-4-isopropyl-2,5-dioxo-1,3-oxazolidine-3-carboxylate and 2.5 g (2 equiv.) of 4-(N,N-dimethylamino)-pyridine (DMAP). After heating under reflux for 2 hours another 4.97 g (2 equiv.) of tert-butyl (4S)-4-isopropyl-2,5-dioxo-1,3-oxazolidine-3-carboxylate and 2.5 g (2 equiv.) of 4-(N,N-dimethylamino)-pyridine are added and the mixture is refluxed for 16 hours. The mixture is filtrated over silica gel (Amicon SI 60A 35-70 μM) and subsequently the silica gel is washed with 1 l of a mixture of dichloromethane and ethyl acetate. The filtrate is concentrated in vacuo and the remaining residue is purified by flash chromatography on silica gel using dichloromethane/methanol 99.25/0.75 vv as eluent. The fractions of interest are collected and concentrated in vacuo.

6640 mg (94%) of the Boc-protected intermediate are obtained which may contain certain amounts of the D-valine enantiomer, depending on the enantiomeric ratio of the N-carboxy anhydrides employed. The enantiomeric ratio is determined by chiral HPLC (method C1):

t1: 8.00 min, t2: 6.79 min, Ratio L-Val Enantiomer:D-Val Enantiomer: 96.5:3.5

### Deprotection

6640 mg (9.642 mmol) of the Boc-protected intermediate are dissolved in 80 ml of a 13.5% solution of HCl in dioxane and then stirred for ½ hour at RT. After concentrating in vacuo to a small volume, the product is precipitated with diethyl ether, filtrated and thoroughly washed with diethyl ether. 5.89 g (97%) of the target compound are obtained after drying.

TLC: acetonitrile/water (20:1); Rf=0.53, HPLC (method 2): Rt=5.17 min; LC-MS (method 3): Rt=5.82 min; m/z=588 (M+H)+

### Example 367

### N-{3-[3-amino-2-(2,4-dichlorobenzoyl)-1-benzofuran-6-yl]benzyl}-N-(methylsulfonyl)-L-isoleucinamide, hydrochloride

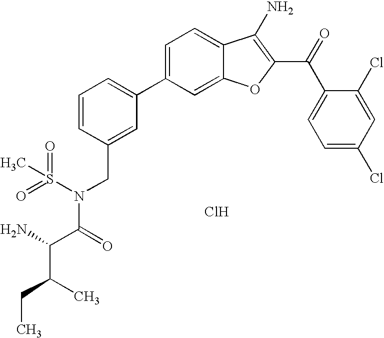

### Acylation:

100 mg (0.2 mmol) N-{3-[3-amino-2-(2,4-dichlorobenzoyl)-1-benzofuran-6-yl]benzyl} methane-sulfonamide is reacted with 526 mg (2 mmol) tert-butyl 4-[(1S)-1-methylpropyl]-2,5-dioxo-1,3-oxazolidine-3-carboxylate in the presence of 250 mg (2 mmol) DMAP as described in example 1. Instead of flash-chromatography preparative HPLC (method 1) is employed to purify the crude product.

Yield of the protected intermediate: 133 mg (93%)

Separation of diastereoisomers by chiral HPLC; method B1, t1: 5.41 min, t2: 7.4 min

Diastereomeric ratio: 90:10 (L-Ile:D-allo-Ile)

The diastereoisomers are separated by chiral HPLC in preparative scale (method B2).

### Cleavage of the Boc-Protecting Group to Give the Title Compound (L-Ile-Conjugate):

The Boc-protecting group is cleaved from the separated Boc-protected intermediate (L-Ile diastereoisomer) with a 13.5% solution of HCl in dioxane as described in example 1.

Yield of the title compound: 6.6 mg

TLC: dichloromethane/methanol/ammonia17% 15/1/0.1; Rf=0.46

HPLC (method 2): Rt=5.24 min;

LC-MS (method 5): Rt=2.0 min; m/z=602 (M+H)+.

### Example 368

### N-{3-[3-amino-2-(2,4-dichlorobenzoyl)-1-benzofuran-6-yl]benzyl}-N-(methylsulfonyl)-D-alloisoleucinamide, hydrochloride

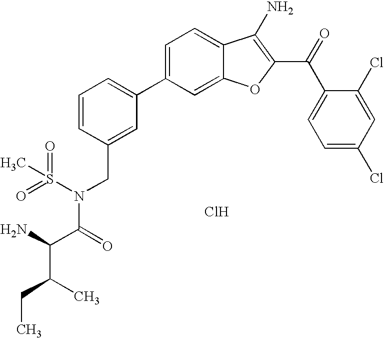

### Cleavage of the Boc-Protecting Group to Give the Title Compound (D-allo-Ile-Conjugate):

The Boc-protecting group is cleaved from the separated Boc-protected intermediate in example 2 (D-allo-Ile diastereoisomer) with a 13.5% solution of HCl in dioxane as described in example 1.

Yield of the title compound: 4 mg

TLC: dichloromethane/methanol/ammonia17% 15/1/0.1; R_{f}=0.4

HPLC (method 2): Rt=5.24 min;

LC-MS (method 5): Rt=2.02 min; m/z=602 (M+H)+

### Example 369

### N-{3-[3-amino-2-(2,4-dichlorobenzoyl)-1-benzofuran-6-yl]benzyl}-O-benzyl-N-(methylsulfonyl)-L-serinamide, hydrochloride

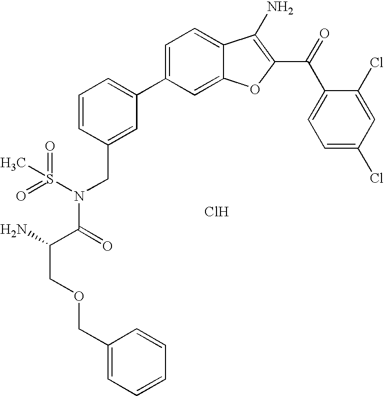

### Acylation:

100 mg (0.2 mmol) N-{3-[3-amino-2-(2,4-dichlorobenzoyl)-1-benzofuran-6-yl]benzyl} methane-sulfonamide is reacted with 657 mg (2 mmol) tert-butyl (4S)-4-[(benzyloxy)methyl]-2,5-dioxo-1,3-oxazolidine-3-carboxylate in the presence of 250 mg (2 mmol) DMAP as described in example 1. Instead of flash-chromatography preparative HPLC (method 1) is employed to purify the crude product.

Yield of the protected intermediate: 78 mg (50%)

Separation of enantiomers by chiral HPLC; method B1, t1: 7.57 min, t2: 9.81 min

Enantiomeric ratio determined by chiral HPLC; method B1: 97:3 (L-Ser:D-Ser)

### Cleavage of the Boc-Protecting Group to Give the Title Compound (L-Ser-(Bzl)-Conjugate):

From 30 mg of the intermediate the Boc-protecting group is cleaved with a 13.5% solution of HCl in dioxane as described in example 1.

Yield of the title compound: 28 mg (quant)

TLC: dichloromethane/methanol/ammonia17% 15/1/0.1; Rf=0.6

HPLC (method 2): Rt=5.29 min;

LC-MS (method 5): Rt=2.09 min; m/z=666 (M+H)+

### Example 370

### Benzyl N-{3-[3-amino-2-(2,4-dichlorobenzoyl)-1-benzofuran-6-yl]benzyl}-N-(methyl sulfon-yl)-L-alpha-asparaginate trifluoroactetate

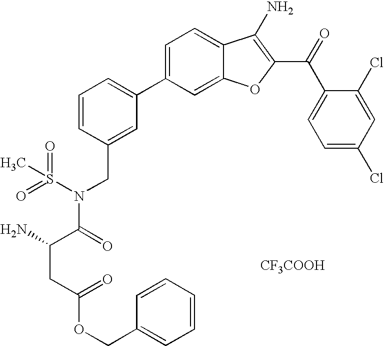

### Acylation:

100 mg (0.2 mmol) N-{3-[3-amino-2-(2,4-dichlorobenzoyl)-1-benzofuran-6-yl]benzyl}methane-sulfonamide is reacted with 714 mg (2 mmol) tert-butyl 4-[2-(benzyloxy)-2-oxoethyl]-2,5-dioxo-1,3-oxazolidine-3-carboxylate in the presence of 250 mg (2 mmol) DMAP as described in example 1. Instead of flash-chromatography preparative HPLC (method 1) is employed to purify the crude product.

Yield of the protected intermediate as an epimeric mixture: 73 mg (45%)

Separation of enantiomers by chiral HPLC; method A1, t1: 3.94 min, t2: 7.12 min

Enantiomeric ratio determined by chiral HPLC; method A1: 53:47 (L-Asp:D-Asp)

The enantiomers are separated by chiral HPLC (method A2).

### Cleavage of the Boc-Protecting Group to Give the Title Compound (L-Asp(Bzl)-Conjugate):

The Boc-protecting group is cleaved from 2 mg of the separated Boc-protected intermediate compound (L-Asp enantiomer) with a trifluoroacetic acid in dichloromethane.

Yield of the title compound: 1.7 mg (85%).

HPLC (method 2): Rt=5.31 min;

LC-MS (method 4): Rt=2.29 min; m/z=694 (M+H)+

### Example 371

### N-{3-[3-amino-2-(2,4-dichlorobenzoyl)-1-benzofuran-6-yl]benzyl}-N-(methylsulfonyl)-D-valinamide, hydrochloride

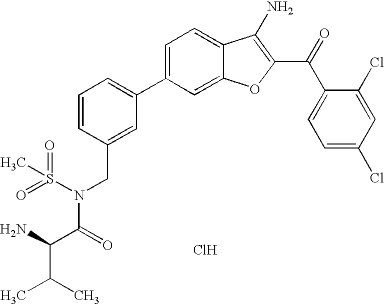

### Acylation:

116 mg (0.24 mmol) N-{3-[3-amino-2-(2,4-dichlorobenzoyl)-1-benzofuran-6-yl]benzyl}methane-sulfonamide is reacted with 243 mg (0.95 mmol) tert-butyl 4-isopropyl-2,5-dioxo-1,3-oxazolidine-3-carboxylate in the presence of 116 mg (0.95 mmol) DMAP as described in example 1. Subsequently to purification by flash-chromatography a second purification step by preparative HPLC (method 1) is employed.

Yield of the protected intermediate: 110 mg (68%)

Separation of enantiomers by chiral HPLC; method Cl, t1: 6.79 min, t2: 8.0 min

Enantiomeric ratio: 97:3 (D-Val:L-Val)

### Cleavage of the Boc-Protecting Group to Give the Title Compound (D-Val-Conjugate):

From 17 mg of the intermediate the Boc-protecting group is cleaved with a 13.5% solution of HCl in dioxane as described in example 1.

Yield of the title compound: 6.5 mg (42%)

HPLC (method 2): Rt=5.19 min;

LC-MS (method 5): Rt=1.97 min; m/z=588 (M+H)+

### Example 372

### N-{3-[3-amino-2-(2,4-dichlorobenzoyl)-1-benzofuran-6-yl]benzyl}-N-(methylsulfonyl)glycin amide, hydrochloride

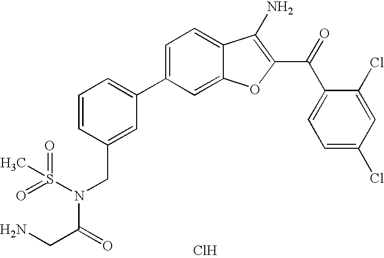

### Acylation:

100 mg (0.2 mmol) N-{3-[3-amino-2-(2,4-dichlorobenzoyl)-1-benzofuran-6-yl]benzyl}methane-sulfonamide is reacted with 411 mg (2 mmol) tert-butyl 2,5-dioxo-1,3-oxazolidine-3-carboxylate in the presence of 250 mg (2 mmol) DMAP as described in example 1. Instead of flash-chromatography preparative HPLC (method 1) is employed to purify the crude product.

Yield of the protected intermediate: 61 mg (46%)

### Cleavage of the Boc-Protecting Group to Give the Title Compound (Gly-Conjugate):

From 60 mg of the intermediate the Boc-protecting group is cleaved with a 13.5% solution of HCl in dioxane as described in example 1.

Yield of the title compound: 36 mg (67%)

TLC: dichloromethane/methanol/ammonia17% 15/1/0.1; Rf=0.22

HPLC (method 2): Rt=5.05 min;

LC-MS (method 4): Rt=1.86 min; m/z=546 (M+H)+

### Example 374

### N-{3-[3-amino-2-(2,4-dichlorobenzoyl)-1-benzofuran-6-yl]benzyl}-N-(methylsulfonyl)-L-valinamide, methanesulfonate

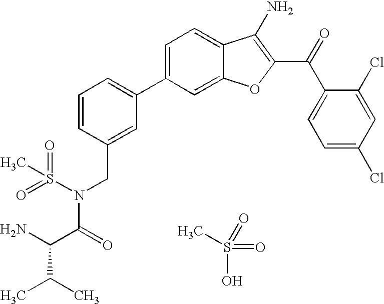

### Acylation:

N-{3-[3-amino-2-(2,4-dichlorobenzoyl)-1-benzofuran-6-yl]benzyl} methanesulfonamide is reacted with the respective Boc-protected amino acid N-carboxy anhydride in the presence of DMAP as described in example 1. The enantiomeric purity of the intermediate is sufficient (>95:5) and the protecting group is cleaved without further purification by chiral HPLC.

### Cleavage of the Boc-Protecting Group to Give the Title Compound (L-Valine-Conjugate):

From 150 mg of the intermediate (L-Val) the Boc-protecting group is cleaved with 105 mg (1.1 mmol) of methanesulfonic acid in 30 ml of dichloromethane. After 2 hours stirring at rt the solvent is removed and the title compound is precipitated from dichloromethane/dioxane with diethyl ether.

Yield of the title compound: 134 mg (90%)

TLC: dichloromethane/methanol/ammonia17% 15/1/0.1; Rf=0.41

HPLC (method 2): Rt=5.17 min;

LC-MS (method 5): Rt=1.94 min; m/z=588 (M+H)+

### Example 375

### N-{3-[3-amino-2-(2,4-dichlorobenzoyl)-1-benzofuran-6-yl]benzyl}-N-(methylsulfonyl)-L-lysinamide, dihydrobromide

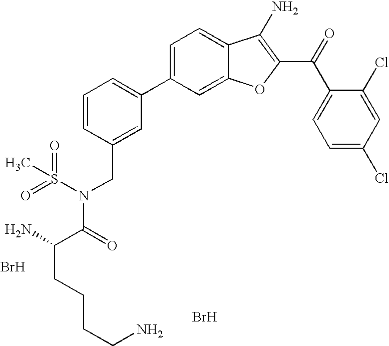

### Acylation:

100 mg (0.2 mmol) N-{3-[3-amino-2-(2,4-dichlorobenzoyl)-1-benzofuran-6-yl]benzyl}methane-sulfonamide is reacted with 830 mg (2 mmol) tert-butyl 4-(4-{[(benzyloxy)carbonyl]amino}butyl)-2,5-dioxo-1,3-oxazolidine-3-carboxylate in the presence of 250 mg (2 mmol) DMAP as described in example 1.

Yield of the protected intermediate: 152 mg (87%)

Separation of enantiomers by chiral HPLC; method A1, t1: 5.99 min, t2: 10.71 min

Enantiomeric ratio determined by chiral HPLC; method A1: 58:42 (L-Lys:D-Lys)

The enantiomers are separated by chiral HPLC (method A2).

### Cleavage of the Protecting Groups to Give the Title Compound (L-Lys-Conjugate):

The protecting groups are cleaved from 31 mg (0.036 mmol) of the separated L-configurated intermediate with 5 ml of a 33% solution of HBr in acetic acid. After 30 min stirring at RT the solvent is removed and the title compound is precipitated with diethyl ether.

Yield of the title compound: 29 mg (quant.)

TLC: acetonitrile/water/glacial acetic acid (5/1/0.2); Rf=0.1

HPLC (method 2): Rt=4.81 min;

LC-MS (method 4): Rt=1.72 min; m/z=617 (M+H)+

### Example 376

### N-{3-[3-amino-2-(2,4-dichlorobenzoyl)-1-benzofuran-6-yl]benzyl}-N-(methylsulfonyl)-D-lysinamide, dihydrobromide

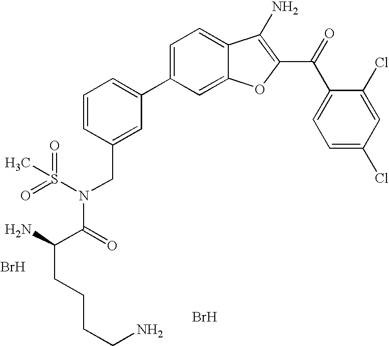

### Cleavage of the Protecting Groups to Give the Title Compound (D-Lys-Conjugate):

The protecting groups are cleaved from 26 mg (0.03 mmol) of the separated D-configurated intermediate in example 9 with 5 ml of a 33% solution of HBr in acetic acid. After 30 min stirring at RT the solvent is removed and the title compound is precipitated. It is purified by preparative HPLC (method 1). Respective fractions are collected, the solvent is removed in vacuo and the title compound is precipitated

Yield of the title compound: 6.4 mg (27%)

TLC: acetonitrile/water/glacial acetic acid (5/1/0.2); Rf=0.1

HPLC (method 2): Rt=4.8 min;

LC-MS (method 4): Rt=1.7 min; m/z=617 (M+H)+

### Example 377

### N-{3-[3-amino-2-(2,4-dichlorobenzoyl)-1-benzofuran-6-yl]benzyl}-N-(methylsulfonyl)-D-leucinamide, hydrochloride

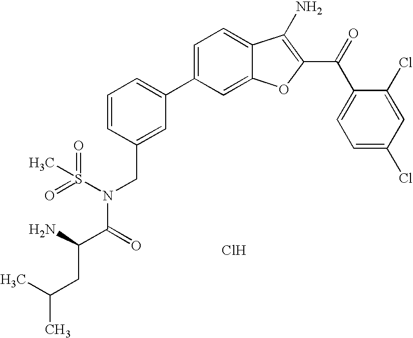

### Acylation:

400 mg (0.82 mmol) N-{3-[3-amino-2-(2,4-dichlorobenzoyl)-1-benzofuran-6-yl]benzyl}methane-sulfonamide is reacted with 2.1 g (8.2 mmol) tert-butyl 4-isobutyl-2,5-dioxo-1,3-oxazolidine-3-carboxylate in the presence of 1 g (2 mmol) DMAP as described in example 1.

Yield of the protected intermediate: 553 mg (96%)

Separation of enantiomers by chiral HPLC; method B1, t1: 3.61 min, t2: 4.44 min

Enantiomeric ratio: 71:29 (D-Leu:L-Leu)

The enantiomers are separated by chiral HPLC in preparative scale (method B2).

### Cleavage of the Boc-Protecting Group to Give the Title Compound (D-Leu-Conjugate):

The Boc-protecting group is cleaved from the separated Boc-protected intermediate (D-Leu enantiomer) with a 13.5% solution of HCl in dioxane as described in example 1.

Yield of the title compound: 149 mg

HPLC (method 2): Rt=5.29 min;

LC-MS (method 4): Rt=2.06 min; m/z=602 (M+H)+

### Example 378

### N-{3-[3-amino-2-(2,4-dichlorobenzoyl)-1-benzofuran-6-yl]benzyl}-N-(methylsulfonyl)-L-leucinamide, hydrochloride

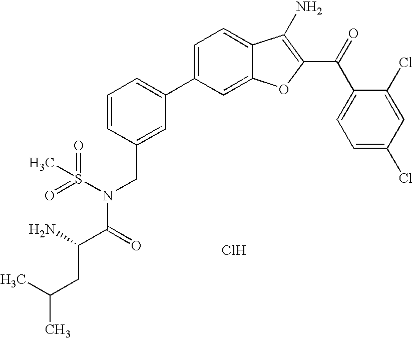

### Cleavage of the Boc-Protecting Groups to Give the Title Compound (L-Leu-Conjugate):

The Boc-protecting group is cleaved from the separated Boc-protected intermediate in example 11 (L-Leu enantiomer) with a 13.5% solution of HCl in dioxane as described in example 1.

Yield of the title compound: 38 mg.

HPLC (method 2): Rt=5.21 min;

LC-MS (method 5): Rt=2.04 min; m/z=602 (M+H)+

### Example 379

### N-{3-[3-amino-2-(2,4-dichlorobenzoyl)-1-benzofuran-6-yl]benzyl}-N-(methylsulfonyl)-L-alpha-asparagine hydrobromide

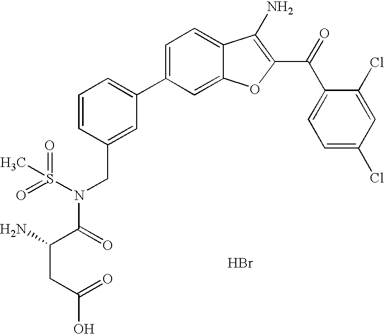

### Acylation:

100 mg (0.2 mmol) N-{3-[3-amino-2-(2,4-dichlorobenzoyl)-1-benzofuran-6-yl]benzyl} methane-sulfonamide is reacted with 714 mg (2 mmol) tert-butyl 4-[2-(benzyloxy)-2-oxoethyl]-2,5-dioxo-1,3-oxazolidine-3-carboxylate in the presence of 250 mg (2 mmol) DMAP as described in example 1. Instead of flash-chromatography preparative HPLC (method 1) is employed to purify the crude product.

Yield of the protected intermediate as an epimeric mixture: 73 mg (45%)

Separation of enantiomers by chiral HPLC; method A1, t1: 3.94 min, t2: 7.12 min

Enantiomeric ratio determined by chiral HPLC; method A1: 53:47 (L-Asp:D-Asp)

The enantiomers are separated by chiral HPLC (method A2).

### Cleavage of the Protecting Groups to Give the Title Compound (L-Asp-Conjugate):

The protecting groups are cleaved from 11.5 mg (0.014 mmol) of the separated L-configurated intermediate with 3 ml of a 33% solution of HBr in acetic acid. After 30 min stirring at RT the solvent is removed and the residue is lyophilised from dioxane/water.

Yield of the title compound: 9 mg (91%)

HPLC (method 2): Rt=4.97 min;

LC-MS (method 4): Rt=2.04 min; m/z=604 (M+H)+

### Example 380

### N-{3-[3-amino-2-(2,4-dichlorobenzoyl)-1-benzofuran-6-yl]benzyl}-N-(methylsulfonyl)-D-alpha-asparagine

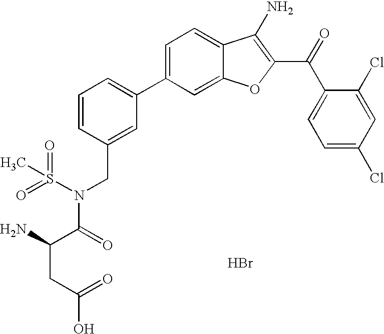

### Cleavage of the Protecting Groups to Give the Title Compound (D-Asp-Conjugate):

The protecting groups are cleaved from 12.4 mg (0.016 mmol) of the separated D-configurated intermediate in example 13 with 10 ml of a 33% solution of HBr in acetic acid. After 30 min stirring at RT the solvent is removed and the residue is lyophilised from dioxane.

Yield of the title compound: 11 mg (quant.)

HPLC (method 2): Rt=4.99 min;

LC-MS (method 4): Rt=2.04 min; m/z=604 (M+H)+

### Example 381

### N-{3-[3-amino-2-(2,4-dichlorobenzoyl)-1-benzofuran-6-yl]benzyl}-N-(methylsulfonyl)-L-valinamide, trifluoroacetate

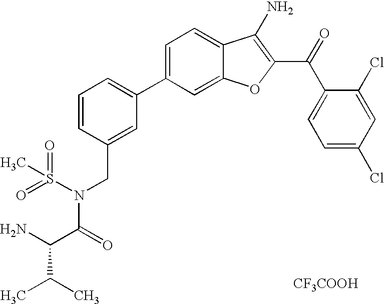

### Cleavage of the Protecting Group to Give the Title Compound (L-Val-Conjugate):

The Boc-protecting group is cleaved from 2.5 mg (0.0036 mmol) of the protected intermediate in example 1 with 1 ml of trifluoroacetic acid in 3 ml dichloromethane. After 30 min stirring at RT the solvent is removed and the residue is redistilled with acetonitrile.

Yield of the title compound: 2.5 mg (98%)

HPLC (method 2): Rt=5.16 min;

LC-MS (method 3): Rt=5.75 min; m/z=588 (M+H)+

### Example 382

### N-{3-[3-amino-2-(2,4-dichlorobenzoyl)-1-benzofuran-6-yl]benzyl}-N-(methylsulfonyl)glycin amide, trifluoroacetate

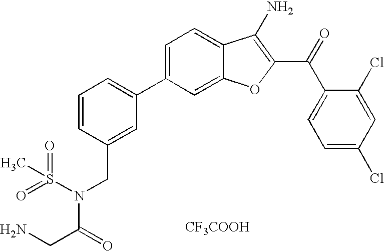

### Cleavage of the Protecting Group to Give the Title Compound (Gly-Conjugate):

The Boc-protecting group is cleaved from 10.1 mg (0.0156 mmol) of the protected intermediate in example 7 with 2 ml of trifluoroacetic acid in 2 ml dichloromethane. After 30 min stirring at RT the solvent is removed and the residue is lyophilised from dioxane.

Yield of the title compound: 9.5 mg (92%)

HPLC (method 2): Rt=5.05 min;

LC-MS (method 3): Rt=5.70 min; m/z=546 (M+H)+

### Proliferation-Assay

SK-N-DZ (human neuroblastoma; ATCC# CRL-2149) or U118 MG (human glioblastoma/astrocytoma; ATCC# HTB-15) or U87 MG (human glioma; ATCC# HTB-14) or H4 (human neuroglioma; ATCC# HTB 178) cells are cultivated under standard conditions (RPMI 1640/Glutamax 2 mM/glucose 2 g/l/SoBicarbonate 2 g/1/10% FCS; 37° C. in 5% CO₂ in a humid incubator). Cells are suspended using Accutase (PAA) and seeded at 1000 cells/Well in 100 μL medium/well in a 96-well plate (Greiner Kat. Nr. 655073). The test compound is added 24 Stunden after seeding. Commercial assays (CellTiter-Glo® Luminescent Cell Viability Assay; Promega Kat. Nr. G7571) are used 72 later according to the manufacturers instructions. IC₅₀ is calculated using GraphPad Prism.

Compounds are solved in 100% DMSO and diluted 1:20 in medium before used diluted 1:10 in the test.

The growth of neurological tumor cells is inhibited effectively.

| cell | IC50 (example 153) [M/L] |
|---|---|
| SK-N-DZ | 1.1e−7 (n = 4) |
| U118 MG | 1.2e−7 (n = 4) |
| U87 | 3.2e−7 (n = 2) |
| H4 | 1.7e−7 (n = 1) |

### Determination of Solubility and Stability

### Solubility:

The test compound is dissolved in an aqueous solution of 5% dextrose adjusted to the respective pH with 1n HCl. After shaking at room temperature for 24 h the probe is subjected to an ultra centrifugation process at 224000 g for 30 min. DMSO is added to the supernatant and the content of test compound is determined and quantified by HPLC employing a two point calibration curve of the test compound in DMSO.

HPLC Method: Agilent 1100 with DAD (G1315A), quat. Pump (G1311A), Autosampler CTC HTS PAL, Degaser (G1322A) and column thermostate (G1316A); Column: Zorbax Extend-C18 3.5μ; temperature: 40° C.; Eluent A: Water+5 ml Perchloric acid/1; Eluent B: Acetonitrile; flow rate: 0.7 mL/min; Gradient: 0-0.5 min 98% A, 2% B; Rampe: 0.5-4.5 min 10%, A 90% B; 4.5-6 min 10% A, 90% B; Rampe: 6.5-6.7 min 98% A, 2% B; 6.7-7.5 min 98% A, 2% B.

### Results:

For the compound of example 366 no degradation has been observed and the solubility has been determined:

| Vehicle | pH | SOL [mg/l] |
|---|---|---|
| 5% Dextrose | 3 | 360 |
| 5% Dextrose | 4 | 350 |

The solubility of the parent compound N-{3-[3-amino-2-(2,4-dichlorobenzoyl)-1-benzofuran-6-yl]benzyl}methanesulfonamide is below 0.1 mg/l.

### Stability:

Stability in Buffer at Different pH-Values

To determine the stability of test compounds at different pH-values 0.3 mg of the dry compound is dissolved in 0.5 ml of acetonitrile and the sample is sonified for 10 seconds. Then buffer solutions are added and the samples are sonified again.

### Chemical Composition of the Buffers:

pH 2: 0.03 M citric acid/0.0082 M hydrochloric acid/0.061 M sodium chloride
pH 3: 0.04 M citric acid/0.021 M sodium hydroxide/0.06 M sodium chloride
pH 4: 0.056 M citric acid/0.068 M sodium hydroxide/0.044 M Sodium chloride
pH 5: 0.096 M citric acid/0.2 M sodium hydroxide
pH 7: 0.029 M sodium hydroxide solution/0.05 M potassium dihydrogen phosphate
pH 10: 0.013 M sodium tetraborate/0.018 M sodium hydroxide

The test compound concentrations in solution is analyzed by HPLC every hour during 24 hours at 37° C.

### HPLC Method:

Agilent 1100 with DAD (G1314A), binary pump (G1312A), autosampler (G1329A), column thermostat (G1316A), thermostat (G1330A), Column: Kromasil 100 C18/60×2.1 mm/3.5 μm, Column temperature: 37° C., Eluent A: Water+5 ml perchloric acid/1, Eluent B: Acetonitrile, Gradient: 0-1.0 min 98% A, 2% B; 1.0-9.0 min 2% A, 98% B; 9.0-13.0 min 2% A, 98% B; 13.0-13.5 min 98% A, 2% B; 13.5-15.0 98% A, 2% B, Flow rate: 0.75 ml/min, Detection: 210 nm

### Results:

The results for the compounds of example 366 are shown below.

| | Buffer | Amount of remaining |
|---|---|---|
| Time | pH | compound of example 366 [%] |
| after 24 h at 37° C. | 2 | 100 |
| after 24 h at 37° C. | 3 | 100 |
| after 24 h at 37° C. | 4 | 94 |
| after 24 h at 37° C. | 5 | 58 |
| after 24 h at 37° C. | 7 | 4 |
| after 24 h at 37° C. | 10 | 2 |

### Stability in Rat and Human Plasma:

### Method:

Sample preparation included protein precipitation and stabilization of the example 366 to avoid decomposition of the compound during storage in the autosampler. In case of example 366 the precipitant solution contained 80% acetonitrile, 20% ammonium acetate buffer, pH 3, 2 mM AAC pH3 2 mM (v/v). 20 mL H3PO4 85% per liter precipitant solution were added to increase buffer capacity.

Sample preparation: For each sample calibration, quality control and unknown sample 600 μL precipitant solution was pipetted into a vial aliquots of 100 μL of whole blood was added directly after sampling, thoroughly mixed and subsequently centrifuged. An aliquot of the supernatant 100 μL was mixed with 100 μL ammonium acetate buffer, pH 3, 2 mM and was injected onto the HPLC column.

### HPLC Method:

Agilent 1100 with DAD (G1314A), binary pump (G1312A), autosampler (G1329A), column thermostat (G1316A), thermostat (G1330A), Column: RP-Select B+precolumn/125×4.6 mm/5 μm, Column temperature: 40° C., Eluent A: Water+0.25 ml H3PO4/l, Eluent B: Acetonitrile, Gradient: 0-5.0 min 60% B; 5.1-7.0 min 85% B; 7.1-10.0 min 60% B, Flow rate: 1.0 mL/min, Detection: 254 nm, Injection volume: 50 μL

### Results:

Compounds from example 366 is cleaved to more than 90% in rat and human plasma within 1 hour.

Pharmacokinetics (PK) in rat

Method: (as described above under “Stability in rat and human plasma”)

### Results:

Key PK parameter of released parent compounds after intravenous administration of prodrugs from example 366 (administration of 3 mg/kg test compound in saline at pH4).

| | |
|---|---|
| AUC [μg * h/mL] | 2.4 |
| T½ [h] | 4.2 |
| Vss [L/kg] | 7.3 |
| CL [L/h * kg] | 1.3 |

### Crossing Blood Brain Barrier:

Male Ncr nude mice (n=3/time point) is treated for 14 days orally with a therapeutically relevant dose of example 153 (200 mg/kg) or a vehicle control (time: Oh). Daily treatment with a high dose like this is necessary to effectively treat subcutaneously transplanted tumors in these mice. After treatment, mice are sacrified at indicated time points, blood and brain tissue is removed and analysed. The method is as described above in example 2 under “Stability in rat and human plasma”. It is demonstrated that the compound effectively crosses the blood brain barrier in these mice. Mean brain concentrations are indicated as absolute concentration and relative to plasma concentration at similar time points (from the same experiment) in the table below.

| | mean concentration | % of Plasma |
|---|---|---|
| Time [h] | [μg/mL] | concentration |
| 0 h | <0.1 | n.d. |
| 0.167 h | 0.6 | 9-19% |
| 0.5 h | 1.4 | 17-18% |
| 1.5 h | 1.0 | 12-90% |

Results: Example 153, if released into the plasma, crosses the blood brain barrier.

1. A method for treating or preventing cancers of the central nervous system, comprising administering a therapeutically effective amount of a compound of formula I to a subject in need thereof,
wherein said compound of formula I is 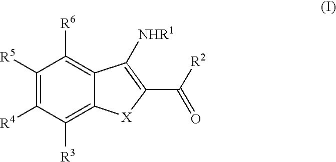
or a pharmaceutically acceptable salt, prodrug, or ester thereof,
wherein
X is selected from O and S;
R¹ is selected from H, (C₁-C₆)alkyl, C(O)(C₁-C₆)alkyl, and benzoyl;
R² is selected from
phenyl and naphthyl, each optionally substituted with 1, 2, or 3 substituents each independently selected from OH, CN, NO₂, (C₁-C₆)alkyl, (C₁-C₆)alkoxy, halo, halo(C₁-C₆)alkyl, halo(C₁-C₆)alkoxy, C(O)R^{A}, C(O)NR^{B}R^{B}, NR^{B}R^{B}, NH[(C₁-C₆)alkyl,]₀₋₁S(O)₂R^{B}, NH[(C₁-C₆)alkyl]₀₋₁C(O)R^{A}, and NH[(C₁-C₆)alkyl]₀₋₁C(O)OR^{B},
a heterocycle selected from a six membered heterocycle, a five membered heterocycle and a fused bicyclic heterocycle, each heterocycle being optionally substituted with 1, 2 or 3 substituents each independently selected from OH, CN, NO₂, (C₁-C₆)alkyl, (C₁-C₆)alkoxy, halo, halo(C₁-C₆)alkyl, halo(C₁-C₆)alkoxy, C(O)R A, C(O)NR^{B}R^{B}, NR^{B}R^{B}, NH[(C₁-C₆)alkyl]₀₋₁S(O)₂R^{B}, NH[(C₁-C₆)alkyl]₀₋₁C(O)R^{A}, and NH [(C₁-C₆)alkyl]₀₋₁C(O)OR^{B},
R^{A} is in each instance independently H, (C₁-C₆)alkyl, (C₁-C₆)alkoxy, NR^{B}R^{B}, or (C₁-C₆)alkyl, said alkyl being optionally substituted with OH, C(O)R^{B}, halo, (C₁-C₃)alkoxy, and NR^{B}R^{B};
R^{B} is in each instance independently H, (C₃-C₆)cycloalkyl, and (C₁-C₆)alkyl, said alkyl being optionally substituted with OH, ═O, halo, (C₁-C₆)alkoxy, NH(C₁-C₃)alkyl, N[(C₁-C₃)alkyl]₂, and NC(O)(C₁-C₃)alkyl,
and where R^{B}, when it is attached to a N atom, is in each instance (C₁-C₄)alkyl, then the 2 (C₁-C₄)alkyl groups, taken together with the N atom to which they are attached, may be joined together to form a saturated ring,
and where R^{B} and R^{B} together with the N to which they are attached may form a morpholinyl ring or a piperazinyl ring optionally substituted on the available N atom with (C₁-C₆)alkyl, said alkyl being optionally substituted with OH, ═O, NH₂, (C₁-C₆)alkoxy, NH(C₁-C₃)alkyl, or N[(C₁-C₃)alkyl]₂,
and with the proviso that when R^{B} is attached to S(O) or to S(O)₂, it cannot be H;
R³ is selected from H, OH, CN, (C₁-C₃)alkyl, (C₁-C₃)alkoxy, halo, halo(C₁-C₃)alkyl, and halo(C₁-C₃)alkoxy;
R⁴ is selected from
piperonyl,
Y where
Y is a heterocycle optionally substituted with 1, 2, or 3 substituents each independently selected from ═O, N-oxide, H, CN, NO₂, halo, halo(C₁-C₆)alkyl, OH, halo(C₁-C₆)alkoxy, C(O)OR^{B}, C(NH)NR^{B}R^{B}, NR^{B}R^{B}, S(O)₀₋₂R^{B}, S(O)₂NR^{B}R^{B}, (C₁-C₆)alkoxy, NR^{C}R^{C}, NR^{B}R^{E}, (C₁-C₆)alkyl, C(O)R^{D}
where said alkoxy being optionally substituted with 1 or 2 substituents selected from OH, NR^{B}R^{B}, and (C₁-C₃)alkoxy,
said alkyl being optionally substituted with CN, OH, ═O, halo, (C₁-C₆)alkoxy, C(O)R^{A}, NR^{B}R^{B}, NR^{C}R^{C}, NR^{B}R^{E}, C(NH)NR^{B}R^{B}, S(O)₀₋₂R^{B}, S(O)₂NR^{B}R^{B}, C(O)R^{B} C(O)OR^{B}, Z, C(O)Z, and C(O)N[(C₁-C₃)alkyl]Z, where Z in each instance is independently optionally substituted as described above,
R^{C} is selected from R^{B}, C(O)R^{B}, and S(O)₂R^{B},
R^{D} is selected from R^{A}, (C₃-C₆)cycloalkyl, Z and N[(C₁-C₃)alkyl]Z where
Z is in each instance a heterocycle independently optionally substituted with CN, ═O, OH, N-oxide, NO₂, halo, (C₁-C₆)alkoxy, halo(C₁-C₃)alkoxy, halo(C₁-C₃)alkyl, S(O)₂R^{B}, S(O)₂NR^{B}R^{B}, NR^{B}R^{B}, C(O)R^{A}, and (C₁-C₆)alkyl, said alkyl being optionally substituted with OH, C(O)R^{B}, (C₁-C₃)alkoxy and NR^{B}R^{B};
R^{E} is selected from C(O)R^{A}, C(O)R^{B}, S(O)₂R^{B}, S(O)₂NR^{B}R^{B} and C(O)[(C₁-C₆)alkyl]Z where Z is optionally substituted as described above,
phenyl and naphthyl each optionally substituted with 1, 2, or 3 substituents each independently selected from OH, CN, NO₂, halo, halo(C₁-C₆)alkyl, halo(C₁-C₆)alkoxy, C(O)OR^{B}, NR^{C}R^{C}, NR^{B}R^{E}, C(O)R^{D}, (C₁-C₆)alkoxy, C(NH)NR^{B}R^{B}, NR^{B}R^{B}, S(O)₀₋₂R^{B}, S(O)₂NR^{B}R^{B}, (C₁-C₆)alkyl, Z, C(O)Z
where Z is in each instance optionally substituted as described above,
said alkoxy being optionally substituted with 1 or 2 substituents selected from OH, NR^{B}R^{B}, and (C₁-C₃)alkoxy,
R^{C} is selected from R^{B}, C(O)R^{B}, and S(O)₂R^{B},
R^{D} is selected from R^{A}, (C₃-C₆)cycloalkyl, and N[(C₁-C₃)alkyl]Z where Z is optionally substituted as described above,
R^{E} is selected from C(O)R^{A}, C(O)R^{B}, S(O)₂R^{B}, S(O)₂NR^{B}R^{B} and C(O)[(C₁-C₆)alkyl]Z where Z is optionally substituted as described above,
said alkyl being optionally substituted with CN, OH, ═O, halo, (C₁-C₆)alkoxy, C(O)R^{A}, NR^{B}R^{B}, NR^{B}R^{E}, C(NH)NR^{B}R^{B}, S(O)₀₋₂R^{B}, S(O)₂NR^{B}R^{B}, C(O)R^{B}C(O)OR^{B}, Z, C(O)Z, and C(O)N[(C₁-C₃)alkyl]Z, where Z in each instance is independently optionally substituted as described above;
R⁵ and R⁶ are each independently selected from H, OH, CN, (C₁-C₃)alkyl, (C₁-C₃)alkoxy, halo, halo(C₁-C₃)alkyl, and halo(C₁-C₃)alkoxy.

2. The method of claim 1 wherein the compound of formula (I) is N-{3-[3-amino-2-(2,4-dichlorobenzoyl)-1-benzofuran-6-yl]benzyl}methanesulfonamide or a pharmaceutically acceptable salt, prodrug or ester thereof.

3. The method of claim 1 wherein the cancer of the central nervous system is selected from the group consisting of astrocytomas (differentiated and anaplastic), gliomas, gangliomas, pilocytic astrocytomas, oligodendrogliomas (differentiated and anaplastic), ependymomas, glioblastomas, multiforme, mixed gliomas, oligoastrocytomas, medulloblastomas, retinoblastomas, neurinomas (neurilemmoma), neurofibromas (Schwannoma), neuroblastomas, pituitary adenomas, meningiomas, heamangioblastomas, tumors of the plexus chorioideus, and brain metastases of other tumors.

4. A compound of the formula (IX) 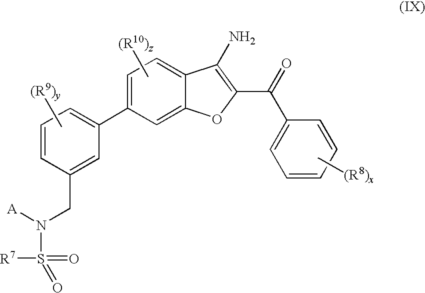
wherein
A is an alpha amino acid residue, which is linked via the α-carboxyl function, and which can optionally carry one or more protective groups,
R⁷ is H or (C₁-C₆)alkyl,
R⁸, R⁹ and R¹⁰ are independently selected from CN, NO₂, (C₁-C₆)alkyl, (C₁-C₆)alkoxy, halo, halo(C₁-C₆)alkyl and halo(C₁-C₆)alkoxy, and
x, y, z are independently 1, 2, or 3,
or a pharmaceutical acceptable salt thereof.

5. The compound of claim 4 wherein
A is a naturally occurring alpha amino acid residue of the D or L configuration, which is linked via the α-carboxyl function, and which can optionally carry one or more protective groups.

6. The compound of claim 4 wherein
A is a naturally occurring alpha amino acid residue of the D or L configuration selected from the amino acids glycine, alanine, leucine, valine, norleucine, isoleucine, D-allo isoleucine, lysine, histidine, ornithine, arginine, aspartic acid, asparagine, glutamic acid, glutamine, serine, threonine, phenylalanine, and tyrosine, which is linked via the α-carboxyl function, and which can optionally carry one or more protective groups.

7. The compound of claim 4 which is selected from:
N-{3-[3-amino-2-(2,4-dichlorobenzoyl)-1-benzofuran-6-yl]benzyl}-N-(methylsulfonyl)-L-valinamide,
N-{3-[3-amino-2-(2,4-dichlorobenzoyl)-1-benzofuran-6-yl]benzyl}-N-(methylsulfonyl)-L-isoleucinamide,
N-{3-[3-amino-2-(2,4-dichlorobenzoyl)-1-benzofuran-6-yl]benzyl}-N-(methylsulfonyl)-D-alloisoleucinamide,
N-{3-[3-amino-2-(2,4-dichlorobenzoyl)-1-benzofuran-6-yl]benzyl}-O-benzyl-N-(methylsulfonyl)-L-serinamide,
Benzyl N-{3-[3-amino-2-(2,4-dichlorobenzoyl)-1-benzofuran-6-yl]benzyl}-N-(methyl sulfon-yl)-L-alpha-asparaginate,
N-{3-[3-amino-2-(2,4-dichlorobenzoyl)-1-benzofuran-6-yl]benzyl}-N-(methylsulfonyl)-D-valinamide,
N-{3-[3-amino-2-(2,4-dichlorobenzoyl)-1-benzofuran-6-yl]benzyl}-N-(methylsulfonyl)glycin amide,
N-{3-[3-amino-2-(2,4-dichlorobenzoyl)-1-benzofuran-6-yl]benzyl}-N-(methylsulfonyl)-L-lysinamide,
N-{3-[3-amino-2-(2,4-dichlorobenzoyl)-1-benzofuran-6-yl]benzyl}-N-(methylsulfonyl)-D-lysinamide,
N-{3-[3-amino-2-(2,4-dichlorobenzoyl)-1-benzofuran-6-yl]benzyl}-N-(methylsulfonyl)-D-leucinamide,
N-{3-[3-amino-2-(2,4-dichlorobenzoyl)-1-benzofuran-6-yl]benzyl}-N-(methylsulfonyl)-L-leucinamide,
N-{3-[3-amino-2-(2,4-dichlorobenzoyl)-1-benzofuran-6-yl]benzyl}-N-(methylsulfonyl)-L-alpha-asparagine,
N-{3-[3-amino-2-(2,4-dichlorobenzoyl)-1-benzofuran-6-yl]benzyl}-N-(methylsulfonyl)-D-alpha-asparagine,
or a pharmaceutical acceptable salt thereof.

8. Process for preparing a compound of formula (IX) as defined in claim 4 by a reaction between a compound of formula (X) 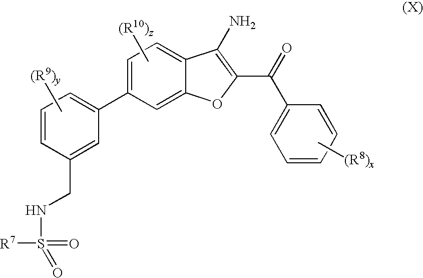
and a compound of formula (XI) 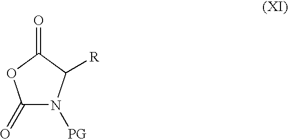
followed by a deprotection of the protective group PG,
wherein
R is defined by the side chain of the alpha amino acid which might carry further protective groups as defined above in the compound of the formula (IX),
PG is a protective group selected from tert-butoxycarbonyl (Boc) and benzyloxycarbonyl (Z).

9. A method for treating or preventing cancers of the central nervous system, comprising administering a therapeutically effective amount of a compound of claim 4 to a subject in need thereof.

10. The method of claim 9 wherein the cancer of the central nervous system is selected from the group consisting of astrocytomas (differentiated and anaplastic), gliomas, gangliomas, pilocytic astrocytomas, oligodendrogliomas (differentiated and anaplastic), ependymomas, glioblastomas, multiforme, mixed gliomas, oligoastrocytomas, medulloblastomas, retinoblastomas, neurinomas (neurilemmoma), neurofibromas (Schwannoma), neuroblastomas, pituitary adenomas, meningiomas, heamangioblastomas, tumors of the plexus chorioideus and brain metastases of other tumors.

11. Combination comprising at least one compound of claim 4 and at least one anti-hyper-proliferative agent, anti-epileptic agent, agent acting against brain edemas, analgesic, antidepressant and/or immunomodulating agent.

12. A method for treating, preventing or managing of cancers of the central nervous system, comprising administering a therapeutically effective amount of the combination of claim 11 to a subject in need thereof.

13. Pharmaceutical composition comprising the combination of claim 11.

14. (canceled)

15. Pharmaceutical composition comprising the compound of claim 4.
